(19) United States
(12) Patent Application Publication
(10) Pub. No.: US 2008/0171728 A1
(43) Pub. Date: Jul. 17, 2008

(54) Efficient Process for Preparing Steroids and Vitamin D Derivatives With the Unnatural Configuration at C20 (20 Alpha-Methyl) from Pregnenolone

(75) Inventor: Alexander James Bridges, Saline, MI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: QuatRx Pharmaceuticals Co.

(21) Appl. No.: 12/013,231

(22) Filed: Jan. 11, 2008

Related U.S. Application Data
(60) Provisional application No. 60/915,186, filed on May 1, 2007.
(60) Provisional application No. 60/884,661, filed on Jan. 12, 2007.

(51) Int. Cl. A61K 31/59; C07C 401/00

(52) U.S. Cl. 514/167; 552/653

(57) ABSTRACT

Disclosed herein are methods for preparing steroids and Vitamin D derivatives having the unnatural beta (usually S) configuration at C20, the methods comprising the use of compounds of the formula:

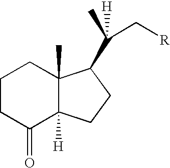

wherein R is as defined herein. Also disclosed are steroids and Vitamin D derivatives made using the methods disclosed herein and pharmaceutical compositions comprising said steroids and Vitamin D derivatives.

### FIELD OF THE INVENTION

Methods for preparing Steroids and Vitamin D derivatives with the unnatural beta (usually S) configuration at C20 from Pregnenolone are disclosed. The methods are used to synthesize (20S)-1α-hydroxy-2-methylene-19-norbishomopregnacalciferol and other related compounds. Several intermediates and pharmaceutical compositions comprising the steroids and Vitamin D derivates made using the methods disclosed herein are also described.

### BACKGROUND OF THE INVENTION

In recent years certain steroid derivatives, but especially Vitamin D derivatives, have been shown to have very interesting biological properties if the 21-methyl group in the C17-steroidal side chain is inverted from the natural α, usually 20R, configuration to the unnatural β, usually 20S, configuration. There are many published ways of introducing the unnatural 20S stereochemistry into steroids, but they all suffer from one (or more) of four problems. First, the starting material is expensive, or requires extensive chemical manipulation. Second, the synthetic procedure will be long, and require multiple chromatographies, thereby making the cost of goods produced through said synthetic scheme exorbitant. Third, the synthesis may contain steps or reagents that are not readily used on an industrial scale. And fourth, the synthesis may not provide the desired product in acceptable yields or stereochemical purity for use as a drug substance.

The Applicants disclose herein a chemical process for introducing the unnatural, usually S 20 methyl configuration (21-epi) into the C17 steroidal side chain of steroidal 5,7-dienes, which are the precursors of Vitamin D and its many analogues. This method allows for the elaboration of the steroidal side chain in good overall yield and stereochemical purity, and utilizes a cheap steroid starting material, pregn-5-en-3β-ol-20-one, which is 1) available in ton quantities, 2) one of the cheapest steroids commercially available, and as a result 3) is an excellent starting material for industrial processes. The method further uses intermediates that are solids, most of which can be purified to a high degree by recrystallization from commonly used industrial solvents, or by simple column chromatography.

Described herein are methods useful in converting pregn-5-en-3β-ol-20-one, and certain of its simple derivatives to a steroidal 5,7-diene with a partially or completely C20-homologated side chain with the unnatural β-configuration (usually S, 21-epi) of the C21 substituent (usually C21 methyl). This diene is then converted to the corresponding 21β-Vitamin D derivative, using a well established photochemical, and thermal process, which is used industrially on a very large scale to convert 7-dehydrocholesterol to Vitamin D₃ and ergosterol to ergocalciferol. For some Vitamin D derivatives, this will complete the synthesis, but for many, especially those with non-natural A-ring moieties, the unwanted A-ring can now be removed oxidatively in a well established process to produce a Windhaus-Grundmann ketone, with the overall photolysis-rearrangement-ozonolysis sequence leading to a scission of the A and B rings and the C8 position of the steroid being converted to a ketone. The desired A ring and seco-B ring can be added back using chemistry well established in the art, to make the desired, unnatural A-ring containing Vitamin D with the β-configuration at C21. Two sequences to make the desired steroidal diene are described, which differ in the order in which the double bond is introduced, and when the side chain construction is performed, are described herein. The processes are enabled by disclosing a full synthesis of (20S)-1α-hydroxy-2-methylene-19-norbishomopregnacalciferol, (Becocalcidiol). The use of this technology to make other known, and many novel Vitamin D and steroid derivatives is also revealed herein. Also described are some alternative ways of degrading C21-β steroids to Vitamin D precursors with retention of the C6 and C7 carbons.

### SUMMARY OF THE INVENTION

For the production of (20S)-1α-hydroxy-2-methylene-19-norbishomopregnacalciferol, the sequence, which introduces the 7,8-double bond before elaborating the C17 side chain, is more efficient, and more convenient than the sequence, whereby the 7,8-double bond is introduced after the C17 side chain has been elaborated. Either variant of this method can be used to prepare a large number of 20β-methyl (20-epi) Vitamin D derivatives, by simple extensions of the key processes described herein. For example, as described herein, an unmodified A ring Vitamin D precursor can be made and turned into the 3β-hydroxy Vitamin D analogue by simple photolysis and deprotection of the key C20 homologated pregna-5,7-diene derivatives described herein. Or in another manifestation, by using chemistry obvious to one skilled in the art, one can convert pregn-5-en-3β-ol-20-one, or other suitable 20-ketosteriod precursor into an appropriately diprotected 1α,3β-pregn-5-endiol-20-one derivative, which can then be 7,8-dehydrogenated using methods described herein, and then C20 homologated to the appropriate 20β-methyl (20-epi) steroidal 5,7-diene, which can be photolysed and deprotected to give the desired 1α,3β-20-epi Vitamin D analogue. Alternatively, the 3β,20β-Vitamin D derivative can be 1α-hydroxylated using an isomerization-allylic hydroxylation-reisomerization sequence. Another example of the utility of this method is to photolyse the steroidal 5,7-diene produced by this process to the Vitamin D triene, and ozonize it, and then do a Lythgoe or Julia coupling on the resultant CD-ring ring Windaus-Grundmann ketone, to produce a 20-epi Vitamin D analogue with a non-natural A-ring substitution pattern. This latter exemplification of the method also provides the desired bicycle (below) in improved chemical yield and acceptable stereochemical purity over the currently published methods. A minor variation on this sequence allows for the C17 21-epi side chain to be built onto the steroidal nucleus, and the AB-ring scission is then carried out by ozonolysis of the steroidal monoene, followed by a Norrish type II photochemical cleavage to give a norsteroid which still contains C6 and C7 of the B-ring. This can then be converted to a 21-epi Vitamin D derivative by methods described in the literature.

In a broad aspect methods of converting pregnenolone (1) into (1R,7αR)-1-sec-butyl-7a-methylhexahydro-1H-inden-4(2H)-one (where R is H) and which has the following structure

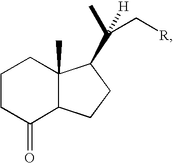

or into derivatives thereof, where
R is alkyl, alkenyl, alkynyl, —O-alkanoyl, alkoxy, alkoxyalkoxy, —O-silyl (where the silyl group includes such groups as TMS, TBDMS, TPS, TIPS, and TBDPS), OH, cycloalkyl, aryl, heteroaryl, or heterocycloalkyl, wherein each is optionally substituted with one or more groups that are independently alkyl, halogen, alkoxy, amino, monoalkylamino, dialkylamino, cyano, —O-trityl, —O-pivaloyl, or other alcohol protecting groups known in the art.

In another aspect, disclosed is the use of pregnenolone (1) to produce O-protected 20R,22-homopregnen-22-al (2) and O-protected 20R,22-homopregnen-22-ol (3) derivatives in good overall yield, and high diastereomeric purity at C20, where the protecting groups are preferably silyl ethers.

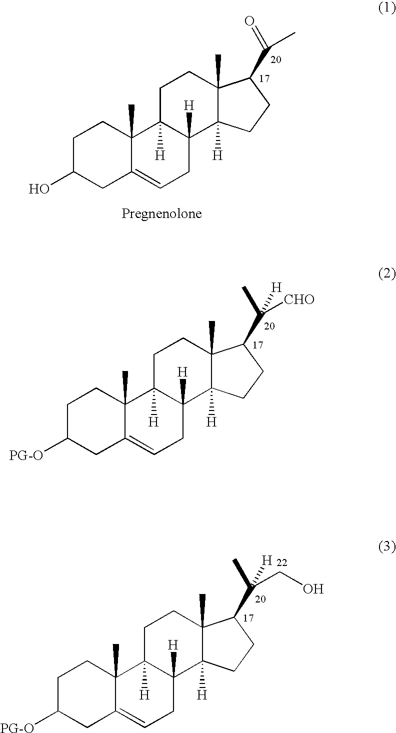

In another aspect, disclosed is the use of pregn-5-en-3β-ol-20-one (1) to produce 3,O-protected 20R,22-homopregna-5,7-dien-22-al (4) and 3,O-protected 20R,22-homopregna-5,7-dien-22-ol (5) derivatives in good overall yield, and high diastereomeric purity at C20.

In another aspect, disclosed is the use of pregn-5-en-3β-ol-20-one (1) to produce pregna-5,7-dien-3β-ol-20-one (6) in a high yielding, short and convenient, synthetic process.

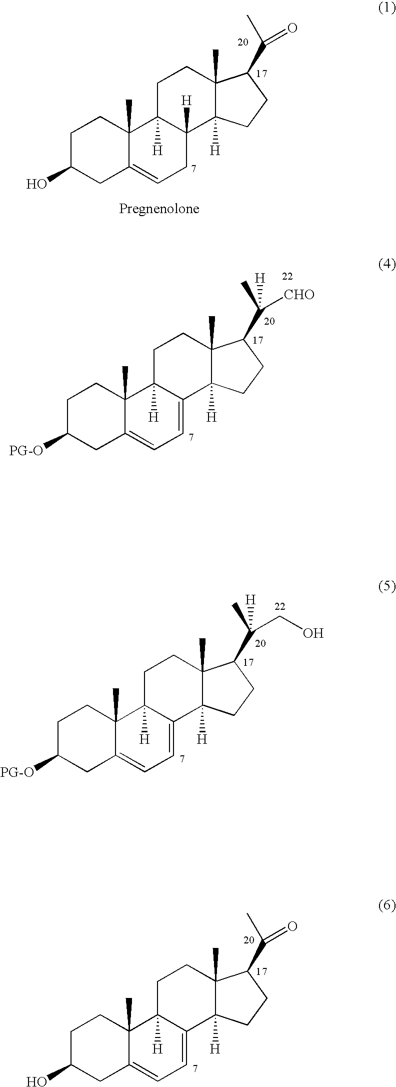

These compounds are useful in the production of unnatural C20 configuration, (usually S stereochemistry), steroid derivatives, especially Vitamin D derivatives. These Vitamin D derivatives can also be elaborated from the key intermediates, (2), (3), (4) and (5) described herein, all of which contain the desired chirality at C20, using a wide variety of methods, for example as described in “Synthesis of Vitamin D (Calciferol)” Zhu, G.-D., Okamura, W. H. Chem. Rev., 95 1877-1952, (1995). In turn, the convenient and efficient synthesis of (2-5) from pregn-5-en-3β-ol-20-one is also described herein. For example, the aldehyde (2) may be homologated into a very wide variety of steroidal side chains, for example by being reacted with a Grignard reagent, or an olefinating reagent, or a primary or secondary amine and a reducing agent, or an enolate, etc., or reduced to alcohol (3) with an appropriate reducing agent. In turn, the alcohol moiety in (3) may be reacted to form an ether, or an ester, or it may be converted into a leaving group, such as a sulfonate ester or a halide and then reacted with a nucleophile, which may be used to install a C22-C23 carbon, nitrogen, oxygen, phosphorus or sulfur bond. Furthermore C22 halides (see below) can be transformed into C22 metal species, which further adds to the synthetic utility of this invention, using many electrophilic agents, obvious to one skilled in the art. Consequently, the above method affords a practical and cost effective entry into a vast array of possible C20-epi steroidal and Vitamin D side chains, each having its own unique biological activity. This concept is illustrated by a synthesis of the C20-epi-C22,C23-bishomopregnacalciferol precursor (1R,3αR,7αR)-7-methyl-1-([1S]-methylprop-1-yl)octahydroinden-4-one, and its subsequent conversion by known chemistry to (20S)-1α-hydroxy-2-methylene-19-norbishomopregnacalciferol but is not limited in any way to this particular manifestation.

Pharmaceutical compositions comprising the steroids and/or Vitamin D analogues made using the methods of the invention or compounds disclosed herein are also contemplated.

### DETAILED DESCRIPTION

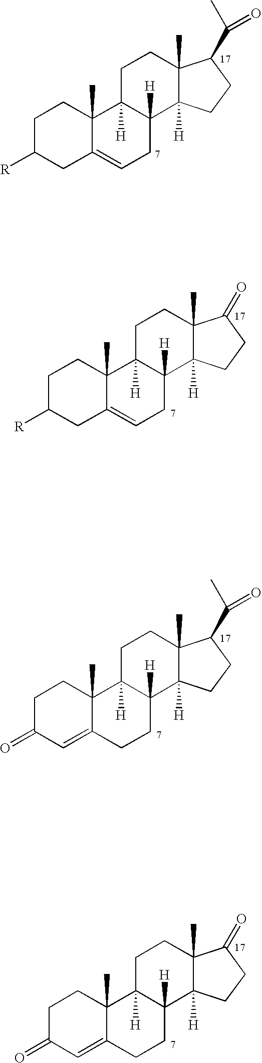

The conversion of the most suitable, commonly available and cheap steroids (typical examples of which are illustrated above) into precursors for Vitamin D requires two separate sets of chemical transformation of the steroid. These steroids do not have a large C17 side chain, as natural steroid-cleaving Cyp enzymes degrade most steroids to either a C17 ketone (eg androgens, estrogen, DHEA) or to a C17 acetyl group, (eg pregnenolone, progesterone, possibly hydroxylated as in the corticosteroids). Therefore, the desired C17 side chain has to be built up from the C17 keto or acetyl function. Herein we describe how to do that efficiently from the 17-acetyl group for compounds which have the unnatural β-(epi) methyl group at C20, although the methodology can be extended to include stereospecific syntheses of the natural C20 configuration, as discussed herein. The other functionality crucial for Vitamin D synthesis by the usual commercial processes is a B-ring 5,7-diene, and this functionality is missing from all commonly available steroids, except for certain plant steroids, which do not contain a 17-keto or acetyl group. Therefore, this functionality also has to be introduced. There are techniques described in the literature to introduce this diene system either from the 5-ene steroids, or from the 3-oxo-4-ene steroids. Herein we describe how to introduce a C17 side chain for 20-epi-steroids, and then subsequently, for specific Vitamin D analogue synthesis, introduce the 7,8-double bond, using the readily available and cheap 17-acetyl-5-ene steroid pregn-5-en-3β-ol-20-one, as illustrated in Scheme 1 below.

In a first aspect (Scheme 1), pregnenolone (also called pregn-5-en-3β-ol-20-one) (1)

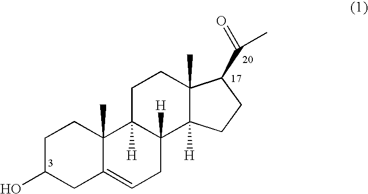

is used to prepare a compound of the formula:

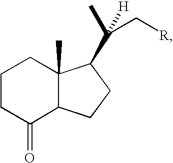

where R is as defined above, the method comprising
a) reacting the 3-hydroxy group of pregnenolone with a protecting group to form a compound of the formula:

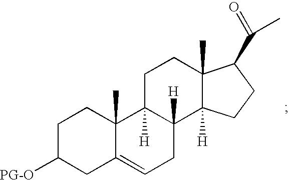

b) converting the product from step a) into a compound of the formula:

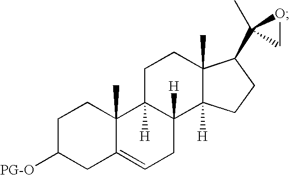

c) converting the product from step b) into a compound of the formula:

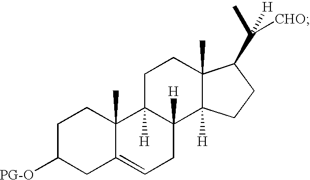

d) converting the product from step c) into a compound of the formula:

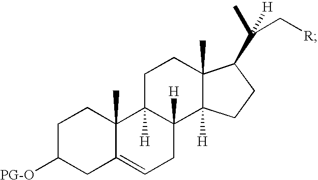

e) converting the product from step d) into a compound of the formula:

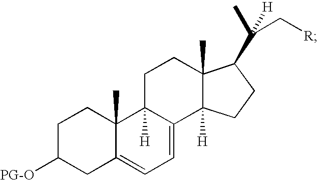

f) converting the product from step e) into a compound of the formula:

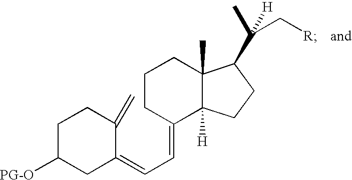

g) converting the product from step f) into the desired product.

In an embodiment of the first aspect, R is methyl.

In another embodiment of the first aspect, PG is a C₁-C₄ alkyl, benzyl or silyl group.

In still another embodiment of the first aspect, PG is a silyl group that is TBS, TES, or TIPS.

In an embodiment of the first aspect, when R is methyl, the product of step c) is converted to the product of step d) by treatment with CH₂═S(CH₃)₂, in a solvent, at low temperature.

In another embodiment of the first aspect, R is C₁-C₆ alkyl, C₂-C₆alkenyl, C₂-C₆ alkynyl, —O—C₂-C₆ alkanoyl, C₁-C₆ alkoxy, C₁-C₄ alkoxy C₁-C₄ alkoxy, —O-TBS, —O-TIPS, —O-TES, OH, C₃-C₆cycloalkyl, phenyl, pyridyl, thiazolyl, pyrimidyl, piperidinyl, pyrrolidinyl, morpholinyl, wherein each (except for H) is optionally substituted with one or more groups that are independently alkyl, halogen, alkoxy, OH, amino, monoalkylamino, dialkylamino or cyano.

In yet another embodiment of the first aspect, the 3-hydroxyl protecting group is a silyl group (such as TIPS, TES, TBS or TMS), benzyl, or C₁-C₄ alkoxy.

In another embodiment of the first aspect, R is methyl.

In another embodiment of the first aspect, R is suitably hydroxyl protected 3-hydroxy-3-methylbutyl, 3-hydroxy-3-ethylpentyl, 2-(1-hydroxycyclopenyl)ethyl, 4,4,4-trifluoro-3-hydroxy-3-(trifluoromethyl)butyl.

In yet another embodiment of the first aspect, PG is a silyl group.

In yet still another embodiment of the first aspect, PG is t-butyldimethylsilyl (abbreviated as TBS or TBDMS), triethylsilyl (abbreviated as TES) or triisopropylsilyl (abbreviated as TIPS) group and R is methyl.

In another embodiment of the first aspect, the epoxidation of the product from step a) is carried out by treating the methyl ketone with methyl sulfonium ylide in a solvent. Suitable solvents include THF. The ylide can be generated from dimethylsulfonium iodide or bromide and a strong base, such as KHMDS.

In another embodiment of the first aspect, the epoxidation of the product from step a) is carried out by treating the methyl ketone with methyl sulfonium ylide in a solvent at low temperatures in the range of about −40° C. to about −80° C. Suitable solvents include THF-toluene mixtures.

In yet another embodiment of the first aspect, the conversion of the epoxide from step b) to the aldehyde is performed using a Lewis acid, such as BF₃ etherate, BCl₃, MgCl₂, MgBr₂, Al(OPrⁱ)₃, Ti(OPrⁱ)₄, titanocene dichloride, ZnCl₂ etherate, GaCl₃, and In(OTf)₃ or Lewis acidic reagents (which cause the epoxide to rearrange to the aldehyde, and then react with the aldehyde in situ) such as MeMgBr, TMSCH₂MgCl, TMSCH₂MgBr, BH₃/BF₃, BH₃/BCl₃, Tebbe reagent, Petasis reagent, and DIBAL-H. A preferred Lewis acid is MgBr₂. Non-polar solvents, such as toluene are also preferred. Reaction temps between about −20° C. and 0° C. are also preferred. MgBr₂, in toluene at about −10° C. is also preferred.

In still another embodiment of the first aspect, the aldehyde is optionally reacted with an olefinating reagent (such as methylenetriphenylphosphorane, ethylidenetriphenylphosphine, trimethylsilylmethyllithium, carbon tetrabromide/triphenylphosphine, 1-lithiotrimethylphosphonoacetate, organometallic reagents such as the Grignard reagents, methylmagnesium bromide, methylmagnesium chloride, isopentyl magnesium bromide, phenylmagnesium iodide or bromide, vinylmagnesium bromide, and organolithium compounds such as methyl lithium, 2-thienyllithium, allyl lithium and phenyl lithium, a reducing agents, such as NaBH₄, Ca(BH₄)₂, NaCNBH₃ or LAH (in one embodiment, the epoxide rearrangement to form the aldehyde and the reduction of the aldehyde to an alcohol are performed in a one pot reaction, without isolation of the aldehyde); directed aldol reaction conditions, such as the use of preformed lithium, silyl or boron enolates, all well known to one skilled in the art. Additional specific examples of compounds, where PG or PG* is TBS, TIPS or acetate may be found below.

Furthermore, many Vitamin D derivatives, with the C19 methylene group, and possible 1α-hydroxyls, can be made directly from the steroidal monoene and diene and the Vitamin D triene intermediates claimed in the scheme above. Much chemistry has been described in the Vitamin D area to modify the A-ring of steroidal Vitamin D precursors exactly analogous to those claimed above, and all of this chemistry may be used with the current invention to produce 20-epi isomers of these known compounds. In such cases, examples of R include, but are not limited to, methyl, ethyl, 3-methylbutyl, 3-hydroxy-3-methylbutyl, 3-hydroxy-3-ethylpentyl, 2-(1-hydroxycyclopenyl)ethyl, 4,4,4-trifluoro-3-hydroxy-3-(trifluoromethyl)butyl, E,E,3-hydroxy-3-ethylpent-2-enyliden-1-yl, E-2R-2-cyclopropyl-2-hydroxyethyliden-1-yl, with hydroxyls suitably protected using chemistry known in the art.

In still another embodiment of the first aspect, the 7-position is brominated with a brominating reagent, such as 1,3-dibromo-5,5-dimethylhydantoin (“Bromantin”, “DMDBH”), or NBS. DMDBH is a preferred brominating agent. The 7-bromo compound may then be subjected to base-induced dehydrobromination conditions, thereby generating the diene. Alternatively, the 7-bromo compound is reacted with an aryl sulfide (such as, for example 4-chlorophenylthiol) thereby forming a 7-thioether with is oxidized to the sulfoxide using an oxidizing agents, such as MCPBA or oxone. The sulfoxide is then heated in the presence of a base, such as TEA, Hunig's base, or pyridine, thereby generating the 5,7-diene.

In yet still another embodiment of the first aspect, the diene produced above is photolyzed first at a short wavelength, then at a longer wavelength, and then the resulting triene is thermally equilibrated, as is known in the art. The Vitamin D triene so produced may be the desired product or a protected form thereof, or it may be ozonolyzed to form the desired Windhau-Grundmann ketone product.

All references disclosed herein are incorporated by reference.

We also describe a variation of this method using pregn-5-en-3β-ol-20-one in the synthesis of 20-epi-Vitamin D derivatives, which introduces the double bond before the C17 side chain is elaborated (see Scheme 2, below).

Alternatively, in a second aspect, pregnenolone (1) can be used to produce a compound of the formula (Scheme 2):

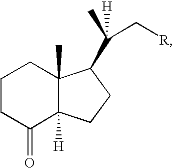

where R is as defined above, via a method comprising
a) reacting the 3-hydroxy group of pregnenolone with a protecting group to form a compound of the formula:

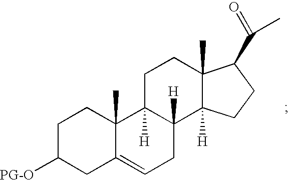

b) converting the product from step a) into a compound of the formula:

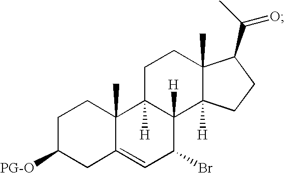

c) converting the product from step b) into a compound of the formula:

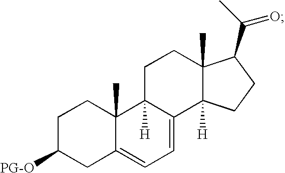

d) optionally (if necessary for removal or exchange of the protecting group, the need for which is understood by one of skill in the art) converting the product from step c) into a compound of the formula:

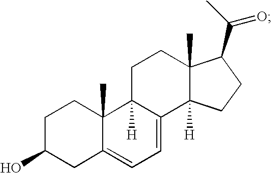

e) optionally (if necessary for exchange of the protecting group converting the product from step d) into a compound of the formula:

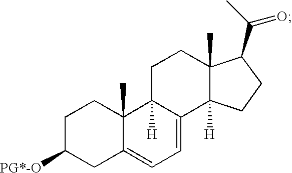

f) converting the product from step e) into a compound of the formula, where PG and PG* may be the same or different:

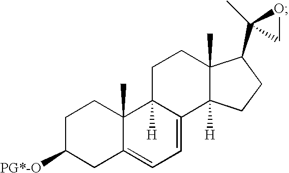

g) converting the product from step f) into a compound of the formula:

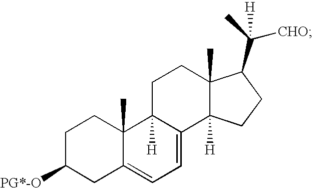

h) converting the product from step g) into a compound of the formula:

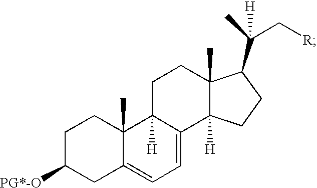

i) converting the product from step g) into a compound of the formula:

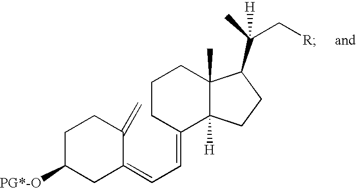

j) converting the product from step h) into the desired product.

In a further embodiment, the first and second aspects also entail reducing the ketone of the formula:

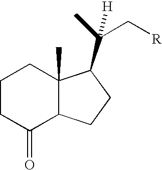

to an alcohol of the formula:

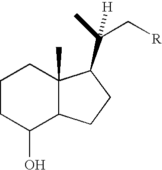

by treatment with a reducing agent. The reducing agent may be LAH, NaBH₄, Ca(BH₄)₂, or a transition metal catalyst and hydrogen.

In yet another embodiment of the second aspect, PG is a silyl group, C₁-C₄ alkyl (such as methyl), benzyl optionally substituted with one or two OCH₃ groups, or an alkanoyl protecting group and PG* is a silyl protecting group.

In still another embodiment of the second aspect, PG is acetate and PG* is the t-butyldimethylsilyl group.

In yet still another embodiment of the second aspect, PG is acetate and PG* is the t-butyldimethylsilyl group and R is methyl.

In another embodiment of the second aspect, when R is methyl, the epoxidation of the product from step e) is carried out by treating the methyl ketone with methyl sulfonium ylide (CH₂═S(CH₃)₂) in a solvent. Suitable solvents include THF. The ylide can be generated from dimethylsulfonium iodide or bromide and a strong base, such as KHMDS. The reaction is also performed at low temperature, such as about −80° C. to about −20° C., optionally in the presence of a cosolvent, such as toluene.

In still another embodiment of the second aspect, the solvent is THF and PG* is a TBDMS or TIPS group.

In yet another embodiment of the second aspect, PG is acetate and PG* is TIPS.

In still another embodiment of the second aspect, PG and PG* are both TBS or TIPS.

The synthetic sequences from the first and second aspects can be used to make the following compounds:

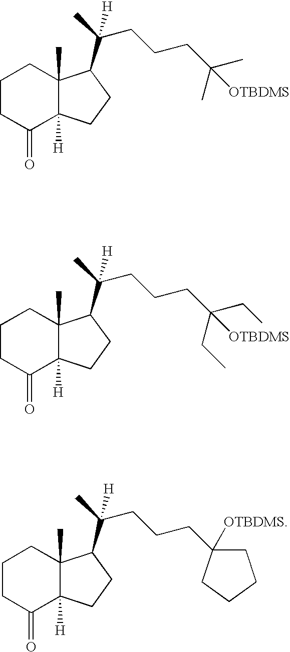

Both the sequences shown in Scheme 1 and Scheme 2 have been used to prepare 20S,3β-(trialkylsiloxy)-22,23-bishomopregna-5,7-dienes (15) and (39), the key steroidal diene intermediates for the synthesis of (20S)-1α-hydroxy-2-methylene-19-norbishomopregnacalciferol (52) (Becocalcidiol). In this synthesis it is advantageous to introduce the 7,8-double bond directly into pregnenolone rather than into the fully C17-elaborated steroid, as this order is more efficient overall, as well as operationally simpler to carry out, making Scheme 2 preferable to Scheme 1.

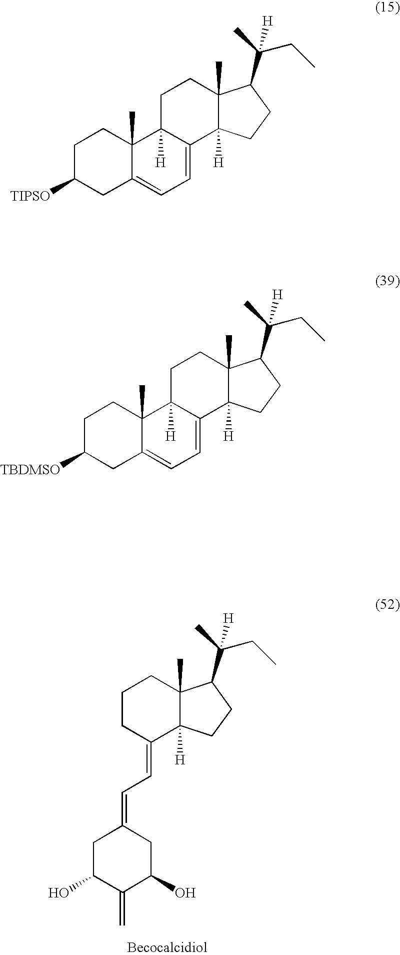

In another aspect, disclosed herein is a method of preparing 20S-1α-hydroxy-2-methylene-22,23-bishomopregnacalciferol comprising reacting

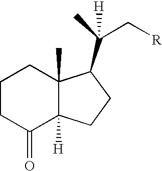

where R is methyl; with

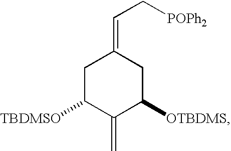

followed by a desilylation process

One of skill in the art will appreciate that silyl groups, such as TIPS could be used instead of TBDMS.

The methods of the first and second aspects may be used to make the compounds of the formulas:

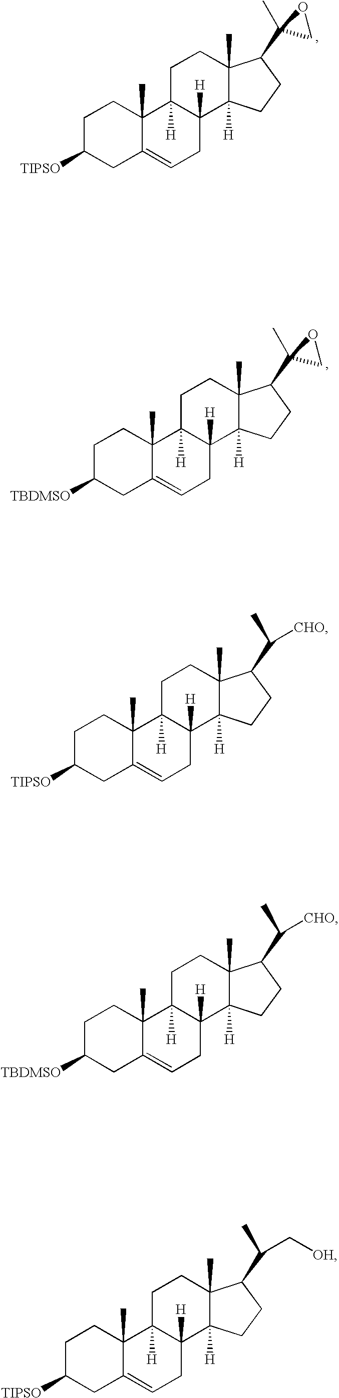

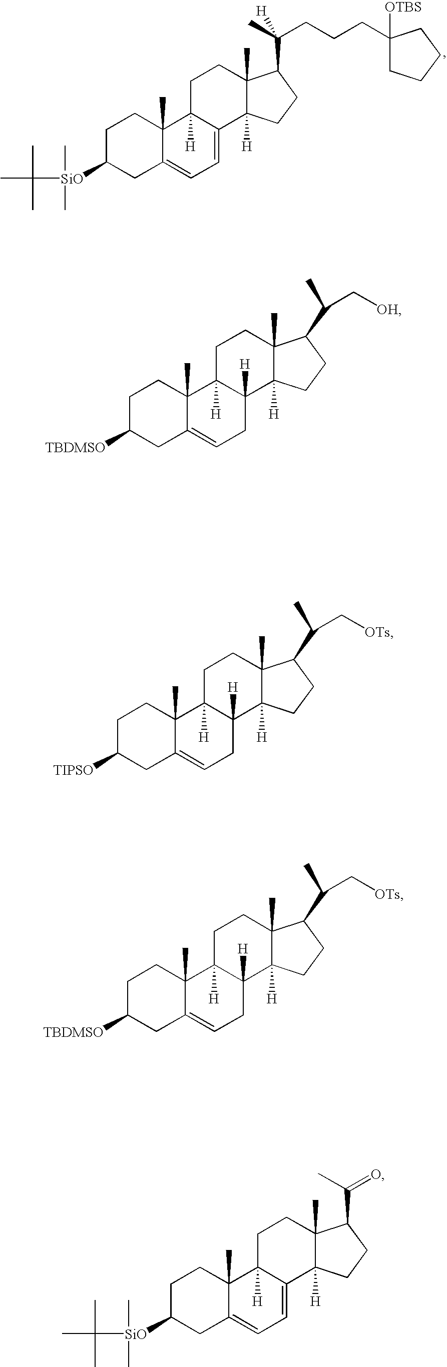

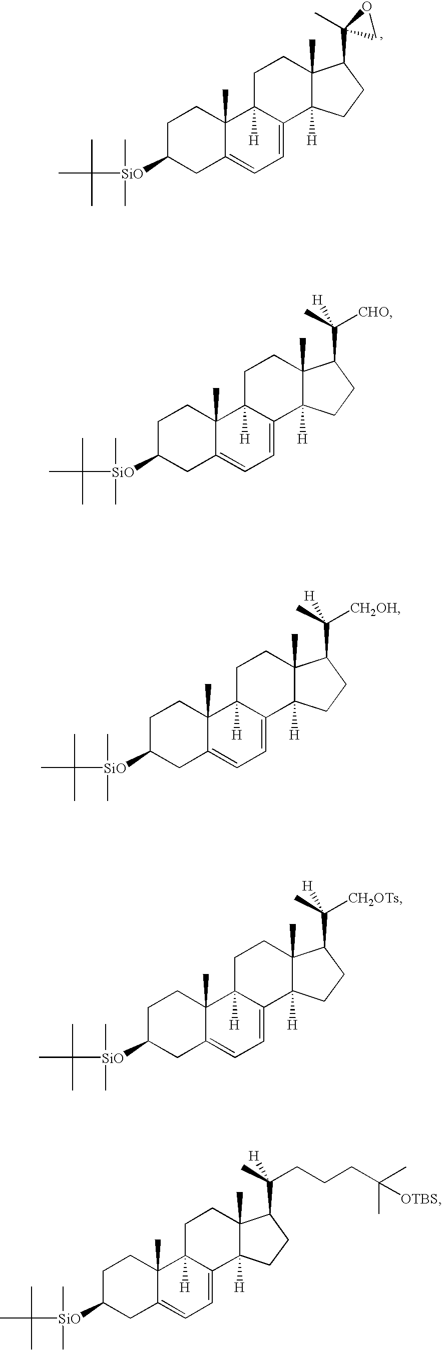

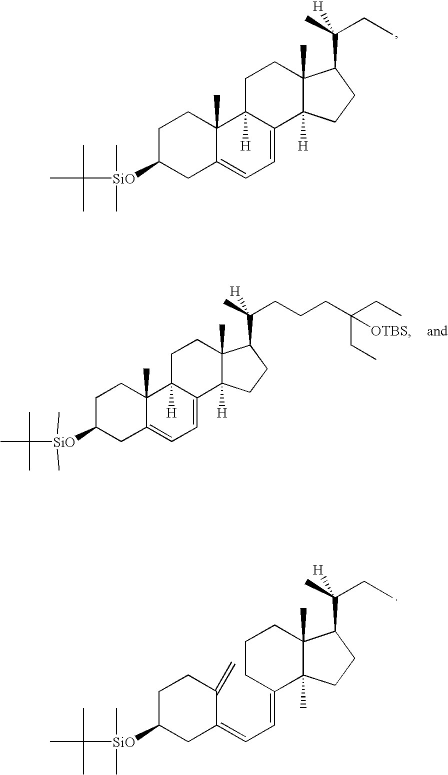

These compounds may be used to make the compounds of disclosed in this paper.

The methods of the first and second aspects may be used to make the compounds of the formulas:

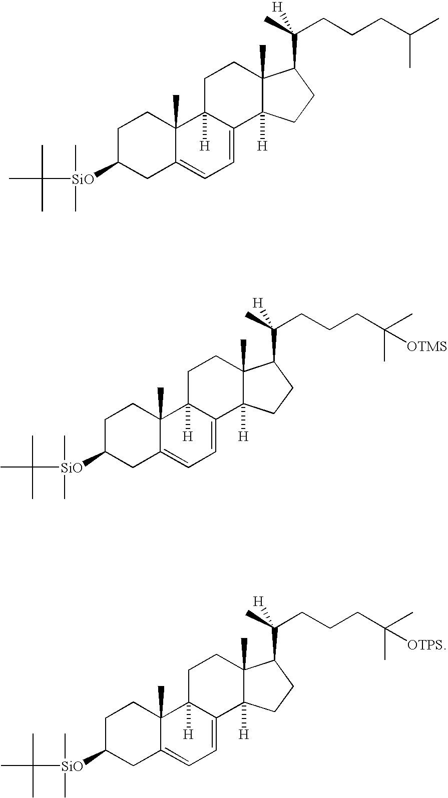

The methods of the first and second aspects may be used to make the compounds of the formulas:

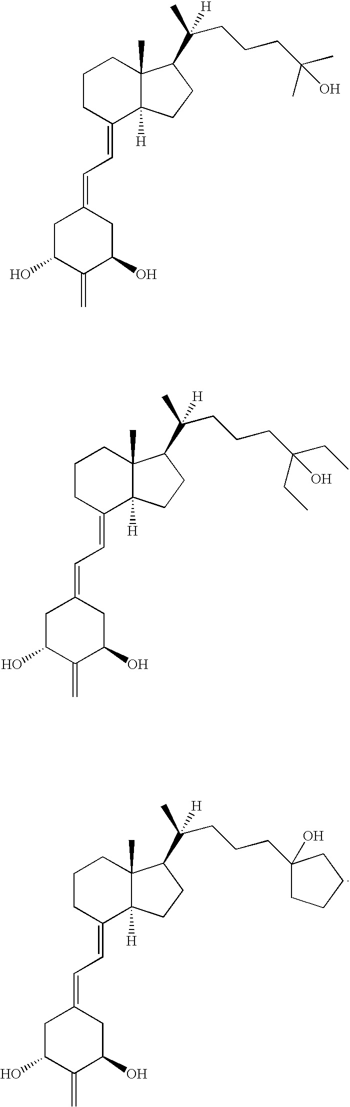

The methods of the first and second aspects may be used to make the following compounds:

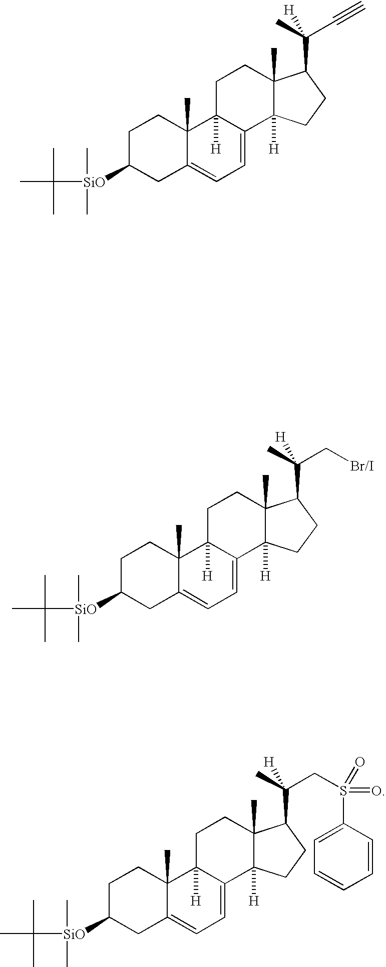

The methods of the first and second aspects may be used to make the compounds of the formulas:

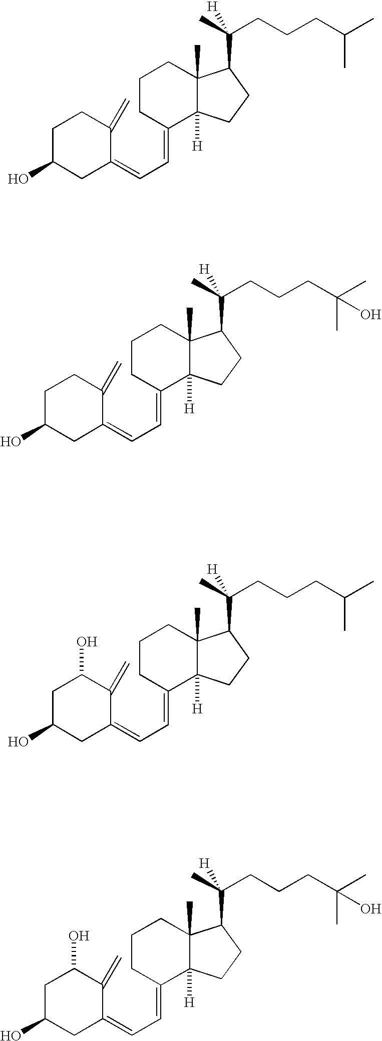

The methods of the first and second aspects may be used to make the compounds of the formulas:

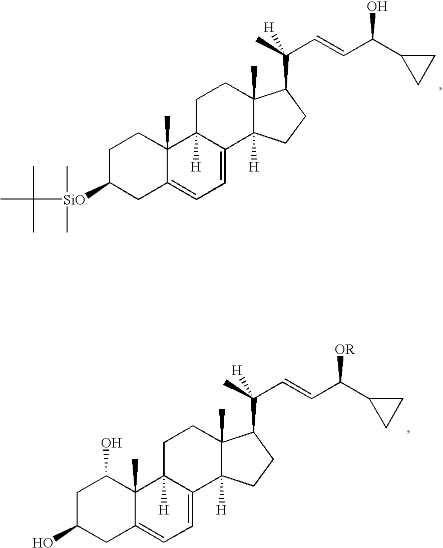

where R═H, TMS, MEM, TPS, TBDMS, or

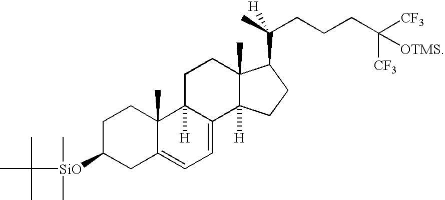

One of skill in the art will appreciate that the TBS groups (above) may be replaced with a TIPS group and that the TMS group may be replaced with TBS, TES, MEM, or C₁-C₆ alkoxy.

The methods of the first and second aspects may be used to make the compounds of the formulas:

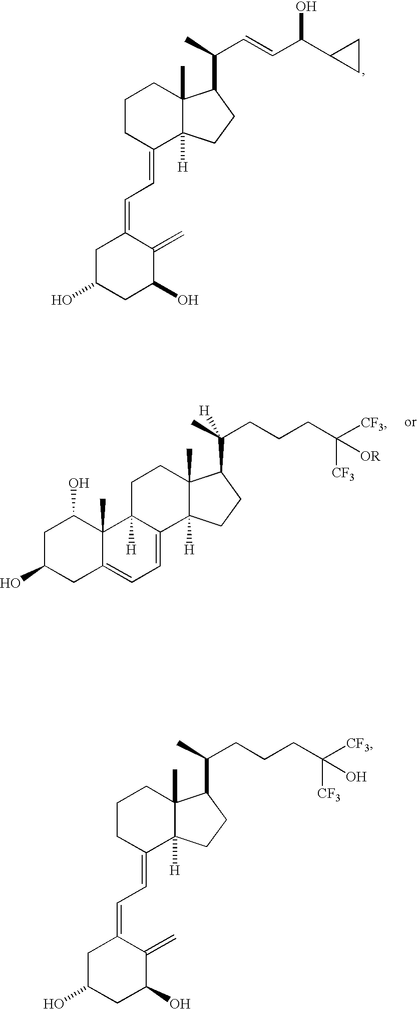

where R═H, TMS, MEM, TBDPS, or TPS.

The methods of the first and second aspects may be used to make the compounds of the formula:

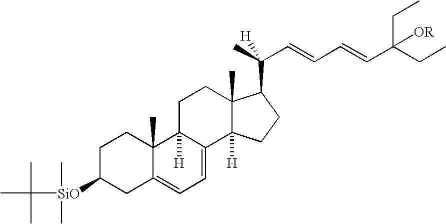

where R═H, pivaloate, TMS, MEM, TBDPS, or TPS.

The methods of the first and second aspects may be used to make the compounds of the formula:

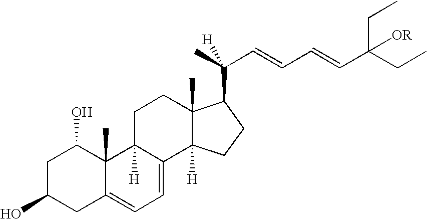

where R═H, pivaloate, TMS, MEM, TBDPS, or TPS.

The methods of the first and second aspects may be used to make the compounds of the formulas:

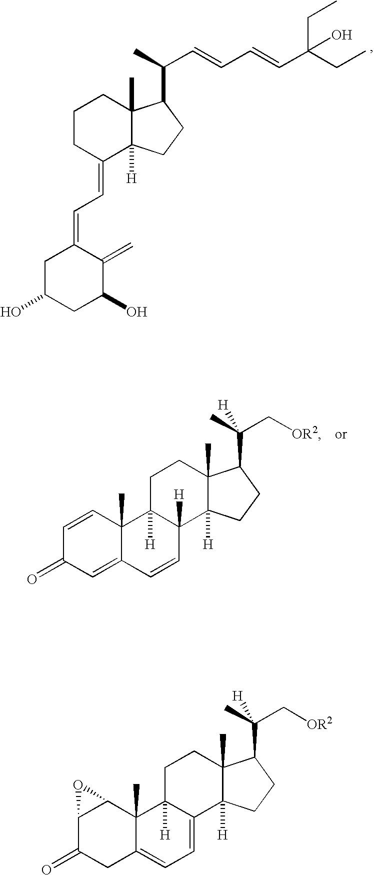

where R²=TMS, Trityl, TBDMS, pivaloyl, TPS, TIPS, TBDPS, or other alcohol protecting groups known in the art and where R₂ may also be H.

The methods of the first and second aspects may be used to make the compounds of the formula:

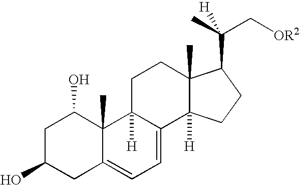

where R²=TMS, Trityl, TBDMS, pivaloyl, TPS, TBDPS, or other alcohol protecting groups known in the art and where R₂ may also be H.

The methods of the first and second aspects may be used to make the compounds of the formula:

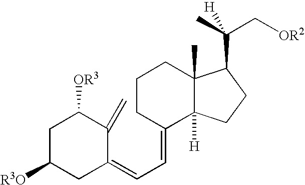

where R² and R³ are different, and drawn from the group; H, TMS, Trityl, TBDMS, pivaloyl, TPS, TBDPS, or other alcohol protecting groups, in such a combination that R² can be removed in the presence of R³, which are known in the art.

The methods of the first and second aspects may be used to make the compounds of the formula:

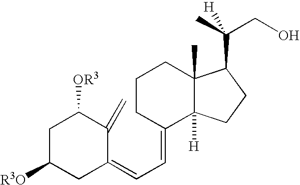

where R³=TMS, acetate, TBDMS, pivaloyl, TPS, TBDPS, or other alcohol protecting groups known in the art and where R₃ may also be H.

The methods of the first and second aspects may be used to make the compounds of the formula:

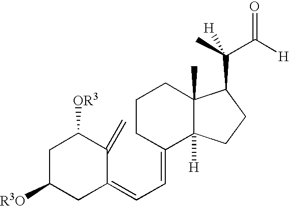

where R³=TMS, acetate, TBDMS, pivaloyl, TPS, TBDPS, or other alcohol protecting groups known in the art and where R₃ may also be H.

Further disclosed are pharmaceutical compositions comprising steroids and Vitamin D derivates made using the method of the first or second aspects and at least one pharmaceutically acceptable carrier, excipient, adjuvant or glidant.

Further disclosed are pharmaceutical compositions comprising the following compounds:

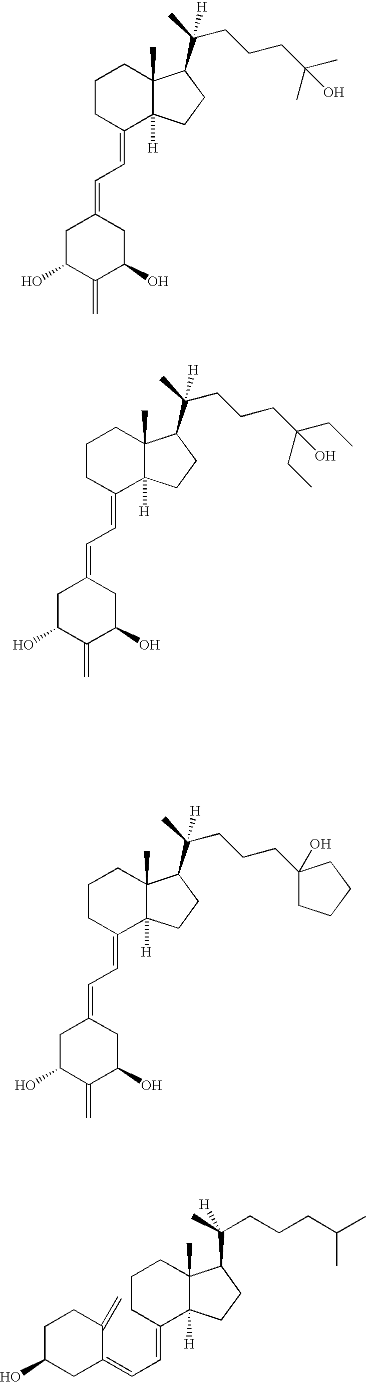

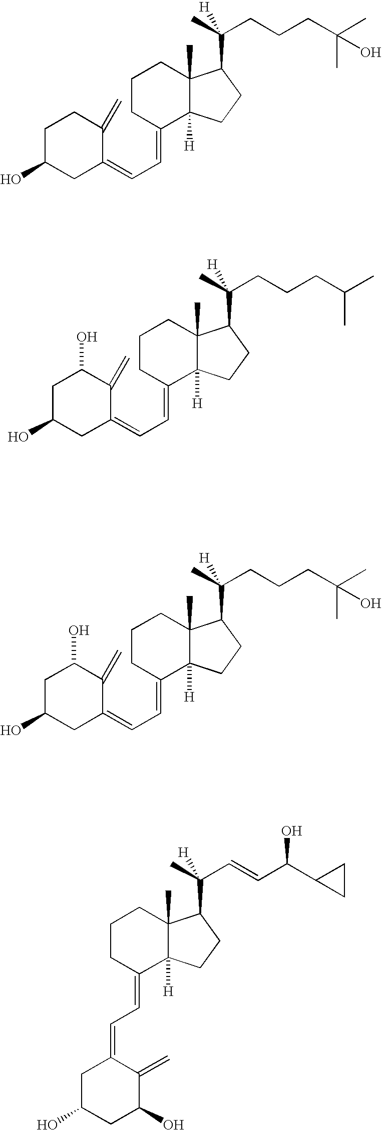

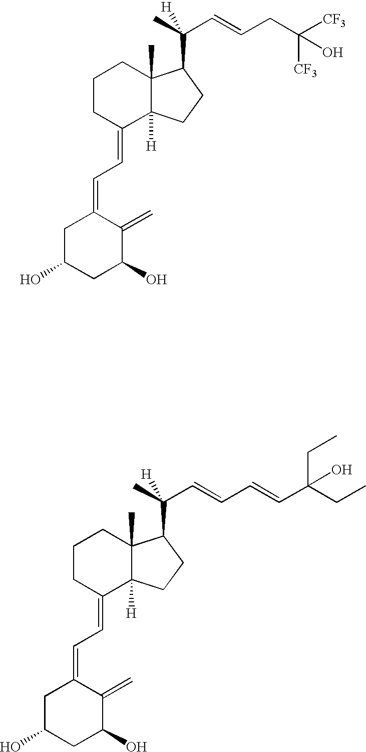

and at least one pharmaceutically acceptable carrier, excipient, adjuvant or glidant.

The methods of the first and second aspects may be used to make the compounds of the formula X:

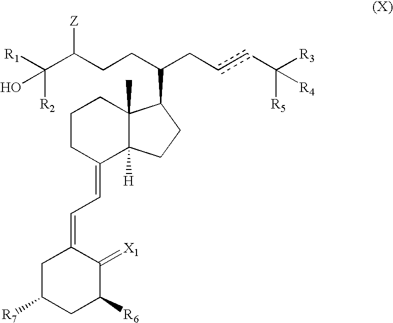

wherein:
the C23-C24 bond may be a single, double or triple bond;
R₁, R₂, R₃ and R₄ are each independently C₁-C₄ alkyl, C₁-C₄ deuteroalkyl, hydroxyalkyl or haloalkyl;
R₅, R₆ and R₇ are each independently OH, OC(O)C₁-C₄ alkyl, OC(O)hydroxyalkyl or OC(O)haloalkyl;

### X₁ is CH₂;

### Z is H, OH, ═O, SH or NH₂

The methods of the first and second aspects may also be used to prepare compounds of formula X, wherein R₇ is OH, and R₁, R₂, R₃ and R₄ are each independently C₁-C₄ alkyl, hydroxy C₁-C₄ alkyl or C₁-C₂ haloalkyl.

The methods of the first and second aspects may also be used to prepare stereospecifically at C20 compounds of formulas:

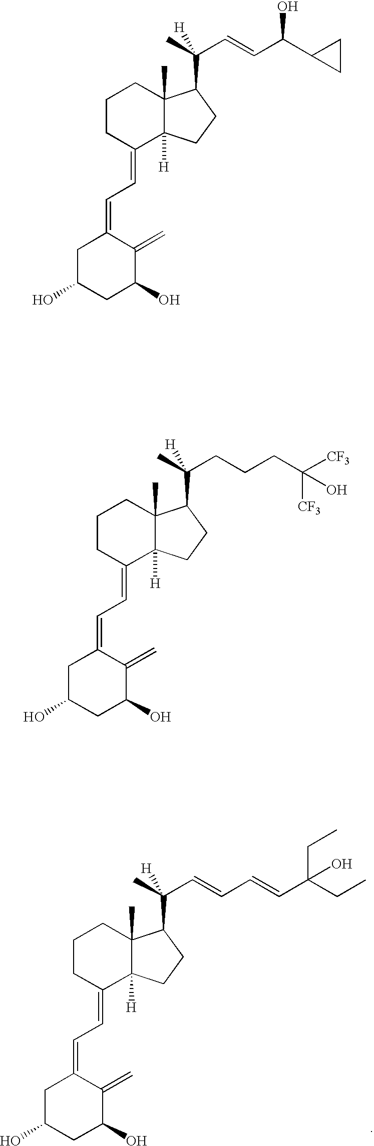

### DEFINITIONS

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. The aryl groups herein are unsubstituted or, as specified, substituted in one or more substitutable positions with various groups. Preferred examples of aryl groups include phenyl, naphthyl, and anthracenyl. More preferred aryl groups are phenyl and naphthyl. Most preferred is phenyl.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, and dihydropyrrolidinyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidine. Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl. More preferred heteroaryl rings include pyridyl, pyrrolyl, thienyl, and pyrimidyl.

### A. Hydroxyl Protection of Pregn-5-en-3β-ol-20-one

As described above, in one aspect the invention provides the use of pregnenolone (1) to produce O-protected 20R,22-homopregn(adi)en-22-als (2 & 4) and O-protected 20R,22-homopregn(adi)en-22-ols (3 & 5) derivatives in good overall yield, and high diastereomeric purity at C20. Generally, the alcohol protecting groups described in Protecting Groups in Organic Synthesis by Greene, may be used in this process if compatible with the next two steps, but in a preferred aspect, the protecting group, PG, is a silyl protecting group. Both the t-butyldimethylsilyl (TBDMS or TBS) ether (7) and the triisopropylsilyl (TIPS) ether (8) are especially preferred and are relatively inexpensive. Moreover, many other protecting groups, especially other silyl ethers such as t-butyldiphenylsilyl (TBDPS) and phenyldimethylsilyl (PDMS), are also useful. While still useable, ester protecting groups tend to be cleaved by the preferred nucleophilic epoxidizing agent used in the key step to set up C20 stereochemistry, and further limit the chemistries which may be used to elaborate key intermediates (4) and (5). The TBDMS ether (7) was obtained in excellent purity and 98% yield by direct crystallization from the reaction mixture. TIPS ether (8) was not quite as easy to obtain, and yet was obtained in 84% yield after recrystallization, or about 90% yield after column chromatography, and these protecting groups proved very satisfactory when the C17 side chain was introduced first, as shown in Scheme 1. However, when the 7,8-unsaturation was introduced first, the preferred base proved to be fluoride ion (see below), and for both cost and convenience, pregnenolone acetate (9) was used as the starting material, and a switch was made to the TBDMS ether at a later stage in the synthesis. Pregnenolone acetate can be made from pregnenolone in above 99% yield, or bought commercially. Other protecting groups which may be used at the 3-hydroxy include methyl (produced by solvolysis from the corresponding sulfonates), benzyl and allyl (which may be derived from the corresponding O-substituted trichloroacetimidates).

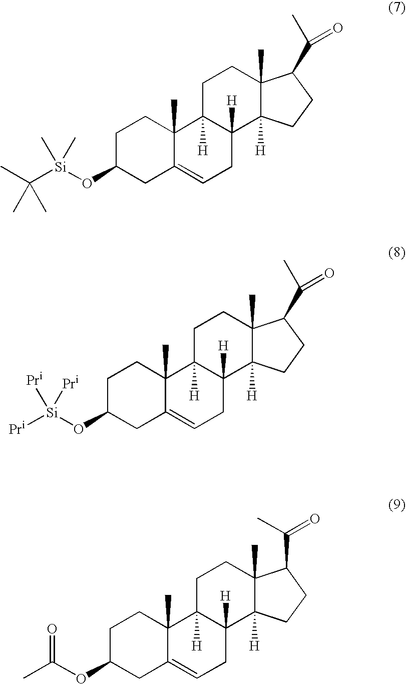

### B. Introduction of the 7,8-Double Bond to Pregn-5-en-3β-ol-20-one and 22,23-Bishomopregn-5-en-3β-ol Derivatives

The O-protected pregnenolone derivatives, (7-9) described above can all be allylically brominated by a variety of brominating agents at the 7-position to give bromides (10), as described in the literature. Numerous bases are described to dehydrobrominate (10) to the corresponding protected dienone (11). As this transformation is usually described for the conversion of O-protected cholesterol derivatives into 7-dehydrocholesterol derivatives, it should be especially suitable to the conversion of protected 22,23-bishomopregn-5-en-3β-ol derivatives (12) to the corresponding bromides (13), which can then be eliminated to the desired diene (14).

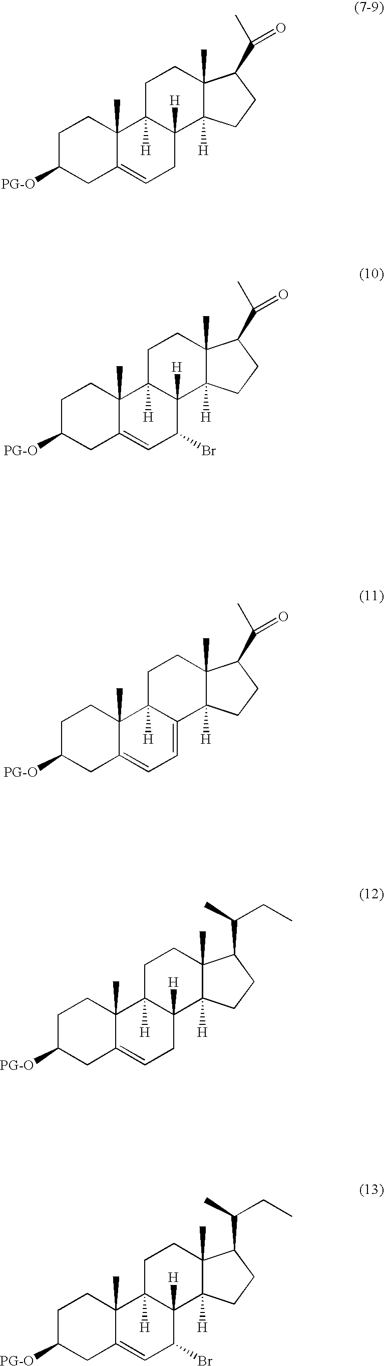

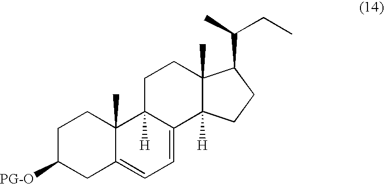

This reaction sequence has three major drawbacks. The first is that the 7α-bromide is the only one set up to eliminate properly, that is transdiaxially to H8β, and bromination of different steroids can give very variable 7α/β mixtures, sometimes with the unwanted equatorial β-isomer predominating. The use of a soluble bromide source (such as tetra-n-butylammonium bromide (TBAB)) in a suitable solvent equilibrates the two bromides, and such equilibria generally favour the desired α-(axial) isomer by 2.5-4:1 ratios, ameliorating this problem considerably. This problem is exacerbated by the fact that these α/β mixtures of bromides are often very difficult to reliably quantitate, even by highfield proton nmr.

The second problem is that a lot of the literature describing these reactions is very old, and the analytical techniques used did not always distinguish the desired 5,7-diene product, a product of an expected trans-diaxial 1,2-elimination, from the unexpected trans-diaxial 1,4-elimination, which leads to the unwanted 4,6-diene. Molecular modeling shows that the 8β-proton, which is the proton extracted in the desired 1,2-elimination, is considerably more hindered by the β-methyls C18 and C19 than is the 4β-proton, abstraction of which leads via 1,4-elimination to the 4,6-diene. We have found literature reaction conditions which can produce almost exclusively the 4,6-diene when applied to some steroidal precursors. Other side products were often not detected in the older literature, and often they cannot be reliably removed by crystallization, or chromatography.

The third problem is that the allylic bromides (10, 13) are rather unstable, and the range of reagents and solvents usable with the 7-bromides is very limited. For example, the bromides cannot be purified by normal phase silica gel chromatography, and the base/solvent combinations to do the 7,8-elimination are rather limited. This is especially true for pregnenolone derivatives, which have a tendency to epimerize at C17, and/or enolize at C21, when treated with very strong bases. We examined a variety of bases on 7-bromopregnenolone derivatives, and found that many bases induced no elimination under conditions close to causing carbonyl-related problems, or when they did eliminate, there were unacceptably high, sometimes even major, amounts of the 4,6-dienes produced. In fact this latter point led to the development by Confalone et al. (Confalone, P. N., Kulesha, I. D., Uskovic, M. R. J. Org. Chem. (1981), 46, 1030-2.) of a three step conversion of the 7α-bromide into the corresponding 5,7-diene, which involves displacement of the bromide by an aryl thiol, to form a thioether, oxidation of said thioether to the corresponding sulfoxide, and a pyrolytic sulfoxide elimination to form the diene specifically in the 5,7-position. This four step reaction sequence can work in around 50% yield, and produce very clean 5,7-dienes. This is towards the upper end of reported yields for sequences involving a direct bromination-dehydrobromination, which generally work in 35-50% overall yields. We have found that this sequence works reasonably well in a Scheme 1 based preparation of 20S,3β-(triisopropylsiloxy)-22,23-bishomopregna-5,7-diene, (15, (14, PG=TIPS)), converting the corresponding monoene (16, (12, PG=TIPS)) into (15) in up to overall 50% yield, as illustrated in Scheme 3. However, the initial bromination to make (17) is difficult to monitor, and highly reproducible conditions for pushing the reaction to completion were not found. The 7α:7β bromination ratio appeared to be rather unfavorable, although the crude nmrs generally look as though they contain predominantly a single isomer. However, direct reaction of the crude bromide with 4-chlorothiophenol gave a complex mixture, where the major component is not the same as that seen if a TBAB equilibration step is included, and where the desired β-thioether (18) is clearly not the major species present. Thiol displacement, after TBAB equilibration, as demonstrated by an axial H7-proton at 3.31δ, with an 8.5 Hz coupling constant, gives the β-thioether (18) in good yield with only 10-15% of the unwanted α-isomer being present. Oxidation to the sulfoxide (19) could be carried out satisfactorily with mCPBA, although both diastereoisomeric sulfoxides were produced, as described by Confalone. The thermolysis to (15) went smoothly, although again as described by Confalone, the minor diastereoisomeric sulfoxide decomposes a lot more slowly than the major one. However, removal of the disulfide byproducts, and unreacted (16) proved very difficult. Because this double bond introduction involves the lowest yielding reactions in the entire sequence, it was decided to examine carrying it out earlier, where comparable material losses should be less costly.

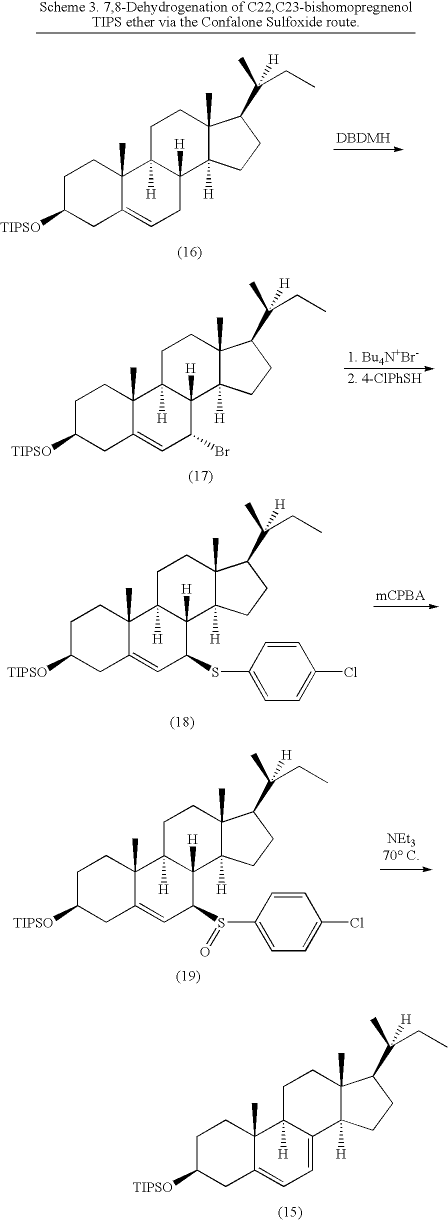

In order to make the overall process more cost effective, we examined the allylic C7 bromination of pregnenolone derivatives, with the intention of following a Scheme 2 sequence, whereby the 7,8-double bond was introduced prior to C17 side chain elaboration. One can envision using this sequence on a silyl-protected pregnenolone derivative such as TBDMS-pregnenolone (7), to produce the most desired O-silylpregnadienone derivative (20, (11, PG=TBDMS)) (see below). Literature on the bromination of pregnenolone derivatives is very sparse, but a bromination-dehydrobromination sequence on pregnenolone acetate (9), which works in around 50% yield has been described (Siddiqui, A. U., Wilson, W. K., Swaminathan, S., Schroepfer, G. J. Chemistry and Physics of Lipids, (1992), 63, 115-129).

We have examined the sequences, shown in Schemes 4 and 5, in order to introduce the 7,8-double bond early in the sequence. Although the desired final product from this sequence for the 20-epi derivatives is the silyldienone (20), the shortest route involving the bromination of silylether (7), followed by base-induced dehydrobromination was not deemed practical, as the only base we found which produced a high enough 5,7- over 4,6-diene selectivity was fluoride ion, which also removes the TBDMS group. Thus the product will be the free dienol (21), which would have to be resilylated to make (20). This not only introduces an extra step, but it also means doing two protections with a rather expensive protecting group, TBDMS chloride, and it uses up an extra equivalent of the rather expensive base TBAF. Therefore, we examined the Confalone procedure with silyl ether (7), and chose to examine the acetate (9) with the base-induced double bond introduction, as the acetate is very cheap, easy to put on, and will not require extra fluoride in the elimination. However, the need to change protecting groups does add two extra steps, even if the yields are very good.

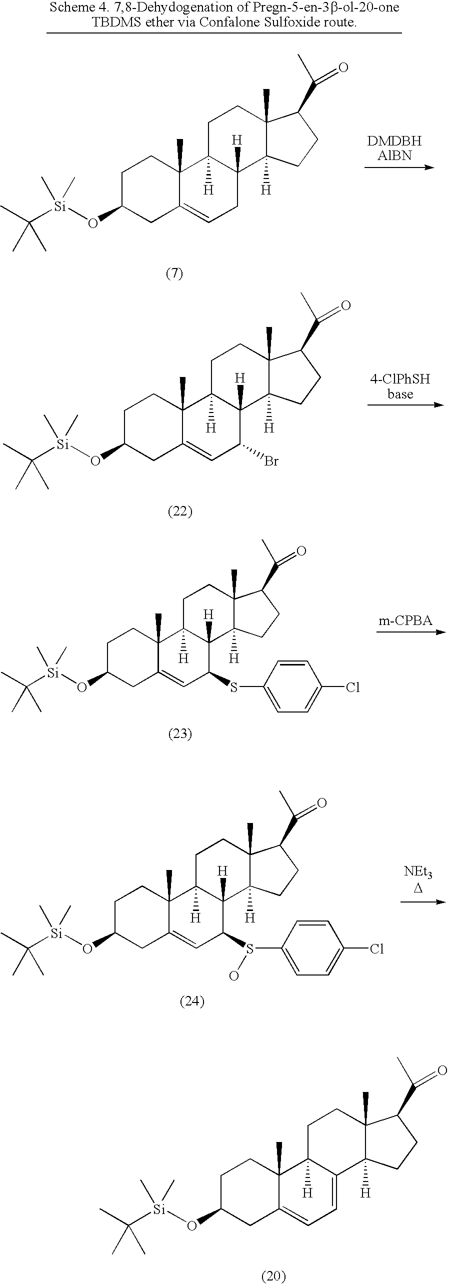

Bromination of silylpregnenolone (7) with 1,3-dibromo-5,5-dimethylhydantoin (“Bromantin”, “DMDBH”) went smoothly, afforded a relatively clean 7-bromide product assigned as (22). Although the product is not stable to thin layer chromatography (tlc), and shows multiple spots, all major ones are slower than (7), allowing reaction completion to be monitored. NMR analysis of the crude reaction mixture is suggestive that one isomeric bromide greatly predominates, and that the second isomer, if present at all, is one of several minor (<10%) impurities. As discussed above, this apparent selectivity was also seen with the bromination of (16), but did not appear to reflect the true ratio, which was worse than 1:1. However, in this case, the “Confalone” analysis, done once the bromide had been displaced by a thiol, but without any form of bromide equilibration, suggests that the 7α:7β bromide ratio is usually >10:1, which is at least as good as one would get after equilibration. Although this crude mixture appears to be quite clean by nmr, carrying it on without purification at this step led to lower overall yields than expected. Both attempts to purify the sulfide, or to carry the crude mixture through the remaining reaction sequence to diene (20), led overall to lower yields than expected, and best yields of (20) from (7) were around 35%. Therefore, crystallization of bromide (22) was examined. The crude product tends to partially solidify, but simple recrystallization tends to give less than 50% yield of (22). However, careful examination of crystallization conditions allowed for bromide (22) to be isolated in 65% yield in over 90% purity. Reaction of this bromide with 4-chlorothiophenol led to the sulfide (23) in 92.7% yield. This could be oxidized to a diastereoisomeric mixture of sulfoxides (24) in 82% yield, and this in turn yielded the diene (20) in 80.6% yield after a gentle pyrolysis at 70° C., in the presence of triethylamine, for an overall yield of 40% from (7).

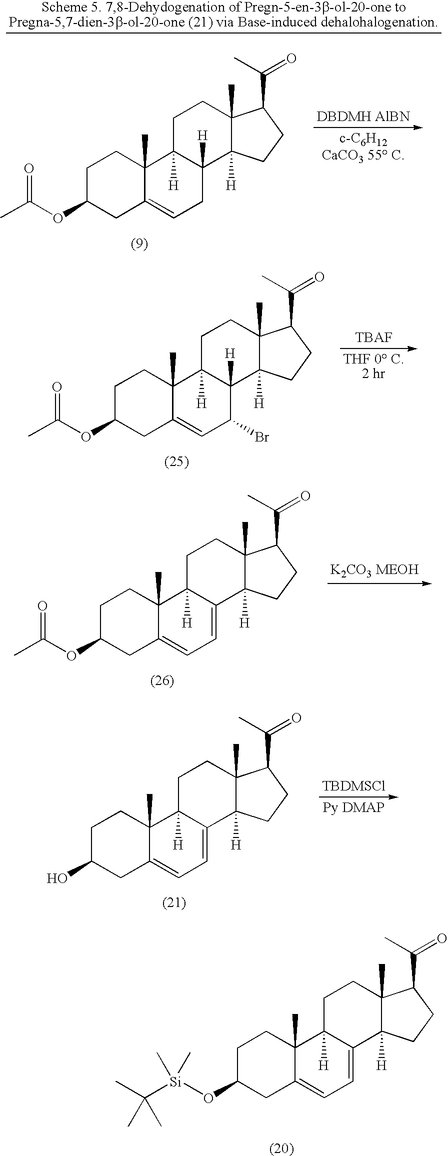

A study of the bromination of pregnenolone acetate (9) demonstrated that it is also readily brominated at the 7-position by 0.65 molar equivalents of Bromantin in degassed cyclohexane with moderate heating (55-75° C.) to form mainly 7α-bromopregnenolone acetate (25) as reported by Siddiqui et al. (Siddiqui, A. U., Wilson, W. K., Swaminathan, S., Schroepfer, G. J. Chemistry and Physics of Lipids, (1992), 63, 115-129). NMR spectra of the crude reaction products suggest that this product is formed in 85-90% yield, with very little of the unwanted 7β-bromide. NMR analysis of the thiol displacement product(s) also indicates a 7α:7β ratio of at least 10:1. Again the instability of the bromide product (25) to silica gel, makes analysis of the reaction by tlc difficult, but it does allow one to monitor for the disappearance of starting material reliably. Once the reaction is essentially complete by tlc, the reaction mixture is filtered hot to remove unreacted dibromantin and the 5,5-dimethyl hydantoin side product. This solution can be stripped to dryness to give the bromide (25) as a solid white to light yellow foam in crude quantitative yield, which appears to be 85-90% pure by nmr spectroscopy. As with the TBDMS ether, use of this material crude led to much lower yields than expected in later steps, and it was also found advantageous to crystallize bromide (25). As the reaction mixture is concentrated to the 0.5-1.0 M range under reduced pressure, 7α-bromopregnenolone acetate (16) of 95-99% purity starts crystallizing out. However, this process does not produce much above 50% of (25), and further crystallizations of the mother liquors are required to get the yields of (25) up to 68-75%.

The tetra-n-butylammonium fluoride (TBAF) induced dehydrobromination reaction on 7α-bromopregnenolone acetate (25) as described by Siddiqui et al. (Siddiqui, A. U., Wilson, W. K., Swaminathan, S., Schroepfer, G. J. Chemistry and Physics of Lipids, (1992), 63, 115-129) was examined. Treatment of recrystallized 7α-bromopregnenolone acetate (25) with three equivalents of TBAF solution in THF at temperatures between 0° C. and reflux, for times between five minutes and three hours leads to complete loss of the starting material. Depending on the quality of the starting bromide and the TBAF solution, which appears to be mainly a question of how dry the solution is, pregna-5,7-dien-3β-ol-20-one acetate (27) is obtained in 70-98% purity, and 90-96% crude yield. For use in making 20-epi-Vitamin D derivatives, the acetate group does not appear to be as desirable as using silyl ether protecting groups. Therefore the acetate group needs to be cleaved, which can be done in very high yield with methanol and catalytic solid potassium carbonate to give pregna-5,7-dien-3β-ol-20-one (21). This route is shown in Scheme 5, and results in overall yields of (21) from pregn-5-en-3β-ol-20-one (1) of 50-65%.

A very useful extension of this methodology is revealed herein, whereby the elimination and deesterification steps are combined together. Thus, upon completion of the TBAF elimination reaction, the reaction mixture is treated with at least an equal volume of methanol, and a molar excess of potassium carbonate over the originally added TBAF. After a few hours stirring this mixture at 25° C., the reaction can be quenched with excess ice-water, and the crude pregna-5,7-dien-3β-ol-20-one can be collected in 90-95% overall yield by a simple Buchner filtration. The material obtained is of about 90% or better purity, and can be used without purification.

Although acetate (26) is not useable in the chemistry described below, and alcohol (21) can only be used in said chemistry after being suitably protected, these two compounds are useful intermediates in a wide variety of other steroid/Vitamin D syntheses, as they combine a B-ring diene and a readily modified C17 side chain, and are obtained in very few steps, and good overall yields from pregn-5-en-3β-ol-20-one (1). Pregna-5,7-dien-3β-ol-20-one (21) can be protected on the alcohol oxygen using many different protecting groups, as described in Protective Groups in Organic Synthesis 3^{rd} Edn. by Greene and Wuts. The B-ring 5,7-diene system can be modified in many different ways, especially oxidatively to produce a wide variety of biologically active steroids with highly functionalized, or even cleaved B-rings.

Silylation of pregna-5,7-dien-3β-ol-20-one (21) can be carried out conveniently with t-butyldimethylsilyl chloride and pyridine with DMAP catalysis in DMF in the temperature range 25-55° C. By running this reaction rather concentrated, the desired product, 3O-(t-butyldimethylsilyl)pregna-5,7-dien-3β-ol-20-one (20) precipitates in good yields, 80-93%, and with a considerable increase in purity over the starting alcohol. If the starting alcohol is >90% pure this allows for the product to be obtained directly from the reaction mixture in >98% purity, which is adequate for the succeeding chemistry without need of further purification.

### C. Introduction of the C17-[S],2-Butyl Side Chain to Pregn-5-en-3β-ol-20-one and Pregna-5,7-dien-3β-ol-20-one Derivatives

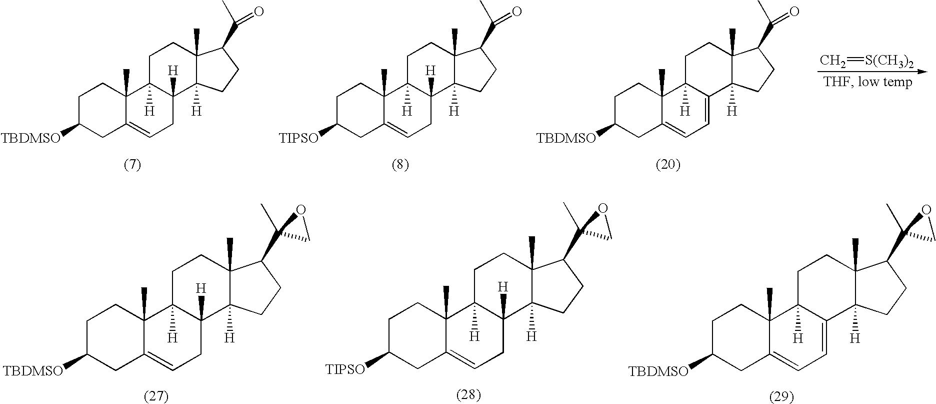

The 3-THP ether of pregnenolone is reported to react with dimethylsulfonium methylide in DMF at room temperature to produce the corresponding 20[S]-epoxide (Koreeda, M.; Koizumi, N. Tetrahedron Letters, 19, 1641-4, (1978)). We examined this reaction by nmr, and found that it appears to have a diastereoselectivity of around 19:1 for 20[S]:20[R]. However, it is difficult to be confident of that ratio, as the THP itself introduces an uncontrolled chiral center. This reaction has two further disadvantages. Both the steroid and the ylide are sparingly soluble in DMF, and the reaction is very slow, taking up to a week to go to completion. This requires a very concentrated reaction mixture, and one ends up with a thick paste, which is difficult to stir even on a small scale.

To overcome this problem, we examined several different protected pregnenolone derivatives, different solvents, and increasing the reaction temperature. Apart from a slight improvement by using N-methylpyrrolidone, all other solvents examined failed to improve the reaction, dilution slowed the reaction drastically, and heating led to predominant production of unwanted side products. The only 3-derivative which gave comparable results to the THP-ether was the methoxyethoxymethyl (MEM) ether, and this confirmed the diastereoselectivity ratio at C20 to be around 15:1. Most other 3-derivatives were either cleaved (most esters) by the ylide, or reduced the solubility of the steroid in DMF and NMP so much that virtually no reaction occurred.

Most nucleophiles do not attack the carbonyl of pregnenolone with a very high diastereofacial selectivity, so the good diastereoselectivity of the sulfonium ylide attack is on the face of it rather surprising. However, dimethylsulfonium methylide is a rather stable anion, and its addition to ketone carbonyls is generally reversible. This means that the reaction can come under thermodynamic, rather than kinetic control, but one would not expect the final diastereoisomeric epoxides to differ appreciably in stability. However, when one examines the rather rigid transition state, required to convert the intermediate betaine into the corresponding epoxide, it becomes evident that the transperiplanar geometry required for the alkoxide, and dimethylsulfonium leading groups can only be acconmodated in a single conformation. In this conformation, the transition state for the minor [S]-epoxide has a severe steric clash between the C18 and C21 methyl groups, whereas the [R]-epoxide transition state avoids this interaction completely. This suggests that the diastereoselectivity arises because only the [R]-epoxide forming transition state is readily attainable, and carbanion addition from the si-face attack is likely to reverse more readily than it is to go to the epoxide thermodynamic sink.

As dimethylsulfonium methylide is rather less stable, and hence a more reactive anion, than its sulfonium analogue, one would expect the initial carbonyl addition to be less readily reversed, and consequently, one would expect the diastereoselectivity to be more affected by the initial nucleophilic attack, and hence rather poorer. Surprisingly, when we examined the reaction of 3-tetrahydropyranylpregnenolone with dimethylsulfonium methylide at room temperature in THF, the diastereoselectivity of epoxide formation was almost as good as was seen with the sulfonium ylide.

The protected alcohol-ketones (7), (8) and (20) were also converted into the epoxides (27)-(29) using the ylide derived from triimethylsulfonium iodide. A wide variety of strong bases, obvious to one skilled in the art will produce this ylide from trimethylsulfonium iodide or bromide, exemplified by, but not limited to, potassium hexamethyldisilazane. These reactions were complete in 10 minutes at room temperature in THF and the reactions were homogenous solutions, with some salt precipitation, without any of the stirring problems seen with the sulfonium ylide. The surprisingly good diastereoselectivity seen with the THP derivative was also seen in these cases, and these reactions, for which no workable conditions were found at all with dimethylsulfonium methylide, were simple to do and very high yielding.

Upon using the TBS or TIPS protecting group and lowering the reaction temperature, the reaction between the ylide derived from dimethylsulfonium iodide and the TBS or TIPS protected pregn(adi)enolone affords a product that is increasingly clean, diastereoselective, and high yielding. For example, in a dry ice-isopropanol bath, epoxides (27)-(29) are produced with diastereoselectivities in the 40-55:1 range, and yields above 90% with overall reaction times of a few hours. The rather poor low temperature solubility of these substrates in ethereal solvents makes the use of a cosolvent, preferably toluene, essential for this reaction to run well. Furthermore, if desired, the epoxides can be recrystallized to much higher diastereomeric purities, using solvents obvious to one skilled in the art. For example a C20 R:S ratio in of the range of 200:1 was obtained after a single recrystallization from acetone at 0° C., in an overall 75% yield for epoxide (27). However, despite the excellent diastereoselectivities available after recrystallization, it appears to be most advantageous to accept the high crude yields in this step, and to purify compounds later in the sequence. Due to major differences in the chemical shifts of the C22 (epoxide) protons, and the C18-methyl protons between the two diastereoisomers, their ratios are readily determined by nmr to better than 0.5% accuracy.

Before converting protected alcohol-ketones (7), (8) and 20) into epoxides (27), (28) and (29), the C21-methyl side chain may be elaborated by generating a kinetic enolate via C21 proton abstraction, using a base, such as LDA, NaHMDS, KHMDS or others as known in the art in a solvent, such as THF, usually at low temperature, and then reacting the enolate with an electrophile as shown in Scheme 6. (Konopelsli, J. P., Djerassi, C. J. Med. Chem., 23, 722-6, (1980).) Examples of such reactions include, enolate alkylation, directed Claisen reactions, the directed aldol reaction, the Mukaiyama aldol reaction, the Michael reaction and others. The resulting compound may then be converted diastereoselectively into the C20-C22 epoxide as described above, and further elaborated at C22, as described below.

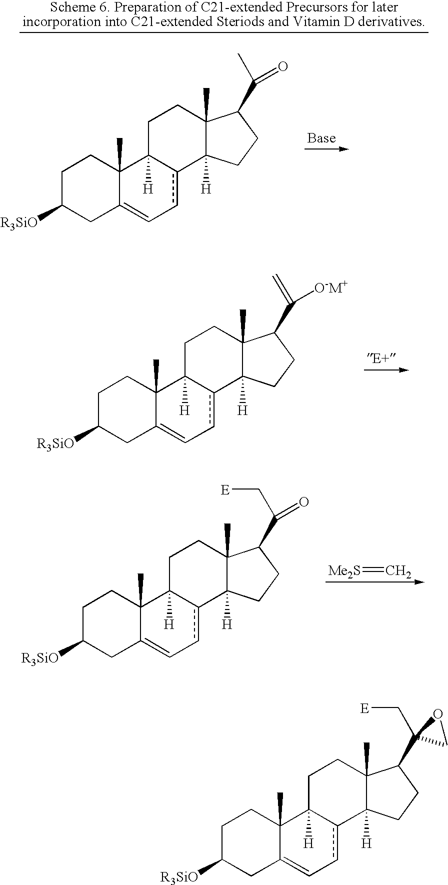

In scheme 6, the R group may be the same or different and is selected from methyl, ethyl, isopropyl, tert-butyl and phenyl. Preferred R₃Si groups include TBDMS, and TIPS.

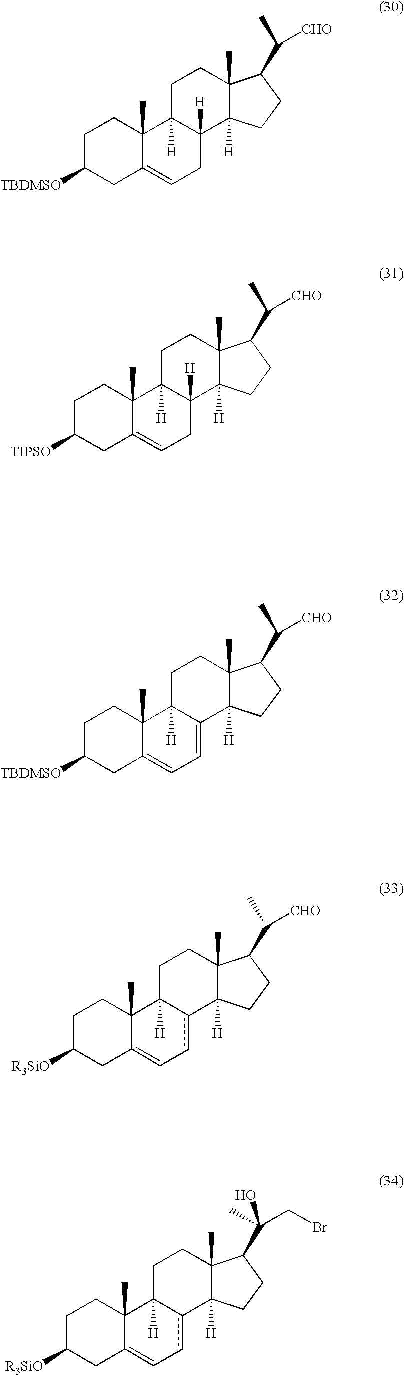

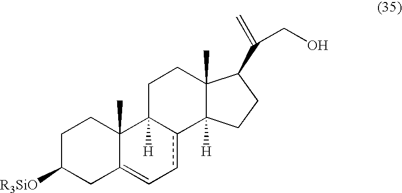

The conversion of the epoxides (27)-(29) to aldehydes (30)-(32) is performed using a Lewis acid. This reaction is neither stereospecific nor chemospecific, and at least three products other than the 20-R aldehyde are produced in this reaction, regardless of which epoxide is used. The undesired S-aldehyde (33) is present as 2-45% of the mixture, and simple halide induced S_{N}2 opening of the epoxide to form a halohydrin (34) consumes 0.5-15% of the epoxide, and an apparently base-induced epoxide opening to 1-10% of an allyl alcohol (35) also occurs. A wide variety of Lewis acids have been examined for this transformation in the monoene series; BF₃ etherate, BCl₃, MgCl₂, MgBr₂, MgI₂, Al(OPrⁱ)₃, Ti(OPrⁱ)₄, titanocene dichloride, ZnCl₂ etherate, GaCl₃, and In(OTf)₃. Additionally, various Lewis acidic reagents, which should cause the epoxide to rearrange to the aldehyde, and then react with the aldehyde in situ were also examined in the monoene series; MeMgBr, TMSCH₂MgCl, TMSCH₂MgBr, BH₃/BF₃, BH₃/BCl₃, Tebbe reagent, Petasis reagent, and DIBAL-H. Almost all of these reagents gave the desired products, and often in good overall yields, but none were judged stereoselective enough to be used preparatively, with DE's of ˜33-85% being obtained. The optimal Lewis acid for this transformation was found to be magnesium bromide, used as the solid bis-diethyl etherate. This was then optimized for solvent, stoichiometry, and temperature. The optimal conditions for all three epoxides (27), (28) and (29) were found to be with toluene as solvent, 0.2-0.5 equivalents of the Lewis acid, and temperatures in the −10 to 0° C. range, which 1) consistently afforded a C20 R:S ratio of 25:1 or better, and 2) reduces the production of the byproducts to about 5%. We have found that C20 R:S diastereomeric ratios of 15-20:1 can be obtained using unpurified epoxides (27)-(29) in the reaction mixture, and that the diastereomeric purity of the product can be raised up to about 65:1 20R:S for TBDMS aldehyde (30), and 35:1 for TIPS aldehyde (31) after a single recrystallization from acetone or isopropanol respectively in approximately 70% yield. A second recrystallization gave (30) in a ≧200:1, and (31) in a 65:1 C20 R:S ratio, both in at least 55% yield. Repeated recrystallization of the mother liquors of (30) added another 10.8% of diastereoisomerically enriched (C20 R:S ratio 100:1) material. With aldehyde (32) we did not pursue recrystallization in the same degree of detail, although it also recrystallizes well from acetone, because a better purification was found at the next step. As a result of the above optimizations, diastereomerically enriched material (20R:S≧200:1) can be obtained in 3-steps and 65% yield (compound (30)) and over 40% yield (compound (31)) respectively.

Aldehydes (30), (31) and (32) are very valuable intermediates for synthesis of pharmaceuticals with the unnatural, 20β configuration. A great deal of chemistry has been developed to elaborate the C22 S-aldehyde position, which is usually obtained by oxidative cleavage of ergosterol, and most of that chemistry could be used on R-aldehydes (30)-(32) (Kutner, A., Perlman, K. L., Sicinsld, R. R., Phelps, M. E., Schnoes, H. K., DeLuca, H. F. Tetrahedron Letters, (1987), 28, 6129-6132). From this literature, it is known that many different nucleophiles can be added to the C22 aldehyde, without any epimerization of C20, and these intermediates can be elaborated to steroidal-5,7-diene precursors of Vitamin D analogues via full elaboration of the C17 side chain by methods known to those skilled in the art.

For example, use of a Wittig reaction or other olefination reagents on 20R,3β-(t-butyldimethylsiloxy)22-homopregna-5,7-dien-22-al (19) will lead to extended steroidal side chains with a C22-C23 double bond, which in turn can be elaborated in many fashions, if so desired. As an illustration, for the purpose of synthesizing Becocalcidiol, reaction of aldehydes (30) and (32) with methylenetriphenylphosphorane leads to 20S,3β-(t-butyldimethylsiloxy)22,23-bishomopregna-5,21-diene (36), and 20S,3β-(t-butyldimethylsiloxy)22,23-bishomopregna-5,7,21-triene (37), which can be selectively catalytically reduced to the key intermediates 20S,3β-(t-butyldimethylsiloxy)22,23-bishomopregna-5,7-diene (38) and 20S,3β-(t-butyldimethylsiloxy)22,23-bishomopregna-5,7-diene (39) respectively. The same sequence on aldehyde (31) produced 20S,3β-(triisopropylsiloxy)22,23-bishomopregna-5,7-diene (16). Use of more complex Wittig reagents, Horner-Wadsworth-Emmons reagents, etc. will lead very conveniently to more elaborate side chains, and some of these are illustrated below.

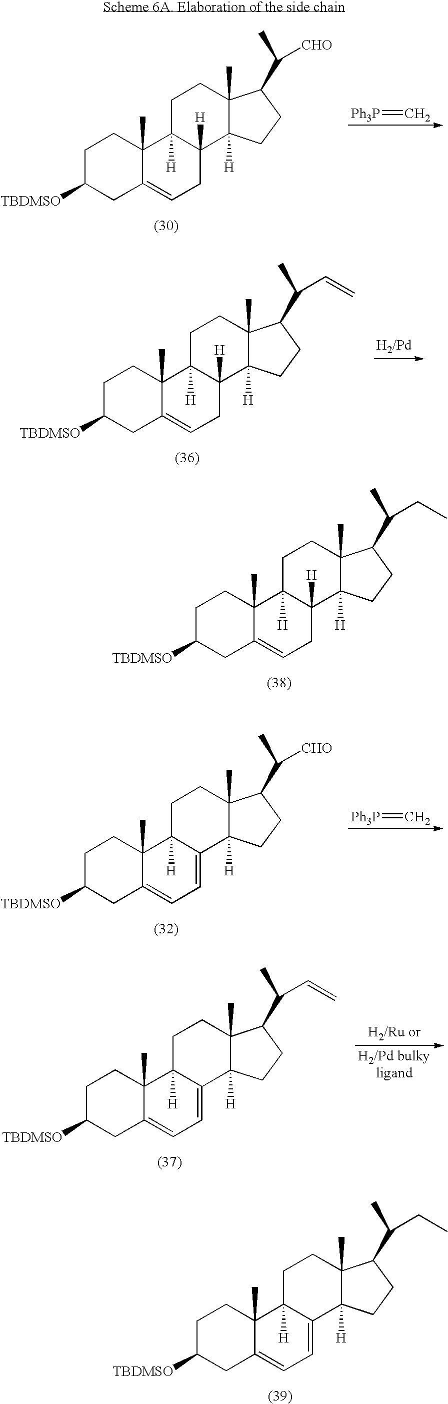

In yet another illustration of the utility of aldehydes (30)-(32) they may be reacted with reagents such as PPh₃/CBr₄, followed by butyl lithium or diethyl 1-lithio-1-diazophosphonate, thereby producing alkyne derivatives (40)-(42). These compounds can be elaborated to a wide variety of 20-epi-steroids, using reactions familiar to one skilled in the art, such as alkylations, electrocyclic, and electrophilic additions on the alkyne to elaborate out many different kinds of side chain.

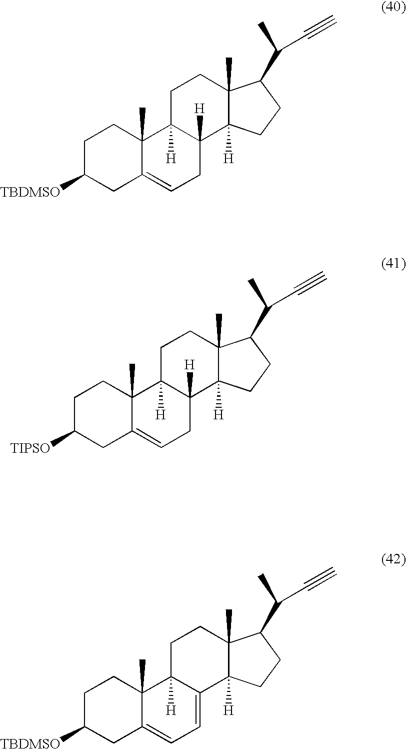

Aldehydes (30)-(32) can be reduced to the corresponding primary [R]-alcohols (43)-(45) by a very wide array of reducing agents (as described in Larock's Modern Synthetic Reactions) with no loss of C20 stereochemical purity. Particularly favored reagents include metal hydride reducing agents such as, but not limited to, DIBAL, NaBH₄ and LiAlH₄. All three [20R]-alcohols are readily distinguished from their [20S]-epimers by thin layer chromatography, and can be obtained essentially diastereomerically pure (20R:S≧200:1) by column chromatography, in 40-60% isolated yield from pregnenolone, or by recrystallization protocols. This means that sufficiently diastereomerically enriched material for drug substances can be obtained in 4-steps and over 40% yield from pregnenolone. These alcohols are also valuable intermediates for the synthesis of pharmaceuticals with a 20S configuration.

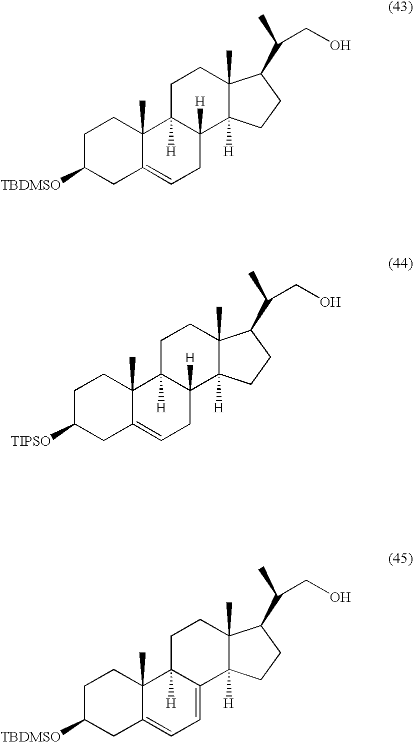

In an especially favorable manifestation of the invention, the epoxide rearrangement and the aldehyde reduction can be combined into a single step, precluding isolation of the aldehyde. As this can be carried out on crude epoxide, it means that the only purification step introduced during the entire side chain synthetic sequence to this point is the chromatography at this step, although the chromatography can be replaced by recrystallization, albeit at some loss of yield. By way of illustration, carrying out such a two step transformation on epoxide (27), alcohol (43) can be obtained in 79.5% yield, which is 74% overall on pregnenolone. 20R,3β-(t-Butyldimethylsiloxy)-22-homopregna-5,7-dien-22-ol (45) can be obtained in very high isomeric purity, by using recrystallized aldehyde, or by recrystallization, or column chromatography of less isomerically pure aldehyde. Ethyl acetate has been found to be a good solvent for this recrystallization, and two recrystallizations can improve the DE of alcohol (45) to >98%.

Treatment of alcohols (44) and (45) with tosyl chloride in dichloromethane containing 4-(N,N-dimethylamino)pyridine and triethylamine gives the corresponding tosylates (46) and (47) in over 80% yield, after recrystallization from acetonitrile, which improves the diastereoisomer excess usefully, if the alcohol was of DE≦98%. Similarly alcohol (44) was converted into the corresponding mesylate ester, and all three alcohols could be converted to a wide variety of sulfonate esters, which can be used as electrophiles in nucleophilic displacement reactions and coupling reactions, as is known to one skilled in the art.

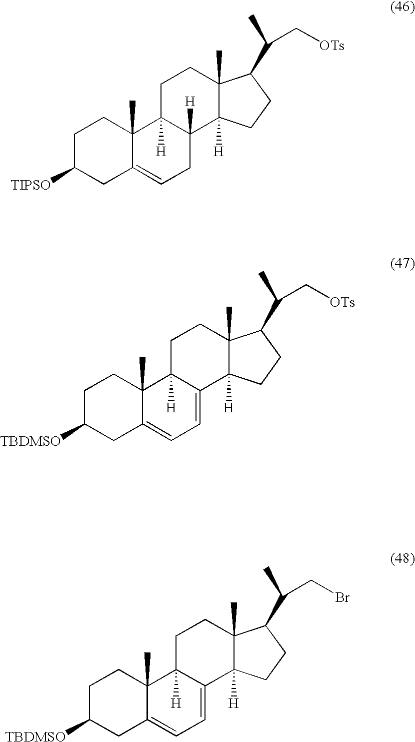

Another useful transformation of alcohols (43)-(45) is conversion of the alcohol into a halide, preferably bromide or iodide, for example by use of appropriate phosphorus halide derivatives, or Ph₃P/CX₄, or other techniques disclosed in “Comprehensive Organic Transformations 2^{nd} Edition” by R. C. Larock followed by displacement of the halide by an appropriate nucleophile. The conversion of 20R,3β-(t-butyldimethylsiloxy)22-homopregna-5,7-dien-22-ol (45) into 20R,3β-(t-butyldimethylsiloxy)-22-bromo-22-homopregna-5,7-diene (48) was carried out in 88% yield using CBr₄/PPh₃ in presence of collidine as a base. This transformation is especially advantageous since these halides can readily be turned into the corresponding organometallic reagents, such as lithio, magnesio, zincato and cuprato derivatives, all of which can then be reacted with appropriate electrophiles, such as alkyl halides/sulfonates, Michael acceptors and epoxides, to elaborate the steroidal side chains efficiently, using techniques known to one skilled in the art.

### Specific Uses of Intermediates Described Above.

### 1. Synthesis of (20S)-1 α-hydroxy-2-methylene-19-norbishomopregnacalciferol (Becocalcidiol)

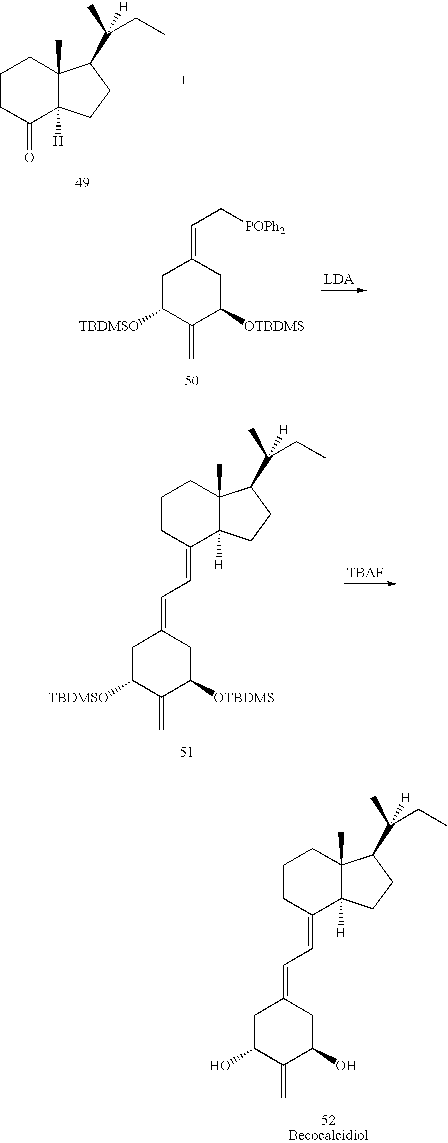

(1R,3αR,7αR)-7-Methyl-1-([1S]methylprop-1-yl)octahydroinden-4-one, ((1R,6R,7R)-6-methyl-7-([1S]methylprop-1-yl)bicycle[4.3.0]nonan-2-one) (49), is coupled with the phosphine oxide (50) to form the protected Vitamin D analogue (51), which can be readily desilylated to synthesize (20S)-1α-hydroxy-2-methylene-19-norbishomopregnacalciferol, (52). Compound (52) is described generically in U.S. Pat. No. 5,936,133, and in U.S. Pat. No. 6,627,622. Its crystalline form is disclosed in U.S. Pat. No. 6,835,723. Compound (52) and its utilities are claimed in U.S. Pat. No. 6,887,860, where the synthesis is stated to involve a classical Lythgoe condensation of the Windhaus-Grundmann ketone analogue (49) with the allylic phosphine oxide (50), to give the bis-silylated product (51), which is deprotected by fluoride ion-induced hydrolysis to give (52). As compound (52) has valuable Vitamin D agonistic effects, whilst having little hypercalcemic effect it is useful as a potential medication for a variety of conditions as disclosed in US 20040033998 A1. As a key intermediate in the synthesis of diene (52), ketone (49) therefore has utility as a synthetic intermediate, and methods of making (49) which would allow it to be produced more readily and/or at lower cost than at current methodologies, which are not particularly efficient, would be advantageous. The current invention can be used to produce ketone (49) much more cheaply, and in considerably better yield than the described route from ergosterol. (DeLuca, H. F.; et al. U.S. Pat. No. 6,835,723).

Compound (49) presents several synthetic problems. It is chiral, and a trans bicyclo[4.3.0]nonan-2-one. It has a quaternary center and a cis-6,7-dialkyl substitution pattern, and the steroidal side chain has the unnatural [S]-configuration at C20. By starting with a naturally occurring steroid one can readily solve the problems of chirality, the quaternary center and the trans-bicyclononanone structure. However, one must be able to ensure that the steroidal A and B rings are efficiently removed, whilst leaving only the C2 (C8 steroidal) position functionalized, and one must also ensure the correct stereochemistry at C17 and C20, and that the C14 stereochemistry is retained. There are two known processes for ensuring that the AB ring is cleaved, whilst leaving a functionality at C8, which can be used to elaborate the desired Vitamin D analogues. One must either start with a B-ring 5,7-diene or introduce it, and then photochemically open the diene to a triene followed by a 1,7-hydride shift, exactly as occurs in the conversion of preVitamin D to Vitamin D. The 7,8-alkene is then cleaved oxidatively to introduce the 8-ketone. Compound (39), like cholesterol, has no functional groups in its C17 side chains, and can therefore be photolysed followed by a 1,7-hydride shift, under the conditions described for the 7-dehydrocholesterol to Vitamin D₃ conversion (M. Okabe. Organic Syntheses, 76, 275, (1999)) to turn it into triene (53), which can then be ozonized to ketone (49). An alternative, which involves the direct ozonolysis of a steroidal monoene, such as (16) or (38) followed by photochemical removal of the entire A-ring, will be discussed later.

Both tosylates (46) and (47) couple very efficiently with MeMgBr in the presence of Li₂CuCl₄ catalyst, to give the key intermediates 20S,3β-(triisopropylsiloxy)-22,23-bishomopregn-5-ene (16) and 20S,3β-(t-butyldimethylsiloxy)-22,23-bishomopregna-5,7-diene (39) in 90-100% crude yields and high purity. Both of these compounds can be purified further by chromatography or via crystallization. Conversion of monoene (16) into the corresponding 5,7-diene (15) via the “Confalone” sulfoxide route was described above in Scheme 3.

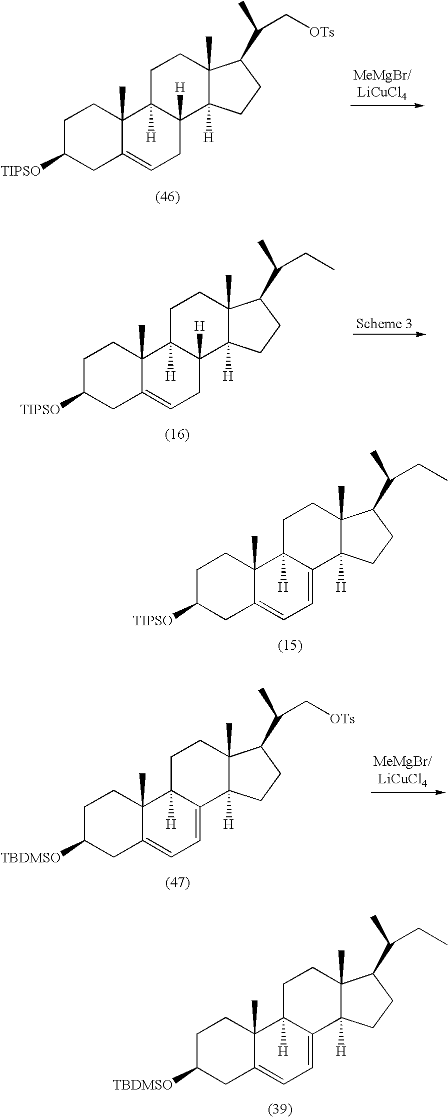

The dienes (15) and (39) are chemically very close analogues of 7-dehydrocholesterol, and of ergosterol, and can be photochemically ring opened to the Vitamin D triene analogues under similar conditions to those used in commercial Vitamin D syntheses. (See M. Okabe. Organic Syntheses, 76, 275, (1999). Steroidal 5,7-diene (39) has been photolysed as described by Okabe with a Hanovia mercury lamp, to give a mixture of the pre-Vitamin D analogue (54) and the tachysterol analogue (55). Reirradiation with longer wavelength radiation (uranium filter) converts most of the unwanted tachy-isomer (55) to the pre-Vitamin D analogue (54), which is then thermally equilibrated to a mixture of triene (54) and Vitamin D triene analogue (53), favoring the latter by about a 10:1 ratio. Triene (53) can be ozonized to form the key ketone intermediate (49), a Windhaus-Grundmann ketone, which is a well known reaction in Vitamin D chemistry. Because of the possible lability of the trans ring junction in ketone (49), it was not directly isolated, but was reduced to the known trans-octahydroindanol (56) in situ. Alcohol (56) was obtained pure, in overall 36% yield from diene (39) in this four step process in up to a gram scale. It is anticipated that this yield can be improved by using better photolysis apparatus, such as recirculating photolysis apparatus, and falling film apparatus. Alcohol (56) can be oxidized to ketone (49) in 99% yield with pyridinium dichromate, as described in the literature. (DeLuca, H. F.; et al. U.S. Pat. No. 6,835,723 (2004)).

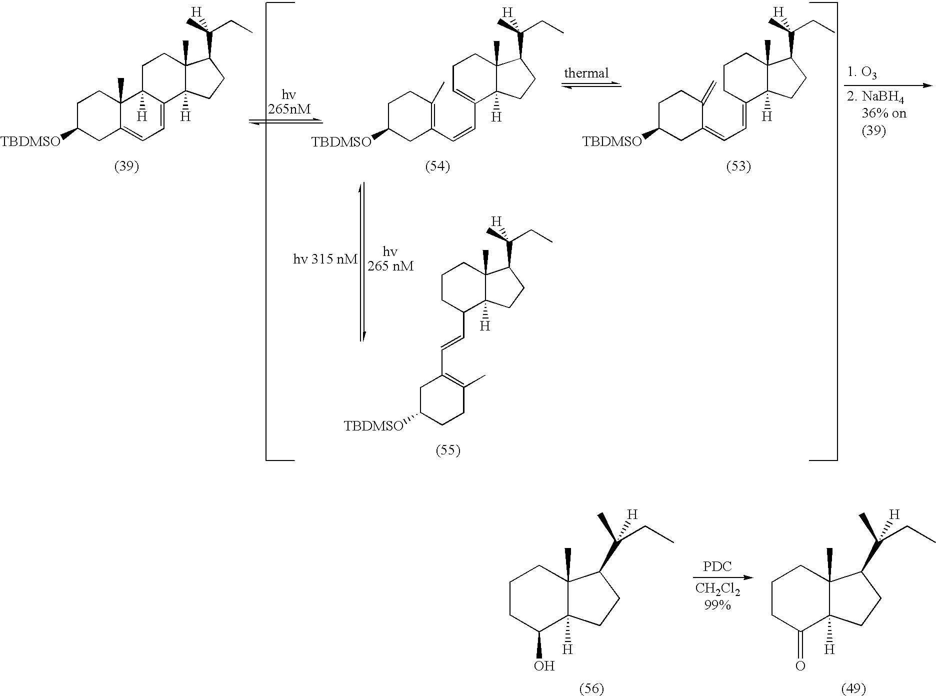

Thus overall, as shown in Scheme 7, this chemistry represents a 16 reaction synthesis of ketone (49) from pregnenolone (1). Two of the steps can be functionally simplified by being carried out in situ, and both photolyses, the thermal isomerization and the ozonolysis/reduction are carried out without a purification, and only an evaporation down and reconstitution of the reaction solution, leading to an 11 “pot” conversion. Each of the isolated intermediates is a crystalline solid, and can be recrystallized if required.

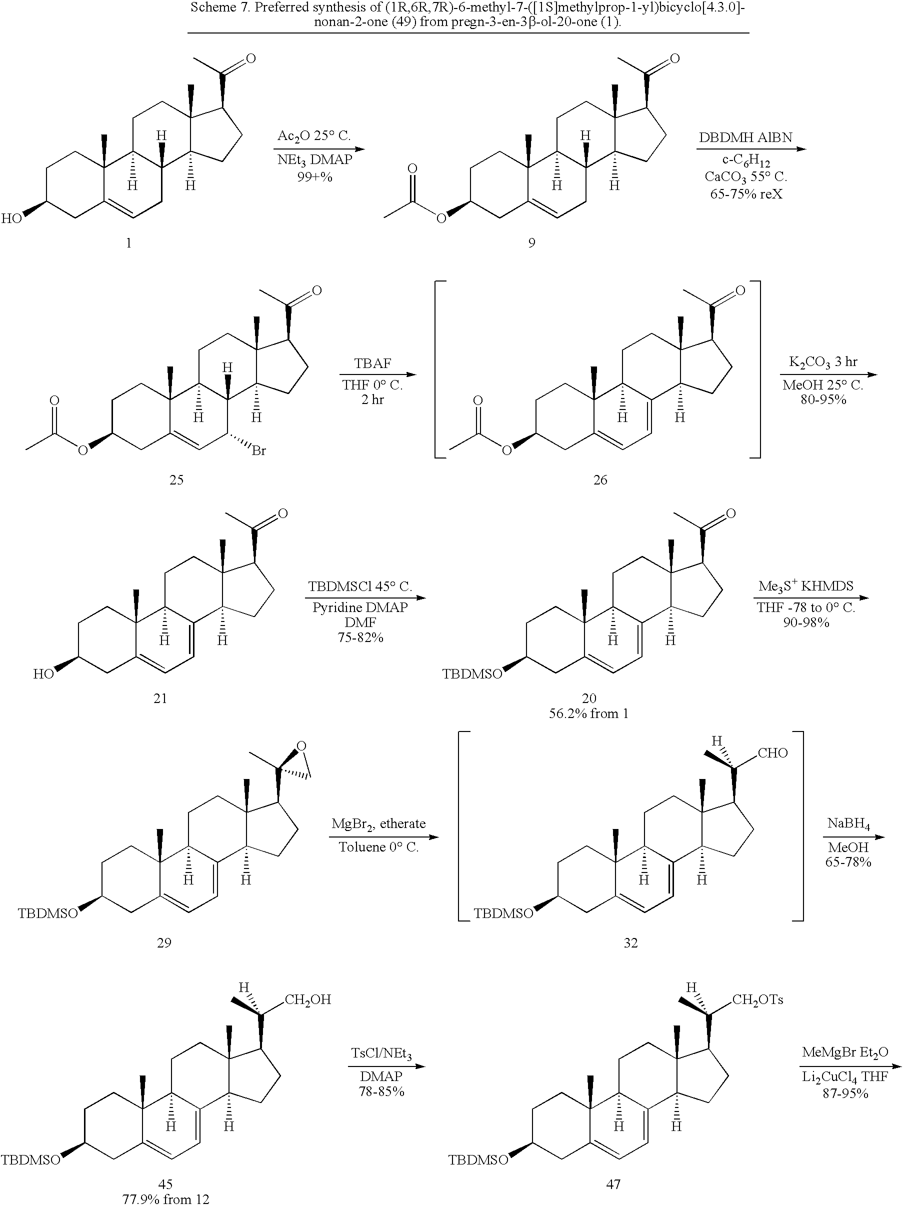

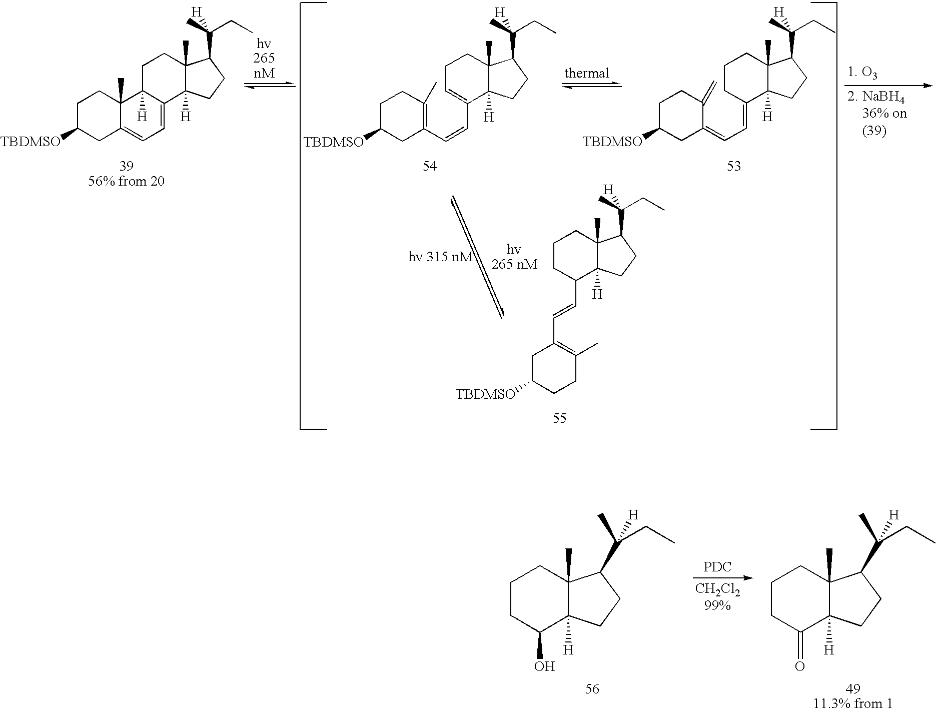

Ketone (49) was treated with the lithium anion of phosphine oxide (50), as described in the literature, and underwent Lythgoe coupling to give the Vitamin D analogue (51) in 79.7% yield. TBAF deprotection, and crystallization gave Becocalcidiol (52) in 85.1% yield, as described in the literature. Thus, this process synthesized Becocalcidiol (52) in overall 7.6% yield from pregnenolone (1).

### 2. Synthesis of (20S)-1α,25-dihydroxy-2-methylene-19-norcholecalciferol and their 26,27-bishomo and 26,27-cyclobishomo homologues

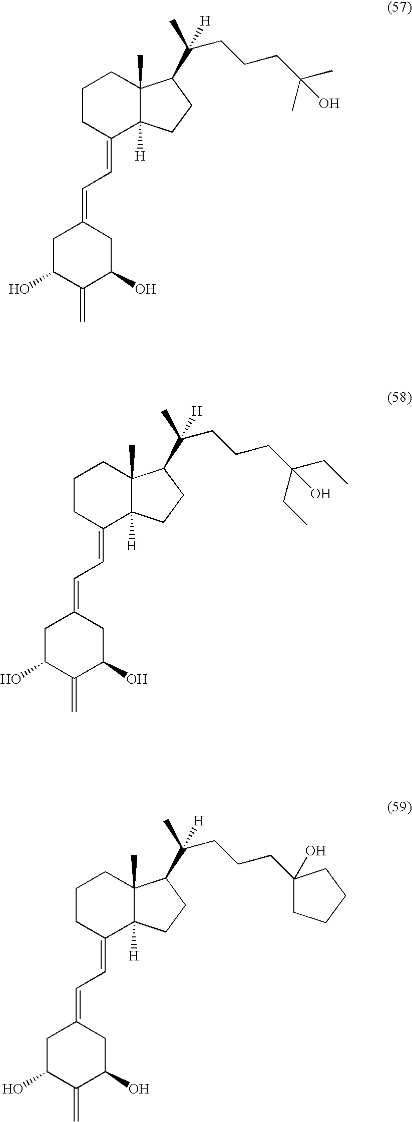

Compounds such as (57), (58) and (59) are described as having interesting calcaemic properties, (DeLuca and Sicinsld; U.S. Pat. Nos. 6,392,071 issued May 22, 2002, 6,544,969, issued May 8, 2003, 6,537,981 issued Mar. 25, 2003. Shevde, N. K et al. Proc. Natl. Acad. Sci. USA, 99, 13487-13491, (2002)) and only differ from compound (52) in the nature of their C17 side chain. Thus these compounds can be made from intermediate (47) simply by coupling the appropriate alkyl groups to it. One way this can be done readily is by coupling (47) or (48) with O-protected Grignard reagents, exemplified by the TBDMS derivatives (60), (61) and (62), but which can use other alcohol protecting groups known to one skilled in the art (Greene and Wuts, Protective Groups in Organic Synthesis 3^{rd} Edn.), using copper reagents as described immediately above, to give the 20-epi-7-dehydrocholesterol analogues (63)-(65). Clearly (63)-(65) be also made from compounds such as (32), via a Wittig reaction, followed by reduction at an appropriate later stage, and various other metal-induced coupling reactions obvious to one skilled in the art. Carrying these compounds through the photolysis-ozonolysis sequence will give the CD-ring ketones (66)-(68), which can then be Lythgoe coupled (or Julia sulfone coupled) with phosphine oxide (50) to produce Vitamin D analogues (57)-(59) after desilylation.

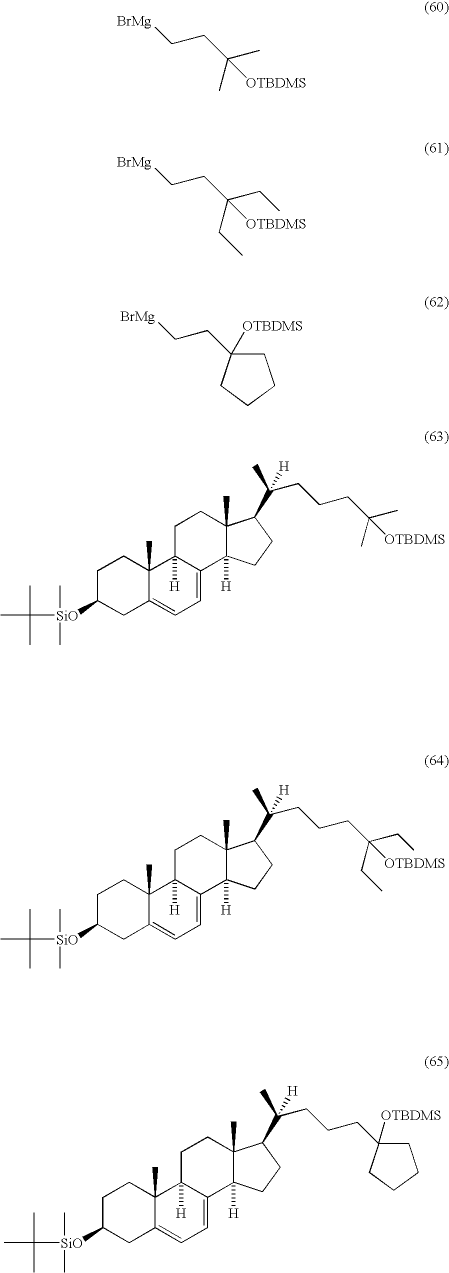

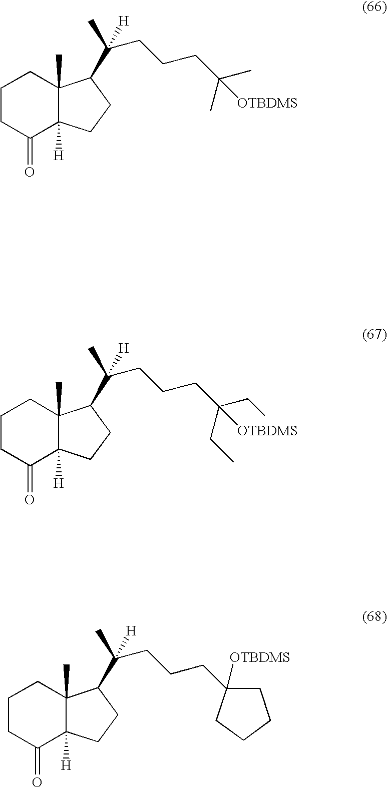

Another method by which these side chains may be attached to the [20R],C22-homologated pregnenols, is to convert alcohols such as (45), sulfonates, exemplified by (47) and halides exemplified by (48) to the corresponding sulfides, exemplified by aryl sulfide (69). This can be done via a Mitsunobu reaction on (45), or simple nucleophilic displacement of the leaving groups of (47) and (48) with a thiolate anion. Oxidation of the sulfide to the sulfone (70) may be difficult in the presence of the diene, but (45), (47) and (48) can be converted directly to the sulfone by use of an appropriate sulfinate nucleophile (Schrotter, E., Schonecker, B., Hauschild, U. Droescher, P, Schick, H. Synthesis, 193-5 (1990).). Generation of an anion at the C22 position can be carried out with alkyl lithium or lithium amide bases, and these in turn can be alkylated as described in the literature (Schrotter, E., Schonecker, B., Hauschild, U. Droescher, P, Schick, H. Synthesis, 193-5 (1990)), to produce compounds such as (71), which can be desulfonated to produce the corresponding epi-cholesterol derivatives, in this case (63).

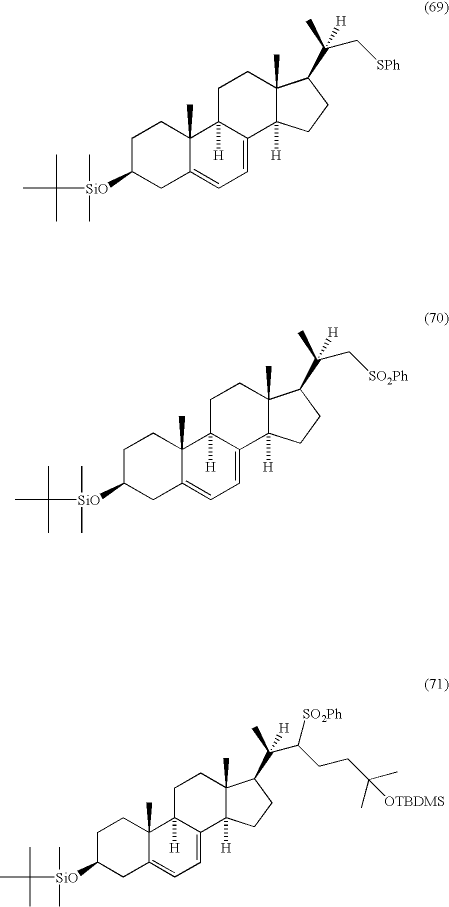

### Synthesis of 20-epi Vitamin D₃, 25-Hydroxy-20-epi Vitamin D₃ and 1,25-Dihydroxy-20-epi Vitamin D₃

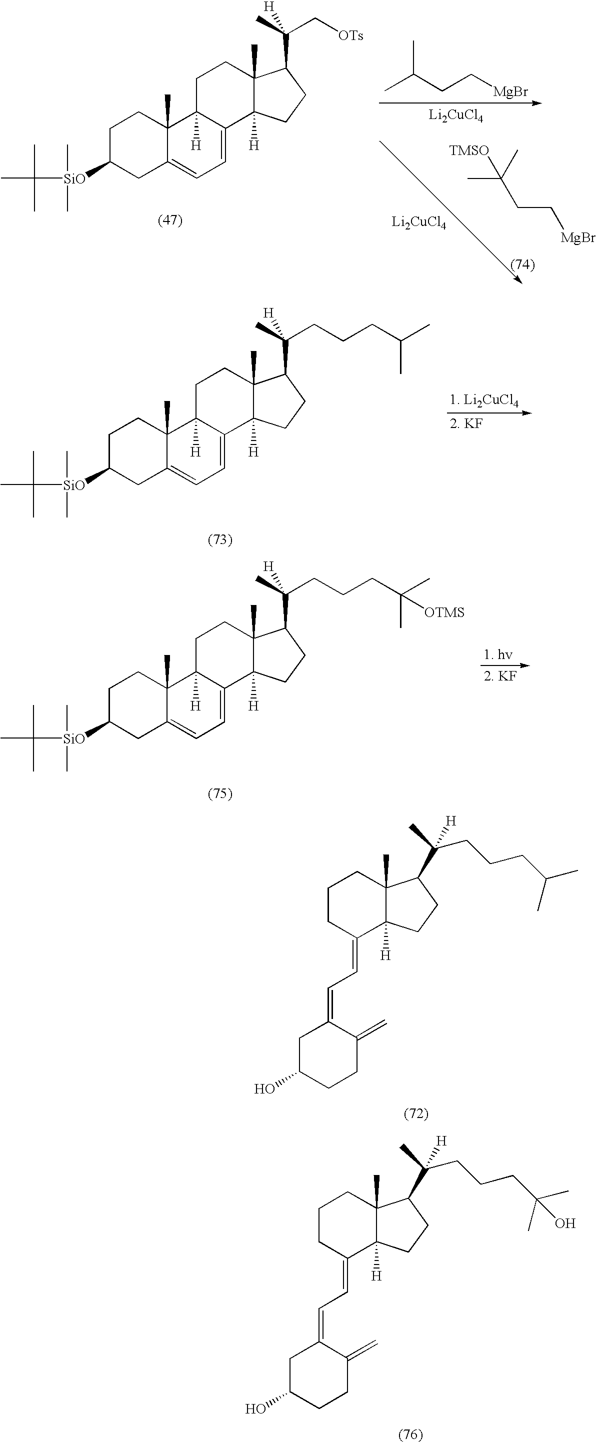

One way 20-epi Vitamin D (72) can be readily prepared is by coupling tosylate (47) with isopentyl magnesium bromide to give 20-epicholesta-5,7-diene (73), followed by photolysis and deprotection. Clearly the aldehyde (32) can be converted to (72) by several other methods, obvious to one skilled in the art. Similarly, coupling of (47) with the corresponding 3-silyl ethers, such as (74) to form the protected cholestadiendiol (75), followed by photolysis and deprotection will lead to 20-epi-25-hydroxy Vitamin D (76).

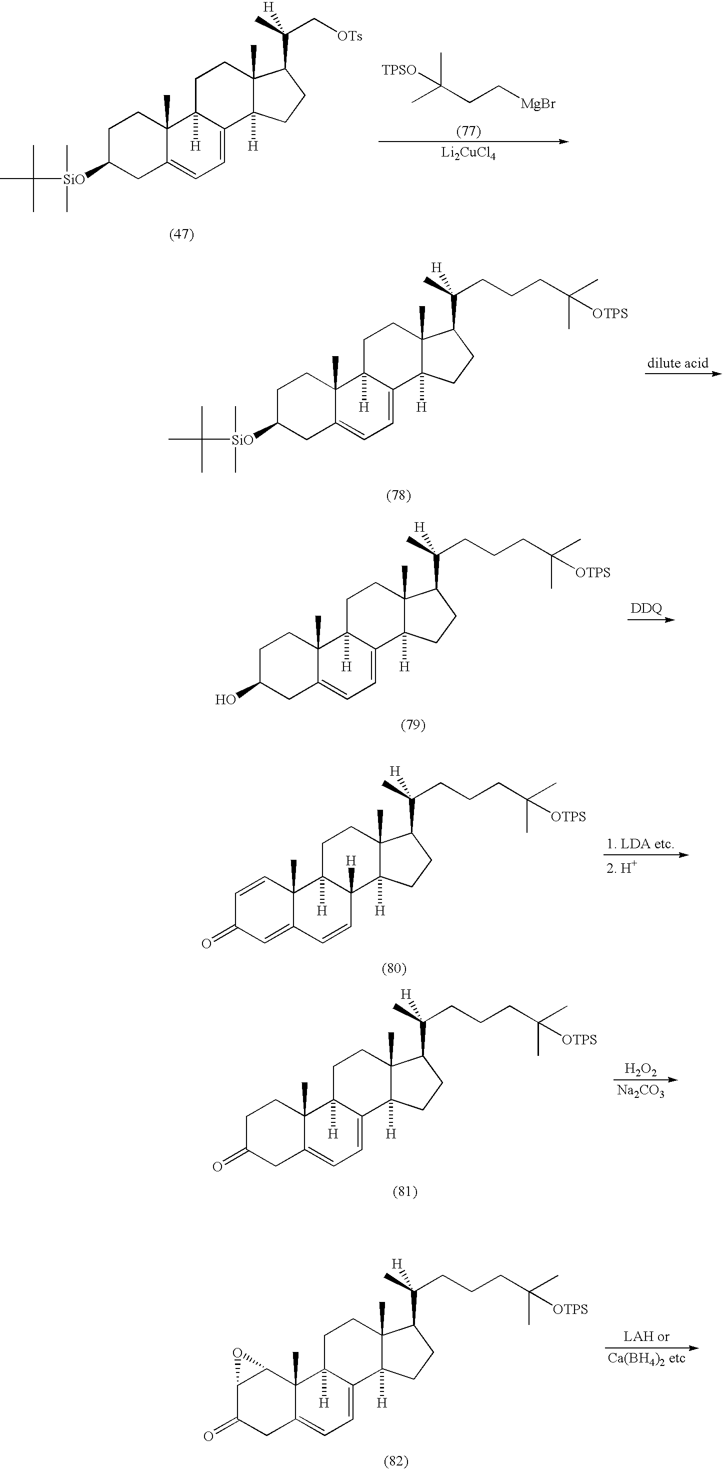

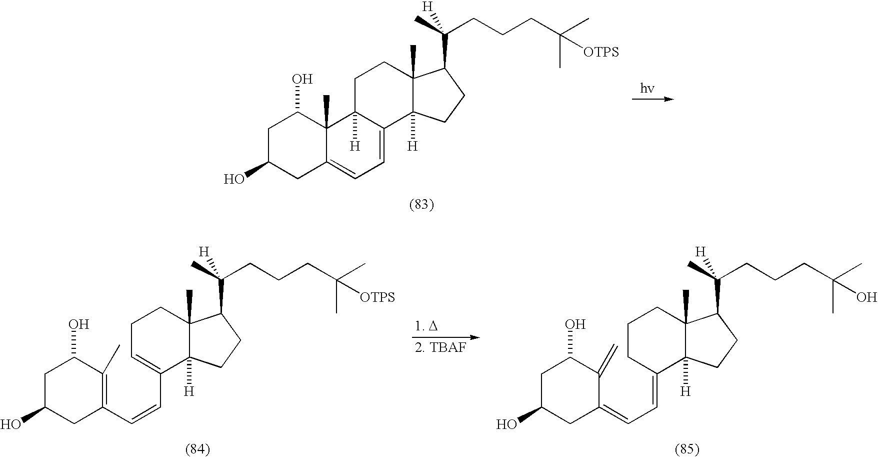

Because of the economic importance of 1α-Vitamin D derivatives, the chemistry of C1-hydroxylation of cholesterol and its derivatives has been well worked out. (Zhu, G.-D., Okamura, W. H. Chem. Rev. 95, 1877-1952, and references therein). Reaction of tosylate (47) with an appropriately orthogonally protected 4-hydroxy-4-methylbut-1-yl Grignard reagent exemplified by TPS (triphenylsilyl, but TBDPS, t-butyldiphenylsilyl may work as well) ether (77) will give the key intermediate (78). Selective deprotection of the 3-silyl ether under acidic conditions will give alcohol (79) which on oxidation with chloranil or DDQ leads to oxidation to the trienic ketone (80). Treatment of (80) with a strong base leads to abstraction of the H8 proton, and formation of a trienolate, which upon kinetic reprotonation forms the deconjugated 1,5,7-trien-4-one (81) (Guest, D. W. and Williams D. H. J. Chem. Soc. Perkin 1, (1979), 1695). Treatment of this compound, or appropriate derivatives of it, with mildly basic hydrogen peroxide forms the 1α,2α-epoxide (82), which upon reduction with hydride reducing agents such as LAH or Ca(BH₄)₂, will open the epoxide trans-diaxially, and reduce the ketone to the equatorial alcohol to give the 1α,3β-diol (83). Alternatively, epoxidation of (80) as described above, followed by a Li/NH₃ reduction will give a-1α,3 β-6-cholestene derivative, which can be brominated and doubly dehydrobrominated to give (83) (Dreeman, D., Acher, A., Mazur, Y. Tet. Letters, 16, 261-4 (1975)). Photolysis under the usual Vitamin D wavelength restraints with appropriate sensitizers at low temperature gives the corresponding pre-Vitamin D₃ derivative (84). Thermal 1,7-hydride shift gives the protected Vitamin D₃ analogue, which can be deprotected with fluoride ion to form 20-epi-1α,25-dihydroxy Vitamin D (85). Alternatively, the TPS (TBDPS) group may be removed before the photolysis. Or the 1,3-dihydroxy groups may need to be appropriately protected before the photolysis, and deprotected after the photolysis, along with the TPS (TBDPS) group. Other protecting group strategies could be used in the side chain, as loss of the tertiary alcohol protecting group prior to the DDQ oxidation should not be problematic, and a wide variety of protecting groups could be reintroduced to the tertiary alcohol immediately after DDQ oxidation.

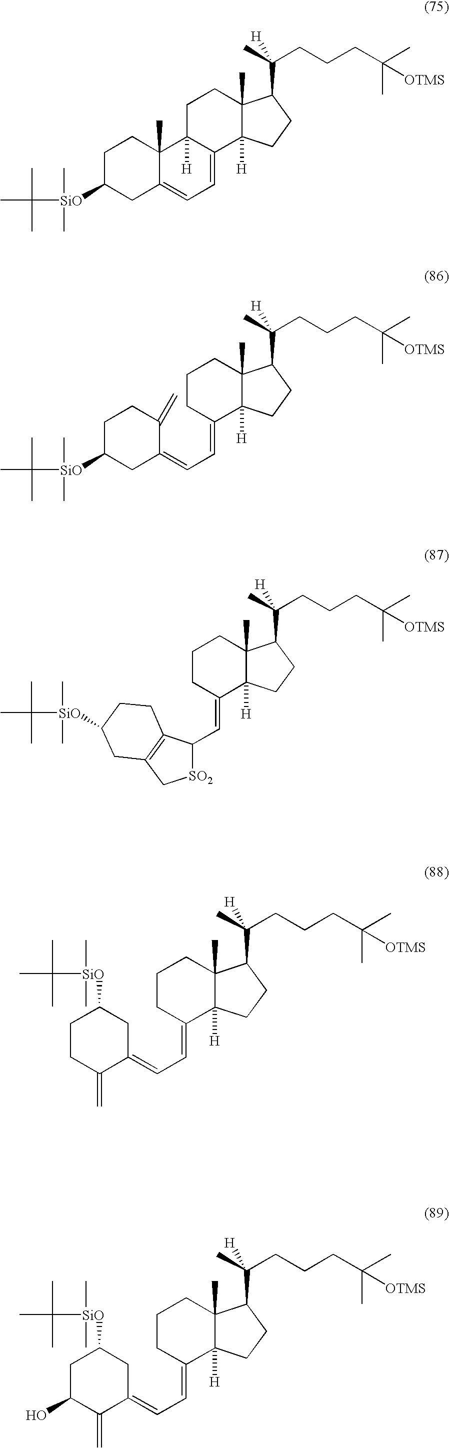

An alternative preparation of (85) involves photolysing the protected diol (75) and thermally isomerizing it to triene (86). (R. Hesse, U.S. Pat. No. 4,772,433. Andrews, D. R. et al. J. Org. Chem., 51, 4819 (1986). DeLuca, H. F. et al. U.S. Pat. No. 4,265,822) Dissolving triene (86) in liquid sulfur dioxide will produce the sulfolene (87), which on thermal cheleotropic elimination gives the isomerized triene (88). This can be allylically oxidized with SeO₂ or similar reagent described in “Comprehensive Organic Transformations 2^{nd} Edition” by R. C. Larock to give the alcohol (89). Photoisomerization of the 5,6-double bond and deprotection will give (85).

### 4. Synthesis of 20-epi Calcipotriene (90), 20-epi Falecalcitriol (91) and 20-epi Seocalcitol (92)

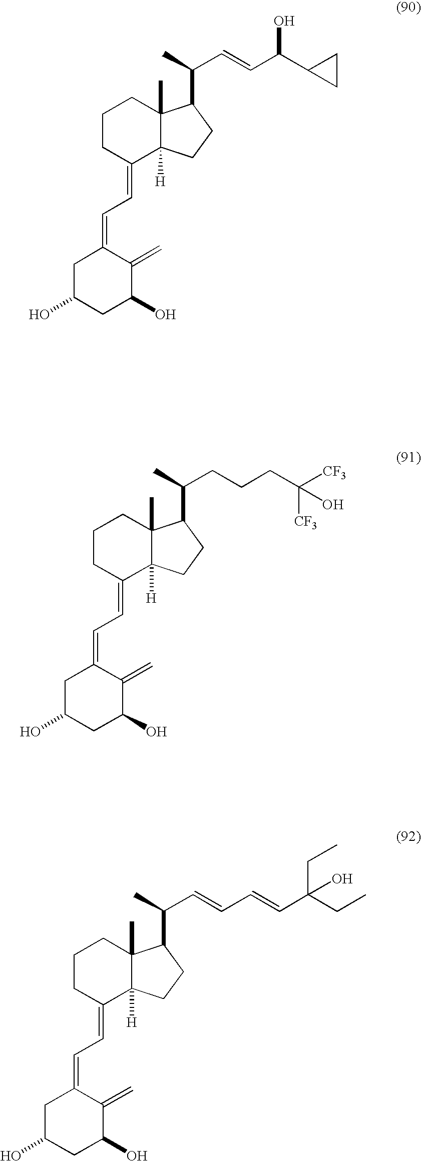

The chemistry described above can be used to efficiently produce the C20 epimers of several important Vitamin D derivatives. The above three compounds are 1α-hydroxy Vitamin D derivatives, and can be readily obtained in protected form from either compound (32) or (47/88), or their TIPS-protected analogues. Thus, for example to prepare (90), the sequence in Scheme 8 can be used.

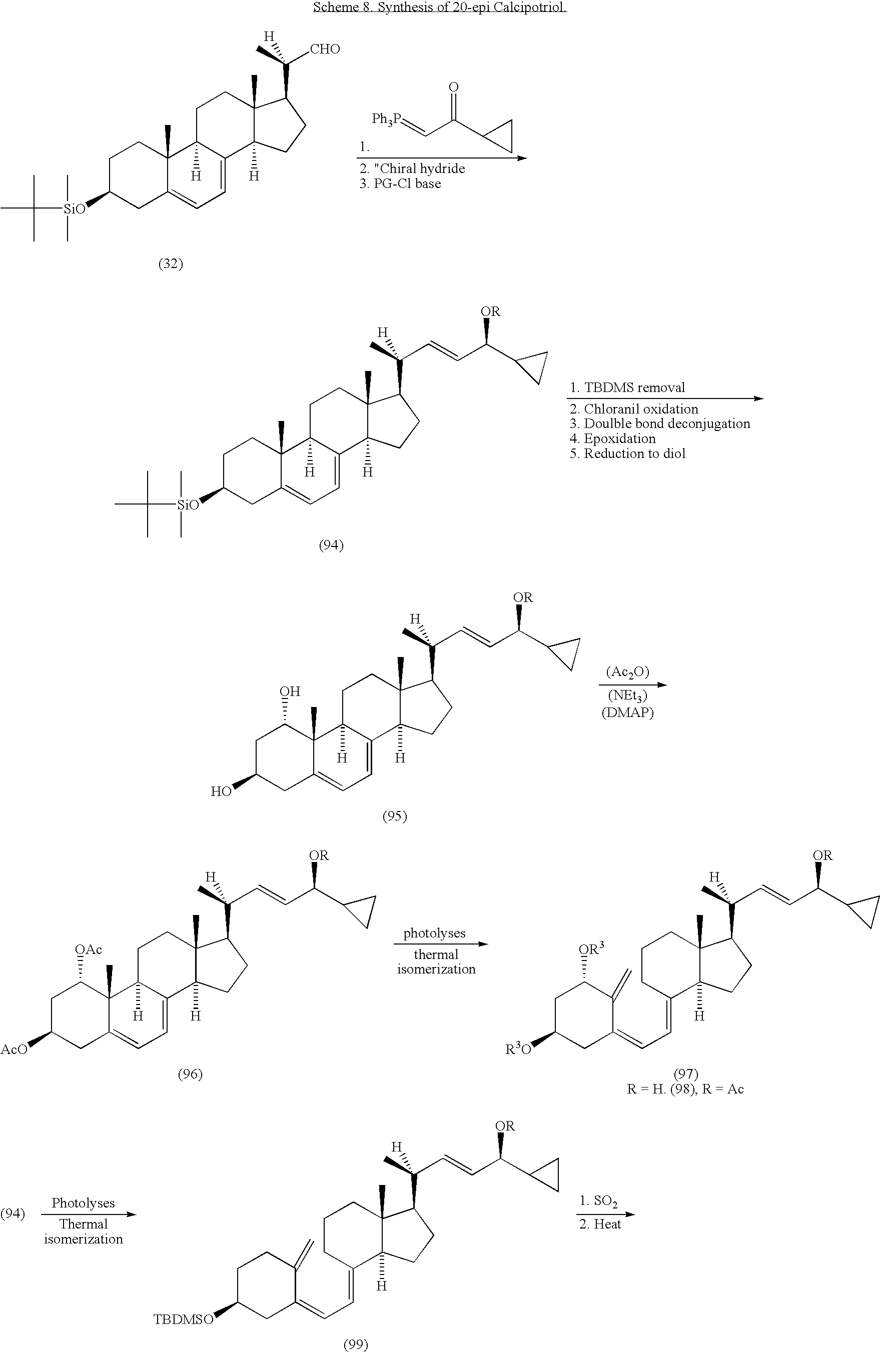

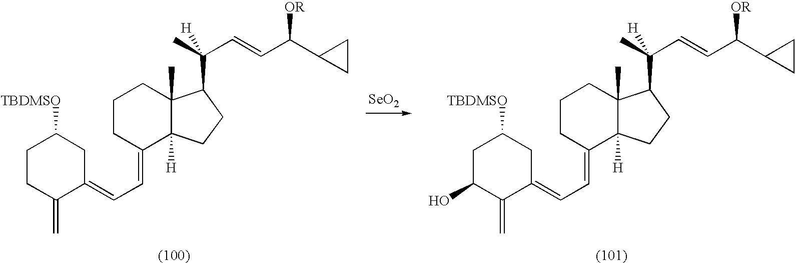

Treatment of aldehyde (32) with stabilized ylide (93) or other appropriate olefinating agent, followed by a chiral ketone reduction, (see “Handbook of Reagents for Organic Synthesis; Chiral Reagents for Asymmetric Synthesis. Ed L. A. Paquette) and PG-Cl=MEM or TBDPS chloride will give the steroidal 5,7-diene precursor (94, R=MEM or TBDPS). This can be hydroxylated by the DDQ/cloranil route, described above, to give the protected steroid (95, R=MEM or TBDPS) or, for example, the diacetate (96, R=MEM or TBDPS) if required. Photolysis/isomerization of this compound gives the protected precursors (97, R=MEM or TBDPS) or (98, R=MEM or TBDPS), which can be deprotected to (90) by a variety of methods familiar to one skilled in the art. Alternatively, photolysis/isomerization of (94, R=MEM or TBDPS) to give 5Z-Vitamin D analogue (99, R=MEM or TBDPS) can be followed by the two step 5,6-isomerization to give the 5E-triene (100, R=MEM or TBDPS), which can be allylically hydroxylated to triene (101, R=MEM or TBDPS), followed by long wavelength reisomerization to the 5Z-triene and deprotection to (90). (R. Hesse, U.S. Pat. No. 4,772,433. Andrews, D. R. et al. J. Org. Chem., 51, 4819 (1986). DeLuca, H. F. et al. U.S. Pat. No. 4,265,822)

A similar route for making 20-epi Falecalcitriol is shown in Scheme 9.

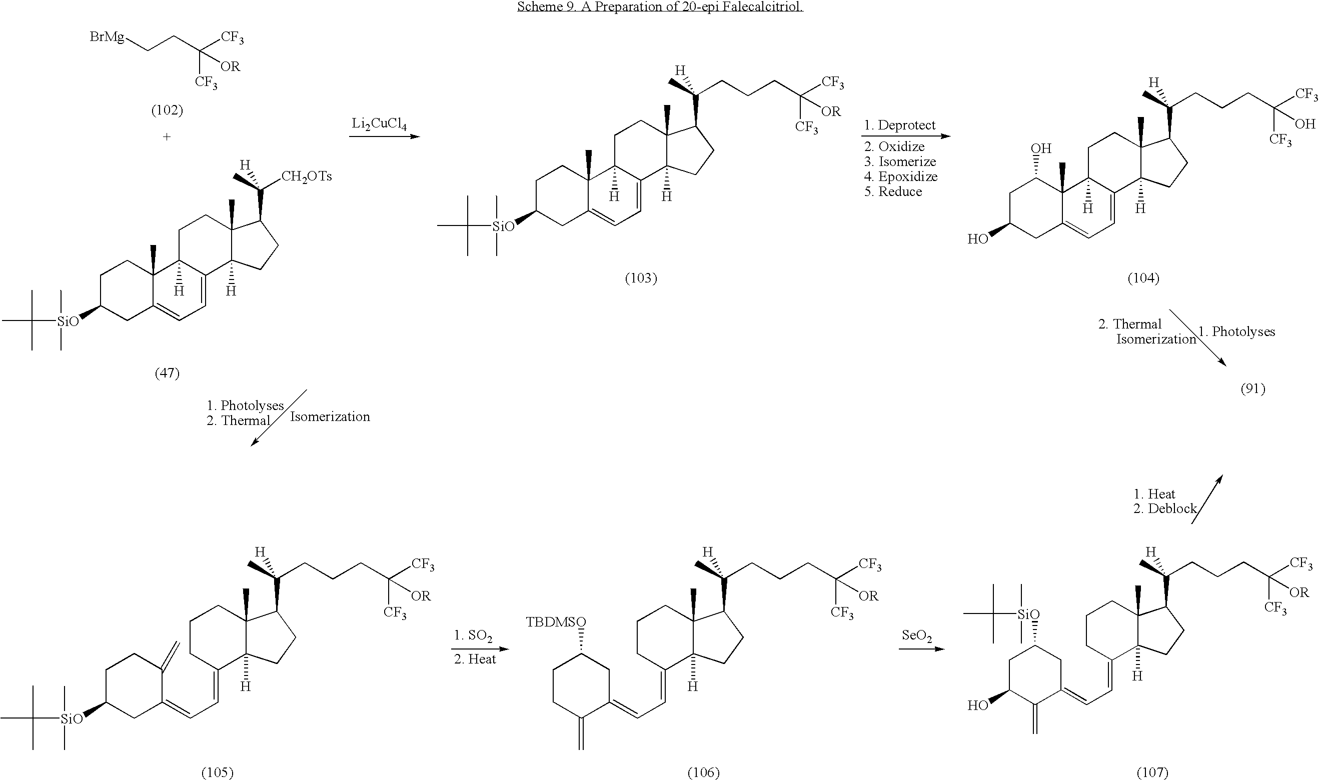

Copper-catalysed reaction of tosylate (47) with a silyl-protected hexafluorinated Grignard reagent (102) will give the steroidal diene (103), which can be converted, either to the 1α-hydroxylated steroid (104) or a 1,3-diprotected analogue, as discussed in Specific Use 3 above. If R is TMS in compounds (102) and (103), it will be removed along with TBDMS from (103), and replaced if needed at the trienone stage by a different protecting group, in which case R will not necessarily be TMS, although it may be most convenient to simply retrimethylsilate one of these intermediates. Compound (104), or its appropriately 1,3-diprotected analogue can then photolysed/isomerized and appropriately deprotected to give (91), or can be photolysed/isomerized directly to triene (105), which in turn can be 5Z to 5E-isomerized via sulfur dioxide cycloaddition-elimination to give (106), which can be allylically hydroxylated to (107), and then 5E to 5Z isomerized by long wavelength photolysis and deprotected, to give (91).

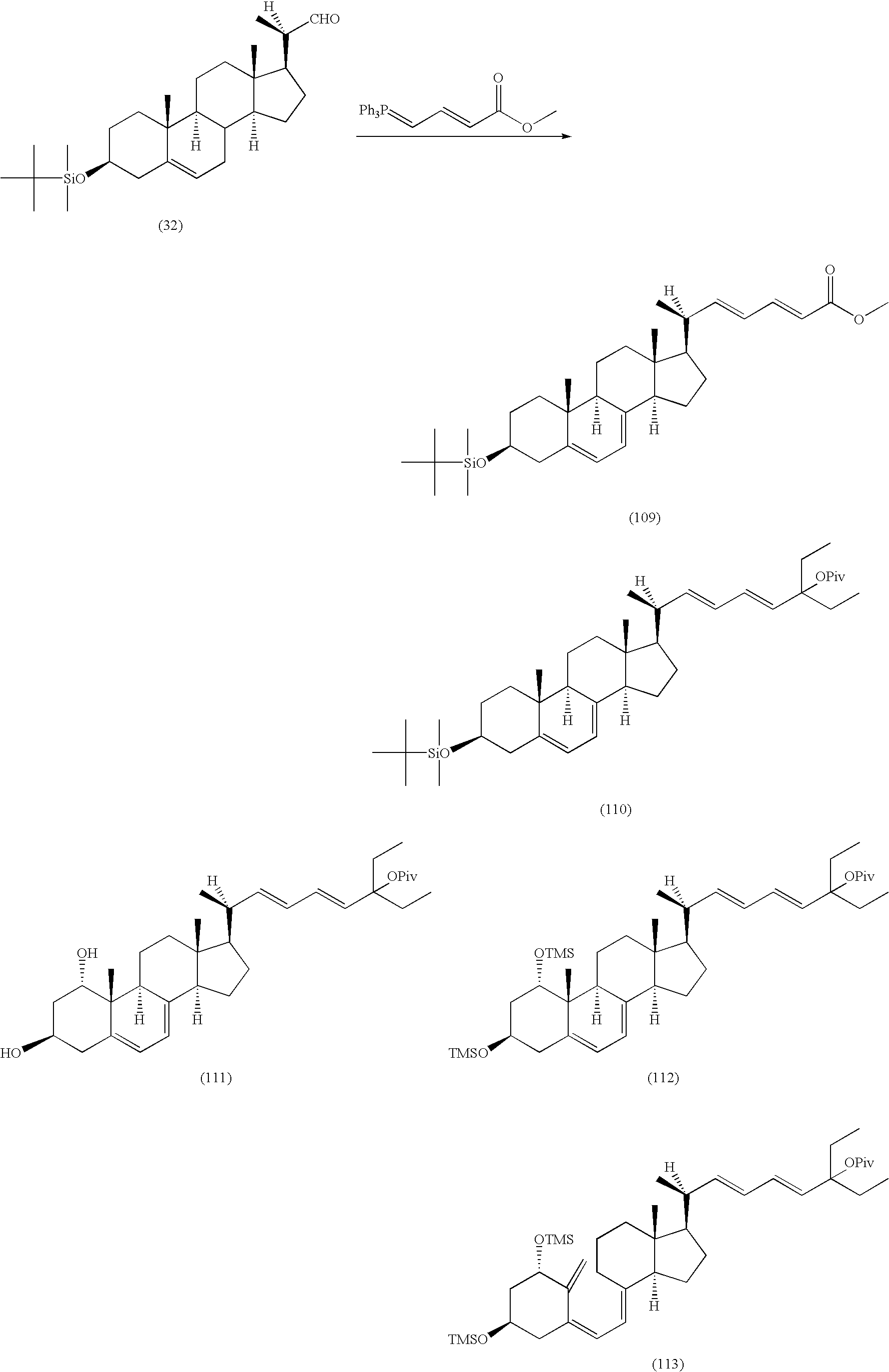

A preparation of (92) can be carried out by analogy with the preparation of (90) from aldehyde (19) as described above. Reaction of (32) with the conjugated stable ylide (108) will give the E,E-dienone (109), which can be reacted with two equivalents of ethyl lithium, and pivaloyl chloride/DMAP to produce key intermediate (110). Desilylation of this, followed by the same DDQ-deconjugation-oxidation-reduction sequence as described previously will give the desired dienediol (111). This can be protected as the bis-silyl (exemplified here by TMS) ether (112), and photolysed/isomerized to 5Z,7E-Δ_{10-19,5-6,7-8} triene (113), which can be deprotected to (92). If the pivaloyl group is lost during introduction of the 1-hydroxy group, the 1,3,25-triol corresponding to (111) can simply be trisilylated to give the 25-TMS analogues of (112) and (113).

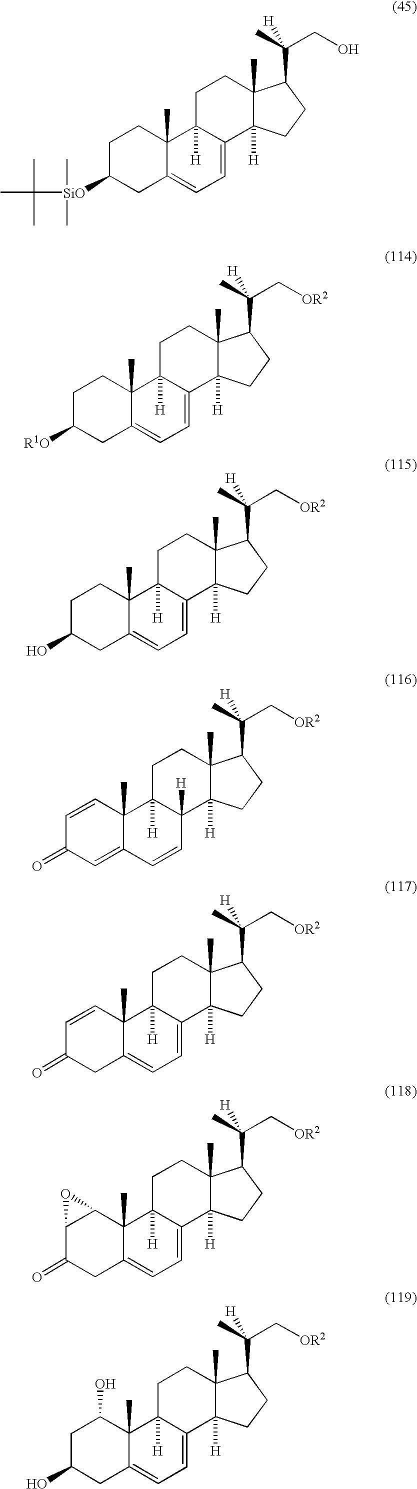

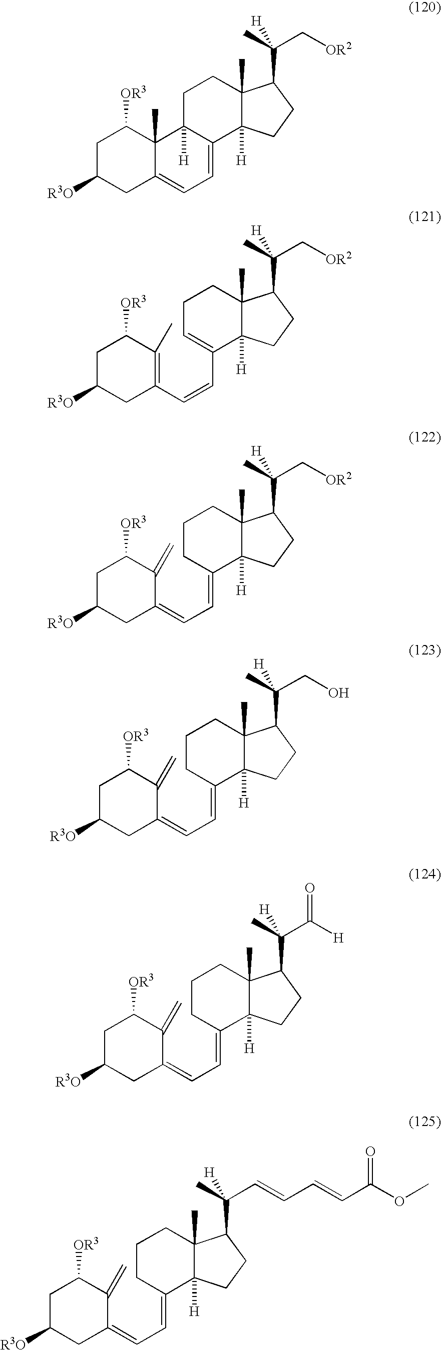

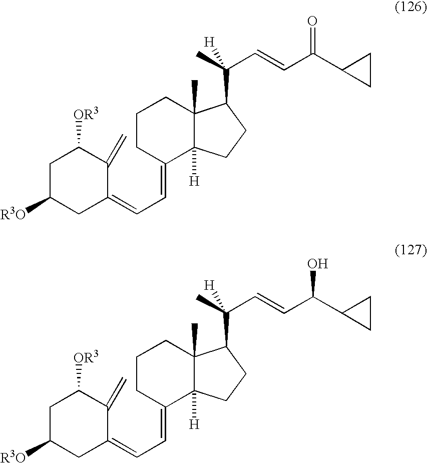

An alternative preparation of 20-epi compounds with a 22,E-double bond is illustrated above. Alcohol (45) can be protected, for example by treatment with pivaloyl chloride or TPS chloride to form (114, R¹=TBDMS, R²=pivaloyl or TPS) and then desilylated to (115, R²=pivaloyl or TPS). Another illustrative example would be to desilylate (45) to diol (114, R¹=R²═H), and then exploit the exceptionally low reactivity of the 3-hydroxy towards TIPS chloride, by selectively silylating the 22-hydroxy to give (115, R²=TIPS). The DDQ oxidation could then be carried out to give trienone (116, R²=Piv, TPS or TIPS) followed by base induced deconjugation to trienone (117, R²=Piv, TPS or TIPS). Peroxide induced oxidation will give epoxide (118, R²=Piv, TPS or TIPS), and appropriate hydride reduction will give diol (119, R²=Piv, TPS or TIPS). Diol (119) can now be orthogonally 1,3-protected, for example if R²=TPS or TIPS, R³═Ac, and if R²=Piv, R³=TMS or TBDMS, with this pattern holding through the protected 5,7-diene (120), the initial photolysis product (121), and the thermally isomerized triene (122). R² can then be removed to give primary alcohol (123, R³═Ac, TMS or TBDMS), and that can be oxidized to aldehyde (124, R³═Ac, TMS or TBDMS). Aldehyde (124) is a very useful common intermediate for 1α-hydroxy-20-epi-22-alkenyl Vitamin D analogues. Reaction of aldehyde (124) with an appropriate crotonate anion derivative, such as ylide (83) will give the unsaturated E,E-ester (125, R³═Ac, TMS or TBDMS), which can be converted to 20-epi-Seocalcitol (92) by treatment with excess ethyl lithium which will both form the desired side chain and cleave the protecting groups, or in the case of TBDMS with an additional TBAF treatment to cleave the fluoride. Alternatively, reaction of (124, R³═Ac, TMS or TBDMS) with stabilized ylide (93) will give enone (126), which can be selectively reduced with many known chiral reducing agents to the 24 alcohol (127, R³═Ac, TMS or TBDMS), followed by a simple deacetylation or desilylation to give 20-epi-Calcipotriol (90).

### 5. Synthesis of Vitamin D Derivatives Extended at C21, and at the Normal Steroidal Side Chain

As the above disclosures demonstrate, the processes and intermediates disclosed herein have general utility for the preparation of 20-epi Vitamin D analogues, and of 20-epi steroids with more than a simple allcene functionality in the B-ring. The examples given above are illustrative of the utility of the process and the key intermediates claimed in this patent, and are not meant to limit the methodology. For example, the ready preparation of O-silylpregna-5,7-dien-3β-ol-20-ones exemplified by (20) allows for extension of the normal C17 side chain in both directions off of C20. We have described above the building out of the steroidal side chain via epoxidation-rearrangement in the normal C22-C27 direction, albeit maintaining the unnatural stereochemistry at C20, and whilst leaving C21 as a methyl group. However, compound (20), upon generation of the kinetic enolate (128), which can be done straightforwardly by treatment of compound (20) with LDA at low temperature in solvents such as THF, a process well known to those skilled in the art, activates the C21 methyl towards electrophilic attack. (Konopelslci, J. P., Djerassi, C. J. Med. Chem., 23, 722-6, (1980)). This allows especially for new carbon-carbon bonds to be formed at C21, via the very well established process of enolate alkylation, whilst also regenerating the C20 carbonyl to form a derivative (129). The reformed carbonyl of (129) can then be epoxidized with dimethylsulfonium methylide to give (130), and rearranged with a Lewis acid to the corresponding aldehyde (131), in exactly the same way as is done for converting compound (20) into compounds (29) and (32). Then this new aldehyde (131) can be chain extended as described previously to form formally 20-epi-21-extended steroid derivatives (132). However, as shown below in Scheme 10, depending on the nature of the substituents put on C21 and C22, one can envision that the main “natural” steroid side chain extension on C22 and the “unnatural” C21 extension may be reversed, in which case the product would have the formal “natural” 20R-stereochemistry. In the most extreme case the C22 aldehyde can be reduced directly to the corresponding methyl group, for example, by reduction to the alcohol, tosylation and LiALH₄ reduction, to form the natural 20R,21-methyl side chain. The usual photolyses/thermolysis of (132) will give the Vitamin D triene analogues (133), which can be converted to the corresponding Windhaus-Grandmann ketones (134) as described above.

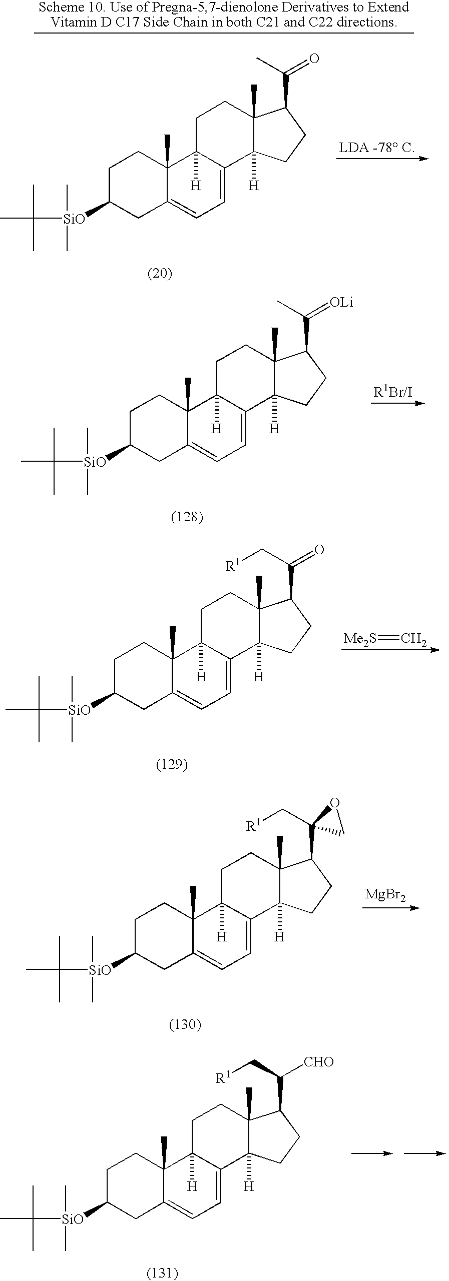

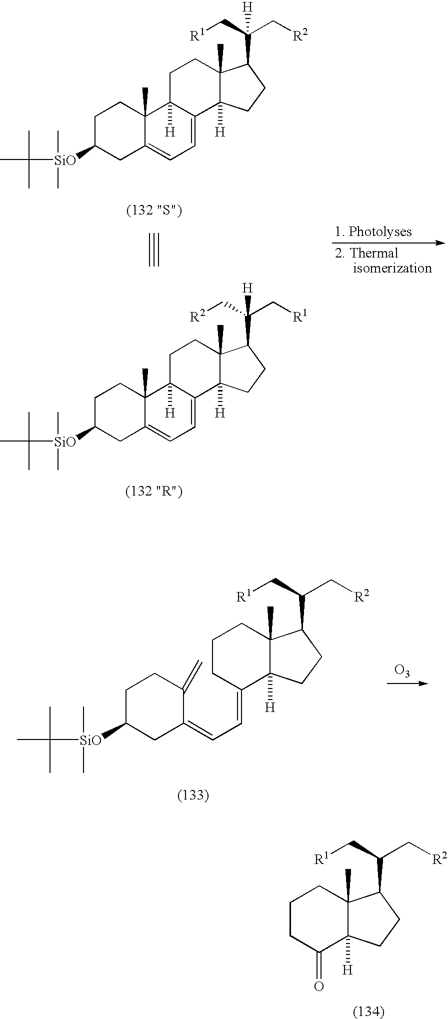

As C21-extended steroids are not readily produced, let alone Vitamin D deriviatives, this invention also applies to the synthesis of C21-extended steroids with both the “natural” 20R, and the “unnatural” 20S configuration, as well as their corresponding B-ring opened trienic Vitamin D analogues. Therefore, the process described in Scheme 10 can also use O-silylpregn-5-en-3-ol-20-ones, such as (7) and (8) to form intermediates such as (135)-(139) which can then be used in conventional steroidal chemistry, as (20-epi)-21-norcholesterol derivatives, as illustrated in Scheme 11.

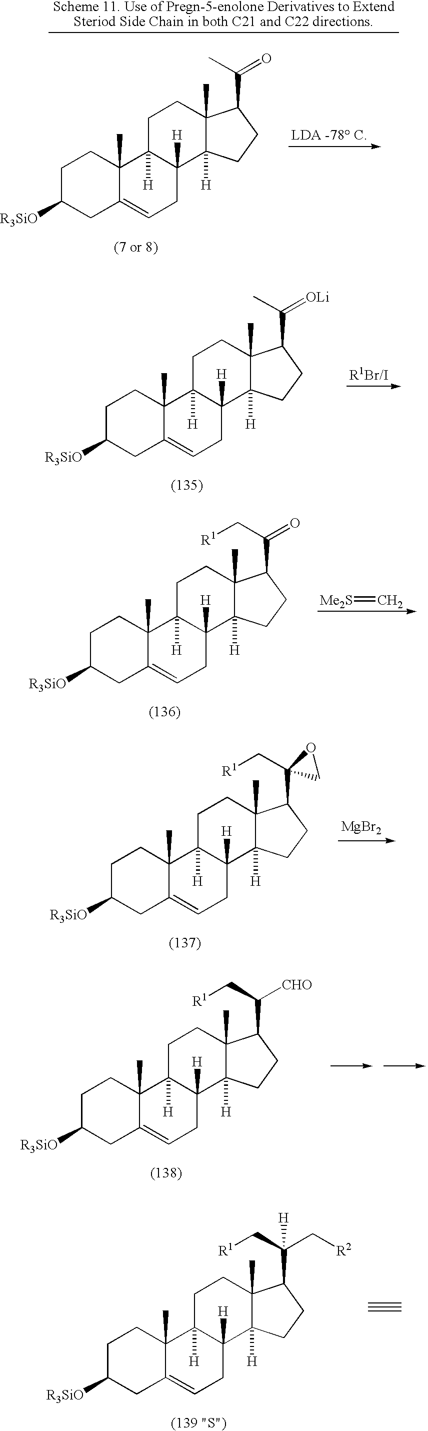

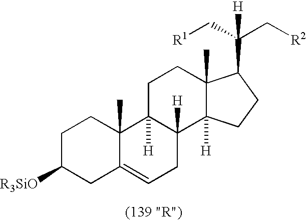

Although the electrophiles reacted with enolates (128) and (135) in the above illustrations are described as alkyl halides, which would obviously include allylic and benzylic halides, there are many other electrophiles, obvious to one skilled in the art, such as aldehydes, ketones, esters, amides and acyl halides, and Michael acceptors which could be used in this process. Additionally, intermediates (136) and (139) can be desaturated to form the corresponding 5,7-dienes, and then converted to Vitamin D derivatives.

As illustrations of possible uses of these obvious extensions of the technology described in this patent application, synthetic schemes to prepare 21,23-bisnor Becocalcidiol (140) and its C20 epimer (141), Schemes 12 and 13, both 21R and 21S epimers of the so-called Gemini Vitamin D derivatives (141) and (142), Schemes 14 and 15, and both 20S and 20R 21-norcholesterols (143) and (144), Schemes 16 and 17, are shown below.

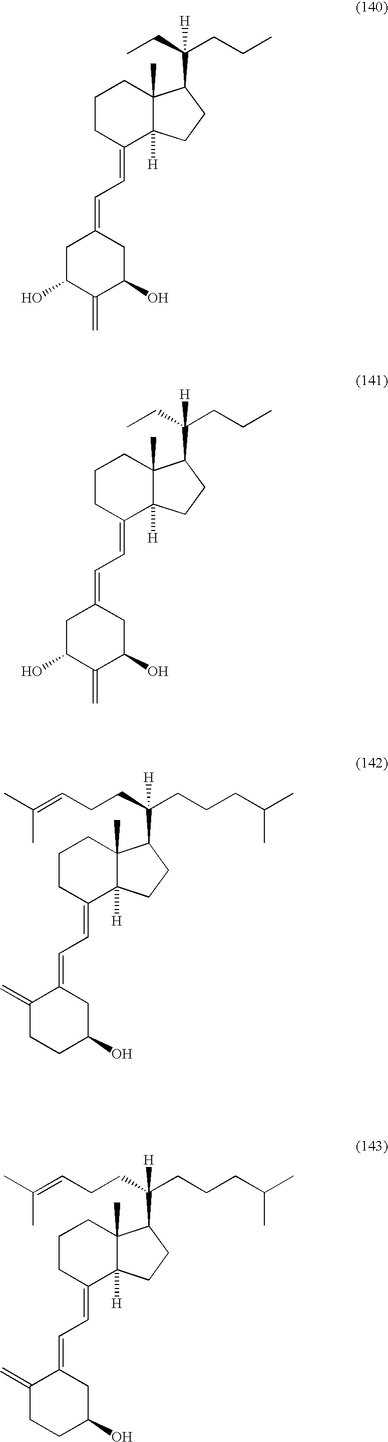

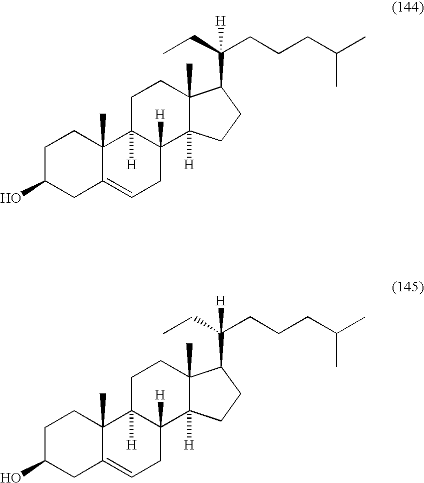

Scheme 12 starts with alkylation of ketone (20) with LDA and methyl iodide, to give ketone (146), which is epoxidized, rearranged with magnesium bromide and reduced to alcohol (147) with NaBH₄. Tosylation and copper-catalysed coupling of ethylmagnesium bromide gives the photolysis precursor (148). Going through the photolysis-isomerization and ozonolysis sequence gives the corresponding Windhaus-Grundmann ketone, which is reduced in situ to alcohol (149). Oxidation of the alcohol, back to the Windhaus-Grundmann ketone, followed by Lythgoe coupling and deprotection, as demonstrated for Becocalcidiol will give its bis-homo analogue (140).

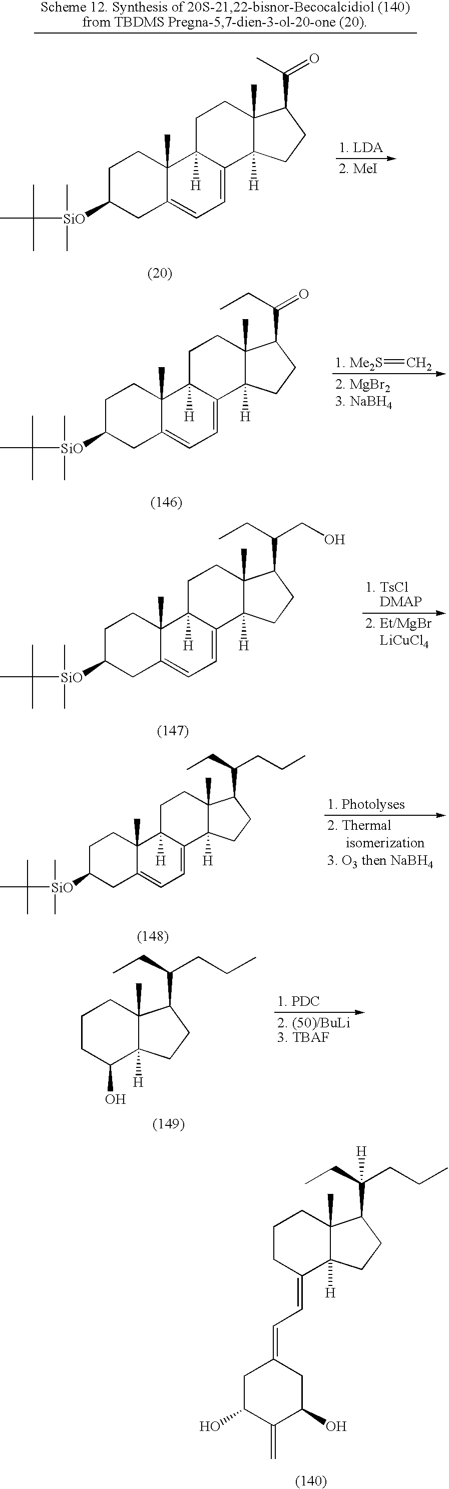

Scheme 13 is very similar to Scheme 12, starting with alkylation of ketone (20) with LDA and ethyl iodide, to give ketone (150). The sequence is continued exactly as in Scheme 12, except that the tosylate derived from alcohol (151) is coupled with methylmagnesium bromide and LiCuCl₄, to form the steroid (152), which is converted as before to bicycloalcohol (153) and Becocalcidiol analogue (141).

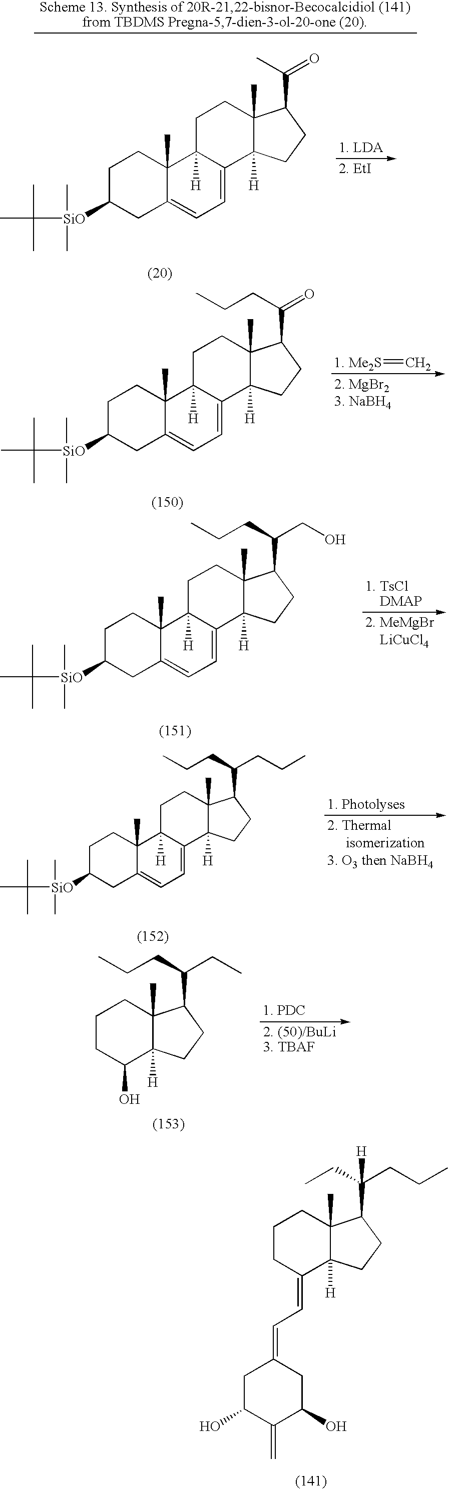

One very interesting variant on normal Vitamin D structures which has been reported is the so-called “Gemini” Vitamin D derivatives, (Adorini, L., Penna, G., Uskovic, R., Maehr, H. WO 2004/098522), where C21 is extended to form a second, natural-like C22-27 side chain. In most of the published cases, the C21 and C22-extended side chains are different from one another, meaning that C20 is a chiral center. Because the methodology described herein allows for complete stereocontrol at C20, it is especially suitable for the efficient synthesis of such compounds, as is illustrated by the synthesis of both C20 isomers of a simple “Gemini” derivative below. In Scheme 14, the enolate of (20) is reacted with prenyl promide to make ketone (154), which is homologated to alcohol (155) as described previously. Reaction of the corresponding tosylate with isopentylmagnesium bromide and copper catalyst gives the steroid (156), which can be converted to the corresponding Vitamin D derivative by the usual photolytic sequence, and then deblocked to give “Gemini” Vitamin D derivative (142) stereospecifically. In Scheme 15 switching the alkylating agent to isopentyl bromide, to give ketone (157) and the Grignard reagent to prenylmagnesium bromide on the tosylate derived from alcohol (158) gives epi-steroid (159), which is photolysed and deblocked to (143).

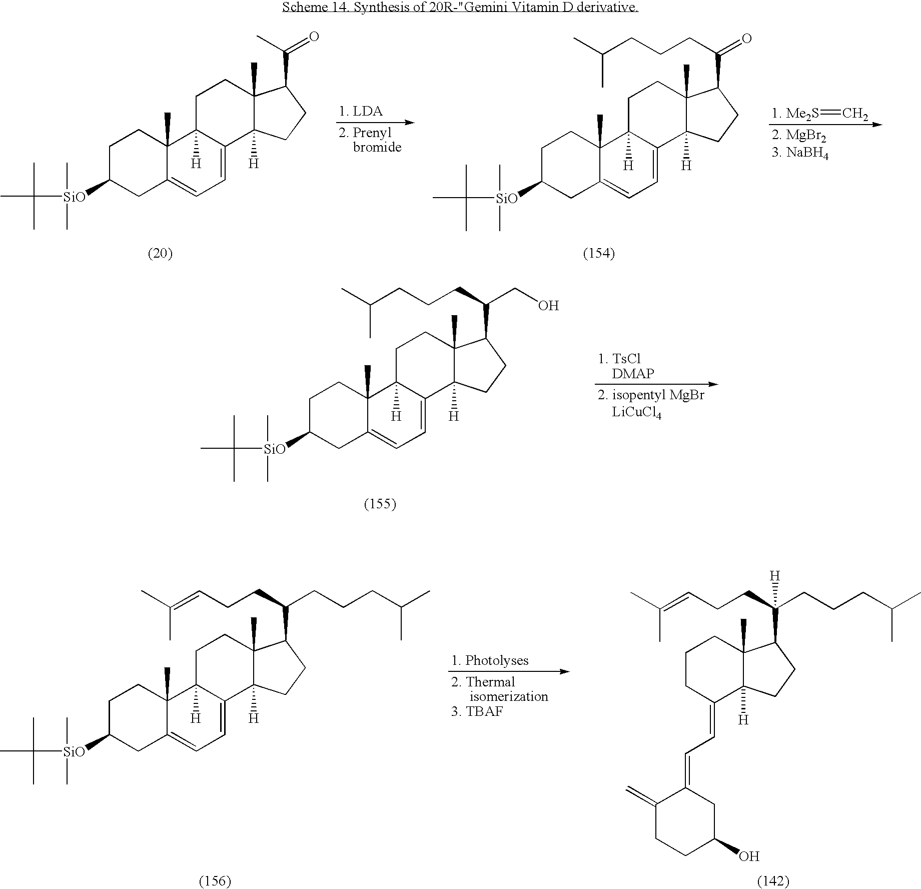

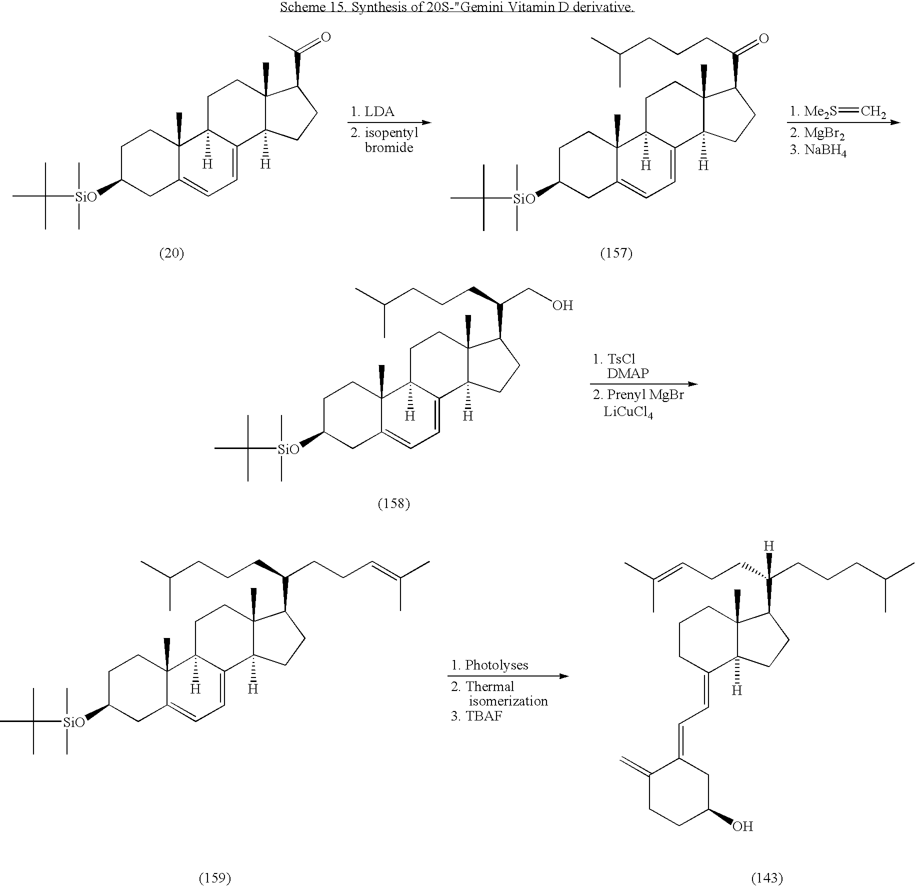

It should be noted that some “Gemini” derivatives contain an A-ring, which will have to be introduced by a Lythgoe coupling to an appropriate Windhaus-Grundmann ketone, and which contain a double or triple bond in one of the “Gemini” side chains. In such cases, an appropriate “orthogonal” protection of the alcohol corresponding to (155) or to (158), followed by photolysis and isomerization, and ozonolysis will give the equivalent of the Imhoffen-Lythgoe ketone, which can be be coupled with the appropriate A-ring synthon, and then the C22 (C22′) alcohol can be selectively deprotected, activated to displacement (or oxidized to the corresponding aldehyde) and chain extended by methods known to one skilled in the art.

The use of either silyl ether (16) or silyl ether (38) to make simple steroid derivatives, homologated at C21 is illustrated in Schemes 16 and 17. For example, silyl ether (16) can be methylated on C21 to give the 21-homopregnenolone (160), which can be epoxidized with dimethylsulfonium methylide to form (161), which can be rearranged to the aldehyde (162). Reaction of aldehyde (162) with an isopentyl Wittig reagent gives the homocholesterol derivative (163), whereupon selective side chain hydrogenation and desilylation gives 20S-21-homocholesterol (144). Using silyl ether (38) in a sequence where the enolate is alkylated with isopenyl bromide, to give (164), followed by epoxidation to (165), rearrangement to aldehyde (166), and methylenation will give the vinyl steroid (167), which can be reduced and deblocked to give 21-homocholesterol (145).

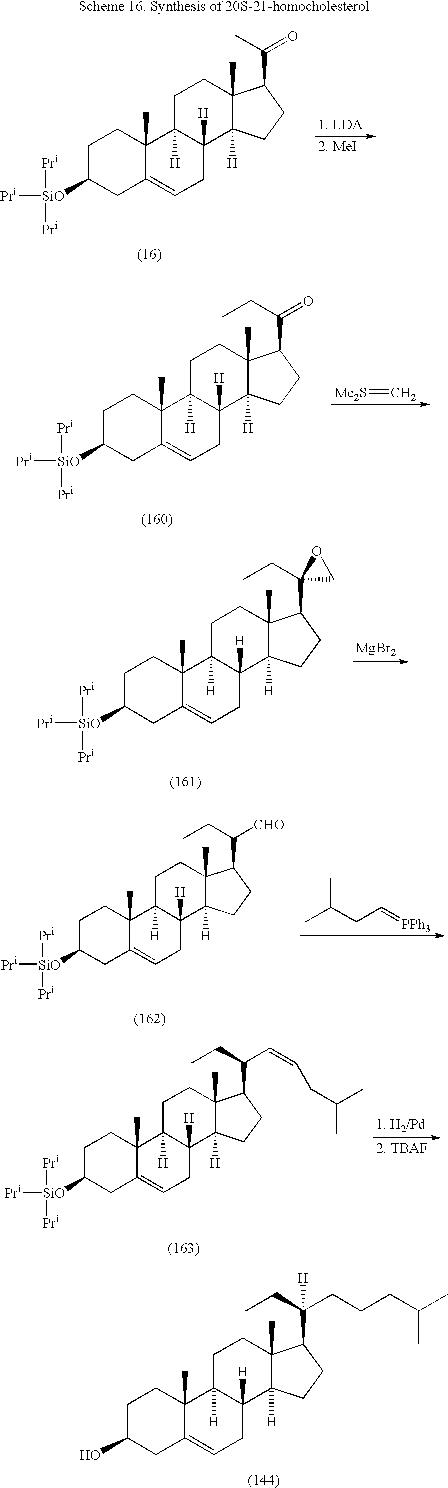

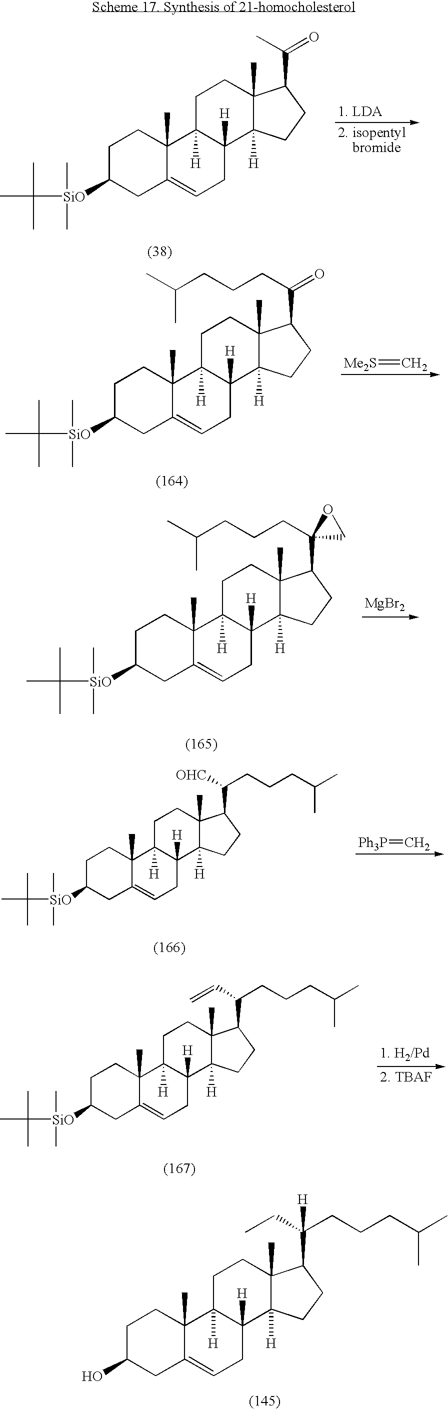

### 6. An Alternative Synthesis of 20-epi-Vitamin D Derivatives

An alternative strategy of removing the A-ring from steroids has been described, by ozonation of steroidal 5-enes, elimination of the 3-oxy substituent, and loss of the A-ring via a Norrish Type II photocleavage, as described by Dauben (Tet. Letters, 35, 2149-52, (1994) and Gao (Tet. Letters, 40, 131-2, (1999). This will be exemplified by two possible preparations of Becocalcidiol (52) from the silyl ethers (15) and (38). In Scheme 18, silyl ether (15) is ozonized, and worked up oxidatively to give ketoacid (168). Treatment of (168) with two equivalents of strong base at low temperature, leads to siloxide elimination to give the enone (169). Photolysis of this compound will lead to cleavage of the A-ring in good yield to give the unsaturated CD-ring acetic acid (170). The double bond is reduced out catalytically, to give acid (171). Hell-Vollhardt-Zelinsky bromination of this acid followed by methanol work-up gives bromoester (172). This can be eliminated with a strong base to give the unsaturated ester (173), which is reduced with LiAlH₄, to the allyl alcohol (174). Tosylation of (174) gives allyl tosylate (175). This can either be displaced with lithium diphenyl phosphide or sodium 2-thiobenzothiazole, followed by oxidation to give allyl phosphine oxide (176) or allyl sulfone (177). Either of these can be treated with butyl lithium or LDA, followed by ketone (178) (DeLuca and Sicinsld, U.S. Pat. No. 5,843,928) to give protected Becocalcidiol analogue (51), which is deprotected to Becocalcidiol (52).

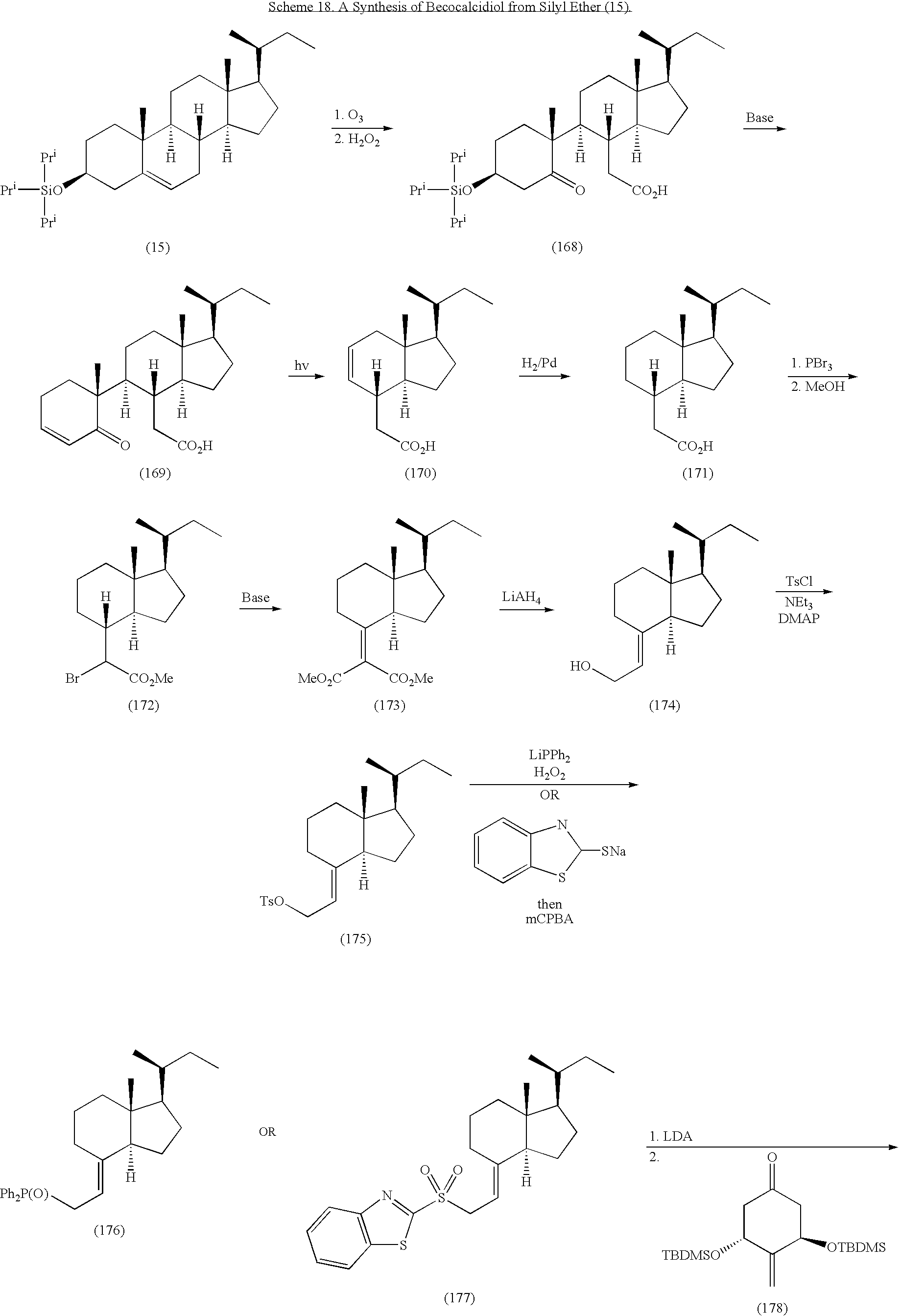

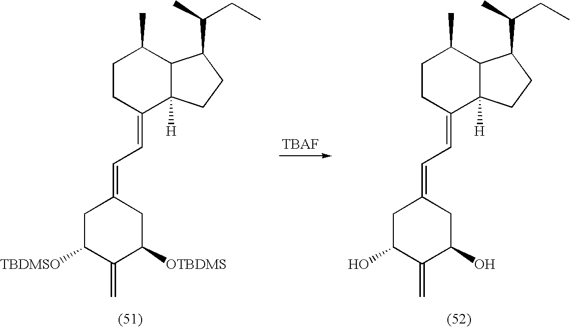

In Scheme 19 the TBDMS ether (38) is ozonized, and worked up reductively, and acetalized as described by Gao, to give acetal (179). This is treated with a strong base to give enone (180), and the A-ring is cleaved photolytically to give bicycloalkene (181), which is reduced to (182). Acetal (182) can be treated with tosylhydrazine and acid to make tosylhydrazone (183) directly. A Bamford-Stevens reaction will give the vinyl compound (184), which can be oxidized to the allyl alcohol (185) by SeO₂, either stoichiometrically or catalytically, with another oxidant such as t-butyl hydroperoxide. The hydrogen at C8 is axial, and other good enophile oxidizing agents such as EtO₂CNSO, should also allow for this conversion. Reaction with a strong base and chlorodiphenylphosphine should produce the allylic phosphenite ester (186) which upon 3,2-rearrangement should give purely the E-isomer (176) shown. Alternatively, treatment of allyl alcohol (185) with benzothiazole-2-sulfenyl chloride will give the allyl sulfenate ester (187) which will rearrange thermally to an allyl sulfoxide, which can be oxidized in situ with mCPBA or other mild oxidants to the allyl sulfone (177). The intermediates (176) and (177) can then be taken onto Becocalcidiol as described in Scheme 18.

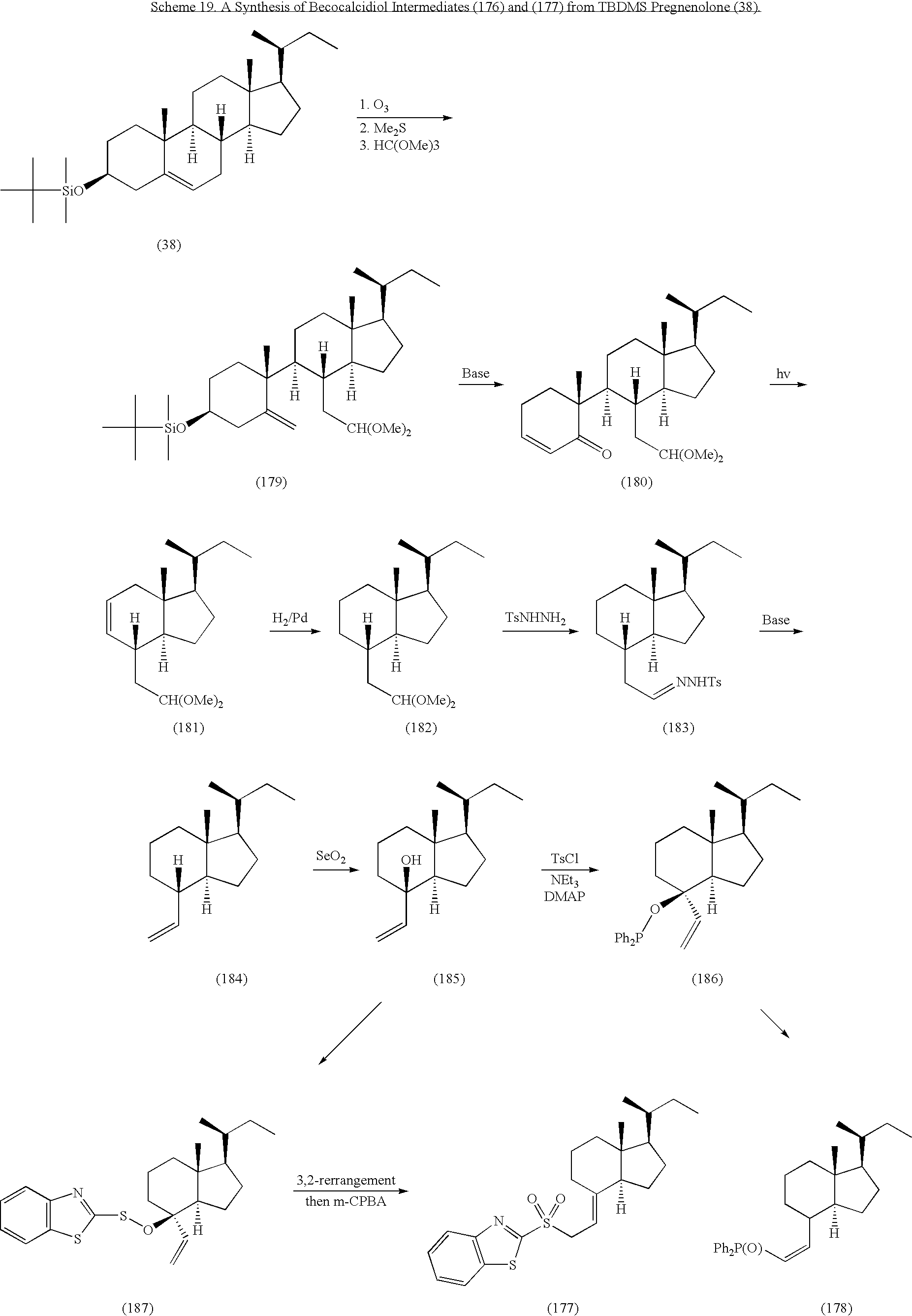

In this disclosure, the term photolysis can be used to describe several different photochemical processes. If the process is simply described as a photolysis, or photolysis/isomerization, to turn a steroidal B-ring 5,7-diene into a Vitamin D derivative, with no further elaboration, it can refer to one of two processes. One involves an initial photolysis at a wavelength of below 300 mM, at temperatures close 0° C., to open the diene to the 6E-Δ_{5-10,6-7,8-9} trienic “preVitamin D” analogue, which usually involves generating a photostationary equilibrium, which includes large amounts, or a preponderance, of the corresponding 6E-stereoisomer, the “Tachysterol” analogue. This is followed by a second irradiation at longer wavelength, preferably around 350 nM, for example using a uranium glass filter, to isomerize most of the Tachysterol analogue back to the desired “preVitamin D” analogue, and is then followed by a thermal 1,7-hydride shift to give the desired 5Z,7E-Δ_{10-19,5-6,7-8} trienic “Vitamin D” analogue. The second process involves a descending film photolysis technique carried out at room temperature or above, in a specialized photolysis apparatus, which allows for the ring opening and 1,7-hydride shift to be done, producing a preponderance of the desired 5Z,7E-Δ_{10-19,5-6,7-8} trienic “Vitamin D” analogue in a single pass. If photolysis to produce the 6E-Δ_{5-10,6-7,8-9} trienic “preVitamin D” analogue is specifically described, depending on the context, it will refer either to a single shorter wavelength photolysis, which is understood to produce a 6E-Δ_{5-10,6-7,8-9} trienic “preVitamin D”/6Z-Δ_{5-10,6-7,8-9} trienic “tachysterol” analogue mixture, or the short wavelength photolysis, followed by the longer wavelength 6Z to 6E deequilibration photolysis, and in each case done at low enough temperatures to suppress the 1,7-hydride shift to the 5Z,7E-Δ_{10-19,5-6,7-8} trienic “Vitamin D” analogue.

### EXPERIMENTALS

The invention is illustrated further by the following examples, which are not to be construed as limiting the invention in scope or spirit to the specific procedures described in them. Those having skill in the art will recognize that the starting materials may be varied and additional steps employed to produce compounds encompassed by the invention, as demonstrated by the following examples. Those skilled in the art will also recognize that it may be necessary to utilize different solvents or reagents to achieve some of the above transformations. In some cases, protection of reactive functionalities may be necessary to achieve the above transformations. In general, such need for protecting groups, as well as the conditions necessary to attach and remove such groups, will be apparent to those skilled in the art of organic synthesis. When a protecting group is employed, deprotection step may be required. Suitable protecting groups and methodology for protection and deprotection such as those described in Protective Groups in Organic Synthesis by T. Greene and P. Wuts are well known and appreciated in the art.

Unless otherwise specified, all reagents and solvents are of standard commercial grade and are used without further purification. The appropriate atmosphere to run the reaction under, for example, air, nitrogen, hydrogen, argon and the like, will be apparent to those skilled in the art.

### Example 1

### 3,O-(t-Butyldimethylsilyl)pregn-5-en-3β-ol-20-one

Pyridine (4.0 mL) was added in one portion to a vigourously stirred suspension of 3β-pregn-5-en-20-one (6.33 g, 20 mmol) and 4-(N,N-dimethylamino)pyridine (0.244 g, 2.0 mmol) in DMF (40 mL) containing t-butyldimethylsilyl chloride (3.77 g, 25 mmol) under nitrogen at 25° C. After 20 h, the reaction mixture was stirred on an ice-bath for 1 h, and then Buchner filtered through a glass frit. The residue was rinsed with cold DMF (2×20 mL), and was dried in a vacuum oven at 60° C. for 5 h, to give 3β-(t-butyldimethylsiloxy)pregn-5-en-20-one (8.46 g) as a white crystalline solid, containing 0.54% DMF by weight. Yield=97.7%. ¹H NMR (CDCl₃ 500 MHz) δ: 0.086 (6H, s), 0.654 (3H, s), 0.916 (9H, s), 0.92-1.05 (1H, m), 1.026 (3H, s), 1.06-1.33 (3H, m), 1.45-1.76 (9H, m), 1.82-1.86 (1H, brd), 2.00-2.15 (2H, m), 2.151 (3H, s), 2.21-2.30 (3H, m), 2.558 (1H, t, J=9.0 Hz), 3.509 (1H, approx septet, J=4.6 Hz), 5.328 (1H, brd, J=5.0 Hz).

### Example 2

### 3β-(Triisopropylsiloxy)pregn-5-en-20-one

To a suspension of pregnenolone (6.28 g, 19.8 mmol) in DMF (20 mL) and DCM (20 mL) at 25° C. was added imidazole (2.7 g, 39.7 mmol) followed by triisopropylsilyl chloride (5.5 mL, 25.8 mmol). The mixture became homogeneous after a few hours and was stirred for 24 h. The solution was partitioned between EtOAc and water, and extracted with EtOAc (2×), washed with sat. sodium bicarbonate, water, brine, dried over magnesium sulfate, and concentrated to give 12.9 g of a crude white solid. Recrystallization from isopropanol afforded 5.98 g of the title compound. A second crop of 0.95 g (identical by ¹H NMR) was obtained from the mother liquor for a combined yield of 74%. ¹H NMR (CDCl₃ 500 MHz) δ: 0.654 (3H, s), 0.92-1.33 (4H, m), 1.033 (3H, s), 1.139 (21H, s), 1.43-1.76 (8H, m), 1.82-1.88 (2H, m), 1.96-2.10 (2H, m), 2.150 (3H, s), 2.21-2.34 (3H, m), 2.558 (1H, t, J=9.0 Hz), 3.586 (1H, approx septet, J=4.6 Hz), 5.344 (1H, brs).

### Example 3

### 3β-(t-Butyldimethylsiloxy)-22-homopregn-5-en-20R,22-epoxide

A slurry of potassium hexamethyldisilazane (4.01 g, 20 mmol) and trimethylsulfonium iodide (4.08 g, 20 mmol) in THF (20 mL) was stirred under nitrogen at 25° C. for 10 minutes to form a very pale yellow slurry. Then toluene (20 mL) was added and the mixture was cooled to −70° C. on a dry ice/isopropanol bath for 20 minutes. Then a solution of 3β-(t-butyldimethylsiloxy)pregn-5-en-20-one (4.19 g, 9.73 mmol) in toluene (60 mL) was added dropwise over 45 minutes. The reaction was allowed to stir at −70° C. for another hour, and was then allowed to warm slowly to −60° C. over 1 hour and to −5° C. over another hour. The reaction was quenched by the rapid addition of acetic acid (2.0 mL) forming a much thicker slurry. Water (100 mL) and NaHSO₃ (0.10 g) were added with rapid stirring, and the phases were separated. The aqueous phase was extracted with MTBE (2×50 mL), and the combined organic extracts were washed with water (2×50 mL), saturated aqueous sodium bicarbonate solution (50 mL) and saturated brine (50 mL), and dried (MgSO₄). The solvent was removed rigorously under reduced pressure to give crude 3β-(t-butyldimethylsiloxy)-22-homopregn-5-en-20R,22-epoxide (4.12 g, 95%) as a free flowing white solid, which NMR analysis showed to contain a 44:1 ratio of the desired and undesired C20 epimers. ¹H NMR (CDCl₃ 500 MHz) δ: 0.084 (6H, s), 0.838 (3H, s), 0.916 (9H, s), 0.95-1.05 (1H, m), 1.05-1.10 (1H, m), 1.12-1.16 (1H, d of d of t), 1.407 (3H, s), 1.41-1.66 (11H, m), 1.571 (3H, s), 1.716 (2H, brt), 1.80-1.84 (1H, brd), 1.97-2.03 (1H, brd), 2.18-2.23 (1H, brd), 2.26-2.32 (1H, brt), 2.352 (1H, d, J=4.9 Hz), 2.527 (1H, d, J=4.9 Hz), 3.506 (1H, approx septet, J=˜5.0 Hz), 5.339 (1H, brd, J=5.2 Hz).

### Example 4

### 3β-(Triisopropylsiloxy)-22-homopregn-5-en-20R,22-epoxide

A stirred suspension of potassium hexamethyldisilazane (7.3 g, 36.7 mmol) in THF (50 mL) was cooled to −5° C. in a dry ice/isopropanol bath under nitrogen. Trimethylsulfonium iodide (7.5 g, 36.7 mmol) was added in one portion and the mixture was stirred 15 min. After cooling to −65° C., a solution of 3β-(triisopropylsiloxy)pregn-5-en-20-one (6.95 g, 14.7 mmol) in THF (20 mL) was added dropwise over 20 min. The mixture was stirred for 3 h, then allowed to warm slowly to room temperature and stirred 30 min. The mixture was cooled in an ice bath, quenched with 0.2 M citric acid (50 mL), and then allowed to warm to room temperature and stirred for 15 min. The mixture was partitioned between EtOAc and water, extracted with EtOAc (2×), washed with dilute aqueous sodium thiosulfate, brine, dried over magnesium sulfate, and concentrated to give 3β-(triisopropylsiloxy)-22-homopregn-5-en-20R,22-epoxide (7.16 g, 100%) as white plates with a 40:1 20R:S ratio (by ¹H NMR). ¹H NMR (CDCl₃ 500 MHz) δ: 0.838 (3H, s), 0.946 (1H, sl brd of t, J_{d}=5 Hz, Jₜ=11 Hz), 1.041 (3H, s) 1.083 (21H, s), 1.05-1.10 (1H, m), 1.257 (1H, d of t, J_{d}=5 Hz, Jₜ=12 Hz), 1.406 (3H, s), 1.41-1.66 (8H, m), 1.578 (3H, s), 1.734 (1H, t, J=9.5 Hz), 1.80-1.88 (2H, m), 1.97-2.03 (1H, brd), 2.192 (1H, d of d of d, J=2.5, 5, 13 Hz), 2.26-2.32 (1H, brt), 2.352 (1H, d, J=4.8 Hz), 2.527 (1H, d, J=4.8 Hz), 3.580 (1H, approx septet, J=5.0 Hz), 5.339 (1H, brs).

### Example 5

### 20R,3 β-(t-Butyldimethylsiloxy)-22-homopregn-5-en-22-al

A slurry of magnesium bromide bis(diethyl etherate) (101.3 mg, 0.40 mmol) in toluene (5 mL) was added dropwise over 1 minute to a solution of crude 3 β-(t-butyldimethylsiloxy)-22-homopregn-5-en-20R,22-epoxide (889.8 mg, 2.0 mmol) in toluene (20 mL), stirred under nitrogen at 0° C. The initial cloudy mixture gradually became a clear solution with a very fine white precipitate. After 4 hours, the reaction mixture was capped, and was placed in a 4° C. refrigerator for 45 hours. The cold solution was quenched with dilute hydrochloric acid (0.1 M, 10 mL), and the phases were separated. The aqueous phase was extracted with MTBE (10 mL), and the combined organic phases were washed with water (10 mL), saturated brine (10 mL) and dried (MgSO₄). The solvent was removed rigorously under reduced pressure to give 842 mgs of white slightly waxy solid, which nmr analysis showed to contain a 20:1 ratio of 20R:S aldehyde. This material was recrystallized from acetone at 0° C. to give 20R,3 β-(t-butyldimethylsiloxy)-22-homopregn-5-en-22-al (625.8 mg, 70.3%) as white plates with a 65:1 20R:S ratio. A further recrystallization from acetone at 0° C. gave the desired aldehyde (490.3 mg, 55.1%) as white rods with a ≧250:1 20R:S ratio. Combining the second crop from the first recrystallization and the mother liquors from the second recrystallization (203 mg) and recrystallizing this material twice more from acetone at 0° C., gave further aldehyde (96.0 mg, 10.8%) as white rods with a 100:1 20R:S ratio. ¹HNMR (CDCl₃ 500 MHz) δ: 0.079 (6H, s), 0.711 (3H, s), 0.911 (9H, s), 0.947 (1H, d of t, J_{d}=5 Hz, Jₜ=11 Hz), 1.012 (3H, s), 1.059 (3H, D, J=6.8 Hz), 1.01-1.21 (5H, m), 1.34-1.77 (9H, m), 1.80-1.85 (1H, br d of t), 1.86-1.95 (1H, m), 1.97-2.05 (1H, m), 2.17-2.22 (1H, brd), 2.25-2.38 (2H, m), 3.497 (1H, approx septet, J=4.6 Hz), 5.337 (1H, narrow m), 9.570 (1H, D, J=5.0 Hz).

### Example 6

### 20R,3β-(Triisopropylsiloxy)-22-homopregn-5-en-22-al

A stirred solution of 3β-(triisopropylsiloxy)-22-homopregn-5-en-20R,22-epoxide (1.30 g, 2.67 mmol) in toluene (8 mL) was cooled in an ice bath under nitrogen. A solution of magnesium bromide in ether (3.1 mL, 0.53 mmol, 0.17 M) was added, and the solution was allowed to warm to room temperature and stirred for 3 h. The solution was partitioned between EtOAc and 0.5 M HCl, extracted with EtOAc (2×), washed with sat. sodium bicarbonate, brine, dried over magnesium sulfate, and concentrated to give 1.35 g of a white solid. Recystallization from isopropanol afforded 20R,3β-(triisopropylsiloxy)-22-homopregn-5-en-22-al (0.85 g, 65%) as white needles with a 30:1 20R:S ratio by ¹H NMR. Further crystallization improved the 20R:S ratio to 65:1 in overall 50% yield, also determined by ¹H NMR ¹H NMR (CDCl₃ 500 MHz) δ: 0.712 (3H, s), 0.947 (1H, d of t, J_{d}=5 Hz, Jₜ=11 Hz), 1.019 (3H, s), 1.059 (3H, d, J=6.9 Hz), 1.078 (21H, s), 1.01-1.21 (5H, m), 1.34-1.74 (8H, m), 1.78-1.94 (3H, m), 1.97-2.05 (1H, br d of t), 2.25-2.38 (3H, m), 3.567 (1H, approx septet, J=4.6 Hz), 5.333 (1H, sl brd J=4.8 Hz), 9.570 (1H, d, J=5.0 Hz).

### Example 7

### 20S,3β-(t-Butyldimethylsiloxy)-22,23-bishomopregna-5,22-diene

n-Butyl lithium (2.5 M in hexanes, 0.65 mL, 1.625 mmol) was added dropwise over 2 minutes to a light yellow suspension of methyltriphenylphosphonium iodide (608 mg, 1.5 mmol) in THF (5 mL) stirred under nitrogen at 0° C. After 10 minutes 20R,3 β-(t-butyldimethylsiloxy)-22-homopregn-5-en-22-al (222.4 mg, 0.50 mmol) was added in one portion to the reaction mixture. After 30 minutes at 0° C., celite (1 g) and hexanes (40 mL) were added to the reaction mixture, which was stirred at 0° C. for a further 30 minutes, before vacuum filtration through a short silica gel plug. The plug was rinsed with 4% MTBE in hexanes (50 mL) and the combined filtrates were concentrated rigorously under reduced pressure to give 20S,3β-(t-butyldimethylsiloxy)-22,23-bishomopregna-5,22-diene (219.5 mg, 99.1%) as glistening white plates with a ≧200:1 20R:S ratio by ¹H NMR. ¹H NMR (CDCl₃ 500 MHz) δ: 0.081 (6H, s), 0.688 (3H, s), 0.913 (9H, s) 0.948, (3H, d, J=6.6 Hz), 1.034 (3H, s), 0.089-1.21 (6H, m), 1.26-1.34 (1H, brq), 1-36-1.65 (6H, m), 1.70-1.77 (1H, m), 1.78-1.88 (2H, m), 1.96-2.04 (2H, m), 2.07-2.16 (1H, m), 2.18 (1H, brd of d of d), 2.28 (1H, brt), 3.499 (1H, septet, J=4.9 Hz), 4.859 (1H, d of d, J=1.810.1 Hz), 4.958 (1H, d of d, J=1.8, 17.2 Hz), 5.338 (1H, sl brd J=5.1 Hz), 5.718 (1H, d of t, J_{d}=17.2 Hz, Jₜ=10.1 Hz).

### Example 8

### 20S,3β-(Triisopropylsiloxy)-22,23-bishomopregna-5,22-diene

A stirred suspension of methyltriphenylphosphonium iodide (747 mg, 1.85 mmol) in THF (5 mL) was cooled in an ice bath under nitrogen. Butyllithium (0.69 mL, 1.72 mmol, 2.5 M in hexane) was added dropwise and the resulting orange mixture was stirred for 20 min. 20R,3β-(Triisopropylsiloxy)-22-homopregn-5-en-22-al (290 mg, 0.60 mmol) was added in one portion, and the mixture was allowed to warm to room temperature and stirred for 2 h. The mixture was poured into hexane (25 mL) and stirred for 15 min, then filtered through a pad of magnesium sulfate, and rinsed with hexane. The filtrate was concentrated to give 300 mg of a crude white solid. Flash chromatography (1-2% EtOAc/hexanes) gave 20S,3β-(triisopropylsiloxy)-22,23-bishomopregna-5,22-diene (245 mg, 85%) as a white solid with a 63:1 20S:R ratio determined by ¹H NMR. ¹H NMR (CDCl₃ 500 MHz) δ: 0.690 (3H, s), 0.923 (1H, d of t, J_{d}=4.7 Hz, Jₜ=11.2 Hz), 0.949, (3H, d, J=6.6 Hz), 1.018 (3H, s), 1.081 (21H, s), 1.01-1.21 (5H, m), 1.26-1.34 (1H, brq), 1.36-1.74 (6H, m), 1.78-1.90 (3H, m), 1.97-2.05 (2H, m), 2.07-2.16 (1H, m), 2.22-2.36 (2H, m), 3.573 (1H, approx septet, J=4.6 Hz), 4.859 (1H, d of d, J=1.510.1 Hz), 4.959 (1H, d of d, J=1.5, 17.1 Hz), 5.335 (1H, sl brd J=5.0 Hz), 5.718 (1H, d of t, J_{d}=17.1 Hz, Jₜ=10.1 Hz).

### Example 9

### 20S,3 β-(t-Butyldimethylsiloxy)-22,23-bishomopregn-5-ene

A slowly stirred slurry of 5% Pd/C (19 mg) and 20S,3β-(t-butyldimethylsiloxy)-22,23-bishomopregna-5,22-diene (182.4 mg, 0.412 mmol) in THF/MeOH (2:1, 6 mL) was put through 4 cycles of vacuum degassing and reconstitution with a hydrogen atmosphere. The mixture was then stirred rapidly under hydrogen for 3 hours at 25° C. The hydrogen was vented, and the reaction mixture, which appeared to have precipitated extensively, was filtered under vacuum through a 1.5×1.5 cm celite plug, and the plug was rinsed with 4% MTBE in hexanes (50 mL) The solvent was stripped rigorously under reduced pressure to give 20S,3β-(t-butyldimethylsiloxy)-22,23-bishomopregn-5-ene (181.9 mg, 99.2%) as fine white plates, with about 10% of the starting alkene still present. ¹H NMR (CDCl₃ 500 MHz) δ: 0.083 (6H, s), 0.695 (3H, s), ), 0.837 (3H, d, J=6.6 Hz), 0.854 (3H, t, J=7.2 Hz), 0.914 (9H, s) 1.038 (3H, s), 0.98-1.24 (5H, m), 1.26-1.34 (2H, m), 1.38-1.65 (9H, m), 1.72-1.86 (3H, m), 1.95-2.05 (2H, m), 2.19 (1H, brd), 2.28 (1H, brt), 3.505 (1H, approx septet, J=4.6 Hz), 5.339 (1H, sl brd J=5.0 Hz).

### Example 10

### 20S,3β-(Triisopropylsiloxy)-22,23-bishomopregn-5-ene

A slurry of 5% Pd/C (20 mg) and 20S,3β-(triisopropylsiloxy)-22,23-bishomopregna-5,22-diene (95 mg, 0.196 nmol) in THF/MeOH (2:1, 3 mL) was put through 3 cycles of vacuum degassing and reconstitution with a hydrogen atmosphere. The mixture was then stirred rapidly under hydrogen for 4 hours at 25° C. The hydrogen was vented, and the reaction mixture was filtered under vacuum through a small plug of anhydrous MgSO₄, rinsing with 10% EtOAc in hexanes. The solvent was stripped rigorously under reduced pressure to give 20S,3β-(triisopropylsiloxy)-22,23-bishomopregn-5-ene (81 mg, 85%) as a light yellow. ¹H NMR (CDCl₃ 500 MHz) δ: ¹H NMR (CDCl₃ 500 MHz) δ: 0.697 (3H, s), 0.838 (3H, d, J=6.6 Hz), 0.855 (3H, t, J=7.2 Hz), 0.940 (1H, d of t, J_{d}=5.2 Hz, Jₜ=11.6 Hz), 1.033 (3H, s), 1.063 (21H, s), 0.98-1.21 (5H, m), 1.26-1.34 (1H, brq), 1.38-1.65 (8H, m), 1.77-1.86 (3H, m), 1.95-2.05 (2H, m), 2.26-2.36 (2H, m), 3.580 (1H, approx septet, J=4.6 Hz), 5.339 (1H, sl brd J=5.0 Hz).

### Example 11

### 20R,3β-(t-Butyldimethylsiloxy)-22-homopregn-5-en-22-ol

20R,3β-(t-Butyldimethylsiloxy)-22-homopregn-5-en-22-al (613 mg, 1.38 mmol, 11.75:1 20R:S ratio) was dissolved with warming in ethanol/toluene (2:1, 9 mL), and the solution was stirred on an ice bath under nitrogen for 10 minutes, producing a fine precipitate. Sodium borohydride (50.0 mg, 1.32 mmol) was added in one portion, and within 1 minute solution had clarified, with mild gas evolution. After 20 minutes aqueous sodium hydroxide (0.25 M, 10 mL) and MTBE (10 mL) were added to the cold mixture. The phases were separated, and the aqueous phase was extracted with MTBE (2×10 mL). The combined organic extracts were washed with water (2×10 mL), saturated brine (10 mL) and dried (MgSO₄). The solvent was removed under reduced pressure to give crude product as a white solid (574 mg). The material was purified by flash chromatography on silica gel, eluting with 5% then 7.5% ethyl acetate/hexanes to give 20R,3β-(t-butyldimethylsiloxy)-22-homopregn-5-en-22-ol (388.2 mg, 62.9%) as a white solid with a ≧200:1 20R:S ratio by ¹H NMR.

¹H NMR (CDCl₃ 500 MHz) δ: 0.082 (6H, s), 0.726 (3H, s), 0.914 (9H, s), 0.984 (3H, d, J=6.5 Hz), 1.026 (3H, s), 0.91-1.30 (5H, m), 1.32-1.42 (1H, m), 1.43-1.68 (9H, m), 1.71-1.78 (1H, m), 1.81-1.88 (2H, m), 1.90-1.95 (1H, brd), 1.97-2.05 (1H, brd), 2.17-2.22 (1H, brd), 2.25-2.34 (1H, brt), 3.46-3.56 (2H, m), 3.73-3.80 (1H, brd of d), 5.343 (1H, brd, J=4.5 Hz).

### Example 12

### One flask, 2 step, preparation of 20R,3β-(t-butyldimethylsiloxy)-22-homopregn-5-en-22-ol from 3β-(t-butyldimethylsiloxy)-22-homopregn-5-en-20R,22-epoxide

A fine suspension of magnesium bromide bis(diethyl etherate) complex (419 mg. 1.62 mmol) in toluene (10 mL) was added dropwise over 10 minutes to a solution of crude 3 p-(t-butyldimethylsiloxy)-22-homopregn-5-en-20R,22-epoxide (1.4455 g, 3.25 mmol) in toluene (30 mL), stirred under nitrogen at −10° C., forming a cloudy suspension. After 1.5 h, the reaction mixture was allowed to warm up slowly to 0° C., and after 5 hours lithium aluminum hydride (75.6 mg. 1.99 mmol was added in one portion, followed by dropwise addition of THF (5 mL) over 2 minutes. After a further 10 minutes at 0° C., the reaction mixture was quenched by addition of dilute hydrochloric acid (CAUTION!, 0.4 M, 25 mL), the first 1 mL being added dropwise, and the remainder only after gas evolution had ceased. The phases were separated, and the aqueous phase was extracted with MTBE (2×25 mL), and the combined organic extracts were rinsed with water (2×25 mL), saturated brine (25 mL) and dried (MgSO₄). The solvent was removed under reduced pressure to give the crude alcohol (1.3516 g), which was purified by flash chromatography on silica gel, eluting with 7.5%, then 10% ethyl acetate in hexanes to give 20R,3β-(t-butyldimethylsiloxy)-22-homopregn-5-en-22-ol (1.1546 g, 79.5%) as glistening white plates with a ≧200:1 20R:S ratio by ¹H NMR.

### Example 13

### 20R,3β-(Triisopropylsiloxy)-22-homopregn-5-en-22-ol

A stirred solution of 20R,3β-(triisopropylsiloxy)-22-homopregn-5-en-22-al (423 mg, 0.87 mmol) in THF (5 mL) and MeOH (1 mL) was cooled in an ice bath under nitrogen. Sodium borohydride (33 mg, 0.87 mmol) was added in one portion and the mixture was stirred for 30 min at 0° C. After quenching with 0.5 M hydrochloric acid, the mixture was partitioned between EtOAc and water, extracted with EtOAc (2×), washed with brine, dried over magnesium sulfate, and concentrated to give 414 mg of a crude white foam. Flash chromatography (15% EtOAc/hexanes) gave 20R,3β-(triisopropylsiloxy)-22-homopregn-5-en-22-ol (313 mg, 74%) as a white crystalline solid with a >100:1 20R:S ratio by ¹H NMR. ¹H NMR (CDCl₃ 500 MHz) δ: 0.727 (3H, s), 0.947 (1H, d of t, J_{d}=5 Hz, Jₜ=11 Hz), 0.987 (3H, d, J=6.5 Hz), 1.033 (3H, s), 1.082 (21H, s), 1.00-1.30 (4H, m), 1.33-1.41 (1H, m), 1.43-1.68 (9H, m), 1.78-1.88 (3H, m), 1.90-1.95 (1H, brd), 1.96-2.04 (1H, brd), 2.23-2.34 (2H, m), 3.512 (1H, brt, J=7.9 Hz), 3.577 (1H, approx septet, J=5.3 Hz), 3.761 (1H, brd, J=10 Hz), 5.338 (1H, brd, J=4.9 Hz).

### Example 14

### 20R,3 D-(Triisopropylsiloxy)-22-homopregn-5-en-22-yl methanesulfonate

A stirred solution of 20R,3β-(triisopropylsiloxy)-22-homopregn-5-en-22-ol (0.44 g, 0.90 mmol) in DCM (5 mL) and triethylamine (0.38 mL, 2.70 mmol) was cooled in an ice bath under nitrogen. Methanesulfonyl chloride (0.10 mL, 1.35 mmol) was added dropwise and the solution was stirred for 2 h at 0° C. The reaction mixture was partitioned between EtOAc and water, extracted with EtOAc (2×), washed with 0.5 M HCl, sat. sodium bicarbonate solution and saturated brine, dried over magnesium sulfate, and concentrated to give 0.52 g of a gummy white foam. Flash chromatography (20% EtOAc/hexanes) gave 20R,3β-(triisopropylsiloxy)-22-homopregn-5-en-22-yl methanesulfonate (0.42 g, 82%) as a white foam, with a ≧200:1 20R:S ratio (by ¹H NMR). ¹H NMR (CDCl₃ 500 MHz) δ: 0.739 (3H, s), 0.946 (1H, d of t, J_{d}=5 Hz, Jₜ=11.4 Hz), 1.028 (3H, s), 1.029 (3H, d, J=6.5 Hz), 1.081 (21H, s), 1.00-1.70 (13H, m), 1.78-1.93 (4H, m), 1.95-2.03 (1H, brd), 2.23-2.35 (2H, m), 3.029 (3H, s), 3.578 (1H, approx septet, J=5.3 Hz), 4.007 (1H, d of d, J=7.8, 9.3 Hz), 4.401 (1H, d of d, J=3.6, 9.4 Hz), 5.332 (1H, brd, J=5.0 Hz).

### Example 15

### 20R,3 β-(Triisopropylsiloxy)-22-homopregn-5-en-22-yl p-toluenesulfonate

To a stirred solution of 20R,3β-(triisopropylsiloxy)-22-homopregn-5-en-22-ol (305 mg, 0.62 mmol) in DCM (5 mL) was added triethylamine (0.26 mL, 1.87 mmol) and a crystal of dimethylaminopyridine under nitrogen at 25° C. Toluenesulfonyl chloride (178 mg, 0.94 mmol) was added and the solution was stirred for 18 h. The solution was partitioned between EtOAc and 0.5 M hydrochloric acid, and was extracted with EtOAc (2×), washed with 5% aqueous sodium hydroxide solution, saturated brine, and dried over magnesium sulfate. The solvent was removed under reduced pressure to give 404 mg of an off-white solid. Recrystallization from isopropanol gave 20R,3β-(triisopropylsiloxy)-22-homopregn-5-en-22-yl p-toluenesulfonate (346 mg, 86%) as white needles with a ≧200:1 20R:S ratio (by ¹H NMR). ¹H NMR (CDCl₃ 500 MHz) δ: 0.623 (3H, s), 0.905 (3H, d, J=6.5 Hz), 1.012 (3H, s), 1.084 (21H, s), 0.87-1.67 (14H, m), 1.78-1.93 (4H, m), 1.93-2.00 (1H, brd), 2.23-2.35 (2H, m), 2.480 (3H, s), 3.576 (1H, approx septet, J=5.3 Hz), 3.836 (1H, t, J=8.3 Hz), 4.165 (1H, d of d, J=3.2, 9.3 Hz), 5.322 (1H, brd, J=4.5 Hz), 7.370 (2H, d, J=8.0 Hz), 7.814 (2H, d, J=8.0 Hz).

### Example 16

### 20S,3β-(Triisopropylsiloxy)-22,23-bishomopregn-5-ene

A solution of 20R,3β-(triisopropylsiloxy)-22-homopregn-5-en-22-yl p-toluenesulfonate (340 mg, 0.53 mmol) in THF (3 mL) was cooled in an ice bath. Dilithium tetrachlorocuprate (1.16 mL, 0.116 mmol, 0.1 M in THF), was added followed by the dropwise addition of methylmagnesium bromide (0.88 mL), 2.64 mmol, 3.0 M in ether). The mixture was allowed to warm slowly to room temperature and stirred for 22 h. The mixture was cooled in an ice bath and quenched with 0.5 M HCl. The mixture was partitioned between EtOAc and water, extracted with EtOAc (2×), washed with saturated sodium bicarbonate solution, saturated brine, dried over magnesium sulfate, and concentrated to give 253 mg of an off white solid. Recrystallization from isopropanol afforded 20S,3β-(triisopropylsiloxy)-22,23-bishomopregn-5-ene (220 mg, 86%) as a white solid. DE cannot be determined by ¹H NMR. NMR spectrum identical to Example 10.

### Example 17

### 20S,7α/β-Bromo-3β-(triisopropylsiloxy)-22,23-bishomopregn-5-ene

Sodium bicarbonate (1.90 g, 22.6 mmol) and 1N,3N-dibromo-5,5-dimethylhydantoin (0.97 g, 3.39 mmol) were added to a solution of 20S,3β-(triisopropylsiloxy)-22,23-bishomopregn-5-ene (2.20 g, 4.52 mmol) in cyclohexane (80 mL), which was sparged with nitrogen, and then stirred on a 90 oC oil bath for 30 minutes. The reaction mixture was allowed to cool to room temperature, and the solids were removed by vacuum filtration. The solvent was removed under reduced pressure to give crude 20S,7α/β-Bromo-3β-(triisopropylsiloxy)-22,23-bishomopregn-5-ene, as a viscous light yellow oil which was used directly in the next step. ¹H NMR (CDCl₃ 500 MHz) δ: 0.733 (3H, s), 0.856 (3H, t, J=7.2 Hz), 0.946 (3H, d, J=6.6 Hz), 1.052 (3H, s), 1.085 (21H, s), 1.0-1.56 (10H, m), 1.74-2.06 (7H, m), 2.27-2.40 (2H, m), 3.649 (1H, approx septet, J=4.6 Hz), 4.745 (1H, sl brs), 5.725 (1H, sl brd J=5.3 Hz).

### Example 18

### 20S,7β-(4-Chlorophenylthio)-3\tilde{β}(triisopropylsiloxy)-22,23-bishomopren-5-ene

Tetra-n-butylammonium bromide (2.91 g, 9.04 mmol) was added in one portion to a solution of crude 20S,7-α/β-bromo-3β-(triisopropylsiloxy)-22,23-bishomopregn-5-ene (4.52 mmol, obtained from the previous reaction) in toluene/acetone (4:1, 40 mL), stirred on an ice bath under nitrogen. After 2 hours, triethylamine (0.94 mL, 6.78 mmol) and 4-chlorothiophenol (0.65 g, 4.52 mmol) were added sequentially, and the ice bath was removed, and the reaction mixture was stirred at 25° C. for 2 hours. The reaction was worked up by pouring onto water (100 mL), and extracting with EtOAc (2×50 mL). The combined organic extracts were washed with dilute hydrochloric acid (0.5 M, 50 mL), saturated sodium bicarbonate solution (50 mL), saturated brine (50 mL) and dried (MgSO₄). The solvent was removed under reduced pressure to give crude 20S,7β-(4-chlorophenylthio)-3\tilde{β}(triisopropylsiloxy)-22,23-bishomopregn-5-ene (3.17 g, quant) as a light orange gum. ¹H NMR (CDCl₃ 500 MHz) δ: 0.596 (3H, s), 0.721 (3H, s), 0.853 (3H, d, J=6.5 Hz), 0.860 (3H, t, J=6.5 Hz), 1.085 (21H, s), 0.95-1.95 (16H, m), 1.98 (1H, brd), 2.20 (1H, brt), 2.28 (1H, brd), 3.308 (1H, sl brd, J=8.5 Hz), 3.554 (1H, approx septet, J=4.6 Hz), 5.343 (1H, sl brd J=5.3 Hz), 7.277 (2H, d, J=8.6 Hz), 7.306 (2H, d, J=8.6 Hz).

### Example 19

### 20S,7β-(4-Chlorophenylsulfinyl)-3-β-(triisopropylsiloxy)-22,23-bishomopregn-5-ene

m-Chloroperoxybenzoic acid (77%, 1.11 g, 4.95 mmol) was added in one portion to a solution of crude 20S,7β-(4-chlorophenylthio)-3\tilde{β}(triisopropylsiloxy)-22,23-bishomopregn-5-ene (3.17 g, 4.52 mmol) in EtOAc (50 mL) stirred under nitrogen at 0° C. After 1 hour the reaction mixture was diluted with further EtOAc (100 mL) and rinsed with saturated sodium bicarbonate solution (2×100 mL), saturated brine (50 mL) and dried (MgSO₄). The solvent was removed rigorously under reduced pressure without heating to give crude 20S,7β-(4-chlorophenylsulfinyl)-3 β-(triisopropylsiloxy)-22,23-bishomopregn-5-ene (3.14 g, quant) as a light yellow glassy foam. ¹H NMR Major isomer only. (CDCl₃ 500 MHz) δ: 0.052 (3H, s), 0.706 (3H, s), 0.865 (3H, d, J=6.5 Hz), 0.871 (3H, d, J=6.5

Hz), 1.083 (21H, s), 0.95-2.10 (18H, m), 2.43 (1H, brd), 3.551 (1H, approx septet, J=4.6 Hz), 3.664 (1H, sl brd, J=8.7 Hz), 5.773 (1H, sl brs), 7.40-7.50 (4H, m).

### Example 20

### 20S,3β-(Triisopropylsiloxy)-22,23-bishomopregna-5,7-diene

A stirred solution of crude 20S,7β-(4-chlorophenylsulfinyl)-3β-(triisopropylsiloxy)-22,23-bishomopregn-5-ene (3.14 g, 4.52 mmol) and triethylamine (1.38 mL, 9.9 mmol) in toluene (40 mL) was heated to 70° C. under nitrogen for 4 hours. The reaction mixture was allowed to cool, poured onto water (100 mL) and EtOAc (50 mL). The layers were separated, and the aqueous phase was extracted with EtOAc (2×25 mL). The combined organic extracts were washed with dilute hydrochloric acid (0.5 M, 50 mL), saturated sodium bicarbonate solution (50 mL) and saturated brine (50 mL) and dried (MgSO₄). The solvent was removed under rescued pressure to give 3.10 g of light orange gum which was purified by silica gel chromatography (1.5% EtOAc/hexanes, solid loaded in toluene) and recrystallization from isopropanol to give 20S,3β-(triisopropylsiloxy)-22,23-bishomopregna-5,7-diene (1.17 g, 54%) as light yellow crystals. This contained about 5% bis(4-chlorophenyl) disulfide and about 5% of the monoene starting material. ¹H NMR (CDCl₃ 500 MHz) δ: 0.639 (3H, s), 0.865 (3H, t, J=7.2 Hz), 0.967 (3H, s), 1.084 (21H, m), 1.17-1.47 (6H, m), 1.52-1.78 (6H, m), 1.85-1.94 (4H, m), 1.98 (1H, brt), 2.08 (1H, brd), 2.33-2.39 (1H, brt), 2.94 (1H, brd), 3.696 (1H, approx septet, J ˜4.5 Hz), 5.410 (1H, narrow m), 5.578 (1H, dd, J=5.5, 2.3 Hz).

### Example 21

### 7α-Bromo-3,O-(t-butyldimethylsilyl)pregn-5-en-3β-ol-20-one

A suspension of 3,O-(t-butyldimethylsilyl)pregn-5-en-3 β-ol-20-one (430.6 mg, 1.0 mmol), 1,3-dibromo-5,5-dimethylhydantoin (181.0 mg, 0.633 mmol), calcium carbonate (24.4 mg, 0.244 mmol) and 2,2′-azobis(isobutyronitrile) (6.2 mg, 0.0378 mmol) in cyclohexane (10 mL) was degassed by an Ar sparge, and heated to 75° C., with stirring under nitrogen for 30 minutes. The mixture was filtered hot, and the residue was rinsed with hot cyclohexane (5 mL). The solvent was removed under reduced pressure at 45° C., and the residual partially solidified light yellow oil was sonicated, and kept at 4° C. for 68 h. The solids were collected by vacuum filtration and rinsed with cyclohexane (1.0 mL) to give 7α-bromo-3,O-(t-butyldimethylsilyl)pregn-5-en-3β-ol-20-one (303.2 mg, 59.5%) as a pale yellow solid. A further amount (17.7 mg, 2.9%) was recovered from the mother liquors. ¹H NMR (CDCl₃ 500 MHz) δ: 0.085 (6H, s), 0.686 (3H, s), 0.903 (9H, s), 1.041 (3H, s), 1.16-1.26 (2H, m), 1.38-1.9.2 (12H, m), 2.07 (1H, brd), 2.146 (3H, s), 2.15-2.27 (2H, m), 2.638 (1H, t, J=9.3 Hz), 3.584 (1H, approx septet, J=4.6 Hz), 4.744 (1H, narrow m), 5.725 (1H, d, J=3.9 Hz).

### Example 22

### 3,O-(t-butyldimethylsilyl)-7β-(4-chlorophenylthio)pregn-5-en-3β-ol-20-one

7α-Bromo-3,O-(t-butyldimethylsilyl)pregn-5-en-3β-ol-20-one (228 mg, 0.448 mmol) in CH₂Cl₂/MTBE (1:1, 2 mL) was added dropwise over 3 minutes to a thick white slurry of 4-chlorothiophenol (71.8 mg, 0.496 mmol) and DBU (76.8 mg, 0.504 mmol) in MTBE (1.0 mL) stirred under nitrogen at 0° C. After 1 hour, the reaction mixture was quenched with dilute hydrochloric acid (0.5 mL, 10 mL), and the organic phase was extracted with MTBE (2×10 mL). The combined organic extracts were washed with water (10 mL), dilute NaOH solution (0.2 M, 10 mL), water (10 mL) and saturated brine (10 mL) and dried (MgSO4). The solvent was removed rigorously under reduced pressure to give 3,O-(t-butyldimethylsilyl)-7β-(4-chlorophenylthio)pregn-5-en-3β-ol-20-one (238 mg, 92.66%) as a white solid foam. ¹H NMR (CDCl₃ 500 MHz) δ: 0.074, 0.788 (3H, 3H, 2s), 0.576 (3H, s), 0.682 (3H, s), 0.911 (9H, s), 0.91-1.08 (3H, m), 1.27-1.78 (8H, m), 1.82-1.92 (1H, m), 1.96-2.07 (2H, m), 2.158 (3H, s), 2.15-2.28 (3H, m), 2.542 (1H, t, J=9.5 Hz), 3.306 (1H, d, J=8.8 Hz), 3.478 (1H, approx septet, J=5.2 Hz), 5.353 (1H, s), 7.279, 7.323 (2H, 2H, ABq, J=8.5 Hz).

### Example 23

### 3,O-(t-butyldimethylsilyl)-7β-(4-chlorophenylsulfinyl)pregn-5-en-3β-ol-20-one

m-Chloroperoxybenzoic acid (77%, 219.2 mg, 0.978 mmol) was added to a light yellow solution of give 3,O-(t-butyldimethylsilyl)-7β-(4-chlorophenylthio)pregn-5-en-3β-ol-20-one (567.3 mg, 0.989 mmol) in dichloromethane (5.0 mL) stirred under nitrogen at 0 oC. After 30 minutes further mCPBA (77%, 11.5 mg, 0.051 mmol) was added, and after 1 hour the cold solution was quenched by addition of dilute NaOH solution, (0.25 M, 10 mL), and the organic phase was extracted with dichloromethane (2×10 mL). The combined extracts were washed with dilute NaOH solution (0.25 M, 10 mL), water (10 mL), saturated brine (10 mL), and dried (Na2SO4). The solvent was removed rigorously under reduced pressure to give 3,O-(t-butyldimethylsilyl)-7β-(4-chlorophenylsulfinyl)pregn-5-en-3β-ol-20-one (523.2 mg, 89.76%) as a very pale yellow foamed glass.

### Example 24

### 3β-(t-Butyldimethylsiloxy)pregna-5,7-dien-20-one

A pale yellow solution of 3,O-(t-Butyldimethylsilyl)-7β-(4-chlorophenylsulfinyl)pregn-5-en-3β-ol-20-one (518.9 mg, 0.88 mmol) and triethylamine (0.25 mL) in toluene (5 mL) was stirred under nitrogen at 70° C. for 4 hours the mixture darkening somewhat. The yellow solution was filtered through a small pad of silica gel (3 cm×3.4 cm) with gentle suction, and the silica gel was washed with 5%, then 10% ethylacetate/hexanes (100 mL, 100 mL) collecting 50 mL fractions. The appropriate fractions were concentrated under reduced pressure to give 3 β-(t-butyldimethylsiloxy)pregna-5,7-dien-20-one (304.2 mg, 80.6%) as a white solid. ¹H NMR (CDCl₃ 500 MHz) δ: 0.095 (6H, s), 0.602 (3H, s), 0.921 (9H, s), 0.954 (3H, s), 1.306 (1H, d of t, J_{d}=3.5 Hz, Jₜ=13.5 Hz), 1.49-1.63 (3H, m), 1.67-1.92 (6H, m), 2.02-2.09 (2H, m), 2.12-2.29 (2H, m), 2.174 (3H, s), 2.33-2.41 (2H, m), 2.651 (1H, t, J=9.0 Hz), 3.619 (1H, approx septet, J=5 Hz), 5.446 (1H, narrow m), 5.583 (1H, d, J=5.5 Hz).

### Example 25

### 3β-Pregn-5-enol-20-one acetate

4-(N,N-dimethylamino)pyridine (0.12 g, 1.0 mmol) was added to a white slurry of 3β-pregn-5-enol-20-one (9.50 g, 30 mmol) in a mixture of triethylamine (10 mL) and acetic anhydride (6.0 mL) stirred vigourously under nitrogen at 25° C. Within a minute the slurry liquefied slightly, and an exotherm was noted. After 3 min the slurry thickened to a paste, and after about 20 minutes began to yellow. After 30 minutes, the reaction mixture was stirred on an ice-bath and ice-water (150 mL) was added dropwise over 5 minutes. After a further 20 minutes stirring on the ice-bath the reaction mixture was Buchner filtered, and the residue was rinsed with ice-water (4×50 mL). The residue was air dried, and then dried in a vacuum oven at 50° C. for 4 hours to give 3β-pregn-5-enol-20-one acetate (10.65 g, 99%) as a very pale yellow free-flowing solid. ¹H NMR (CDCl₃ 500 MHz) δ: 0.657 (3H, s), 1.046 (3H, s), 0.99-1.07 (1H, m), 1.13-1.31 (3H, m), 1.44-1.77 (8H, m), 1.87-1.94 (2H, m), 1.97-2.12 (2H, m), 2.06 (3H, s), 2.16 (3H, s), 2.16-2.25 (1H, m), 2.19-2.38 (2H, m), 2.56 (1H, t, J=9.0 Hz), 4.63 (1H, approx septet, J=5 Hz), 5.40 (1H, d, J=6.0 Hz).

### Example 26

### 3β,7α-Bromopregn-5-enol-20-one acetate

3β-Pregn-5-enol-20-one acetate (3.585 g, 10.0 mmol), 1,3-dibromo-5,5-dimethylhydantoin (1.876 g, 6.561 mmol), calcium carbonate (201 mg, 2.0 mmol) and 2,2′-azobis(isobutyronitrile) (41 mg, 0.25 mmol) were suspended in cyclohexane (100 mL) and degassed by a vigorous argon sparge, prior to being placed under nitrogen and stirred at 55° C. The reaction mixture took on a pale yellow cast after 5 minutes, and around 20 minutes turned pale orange before decolorizing around 24 minutes. Meanwhile the initial fine suspension became a largely flocculent precipitate around 15 minutes, and tlc (20% EtOAc/hexanes) showed very little starting material. After 27 minutes the mixture was removed from the heat, and stirring was discontinued, giving a very pale yellow solution and a white precipitate. At 30 minutes the reaction mixture was filtered through a medium frit under slight vacuum, and the residue was rinsed with cold cyclohexane (20 mL). The combined organic filtrates were stripped on a rotorvap at 30° C. or below, to a total volume of about 10 mL of a light yellow liquid with a granular precipitate, which was allowed to stand at 25° C. for 22 hours, during which time it became brighter yellow and precipitated further. The solid precipitate was collected by Buchner filtration, rinsed with cyclohexane (2×2 mL), and air dried to give 3β,7α-bromopregn-5-enol-20-one acetate (2.917 g, 66.68%) as a slightly off-white granular solid. The mother liquors (˜6 mL) were allowed to stand at 25° C. for a further 70 hours giving a further desired compound (386 mg, 8.83%) as a light magnolia solid. Nmr analysis of both crops shows purity in the 95-6% range. ¹H NMR (CDCl₃ 500 MHz) δ: 0.695 (3H, s), 1.067 (3H, s), 1.213 (1H, d of q, J_{d}=12.2 Hz, J_{q}=6.2 Hz), 1.313 (1H, d of t, J_{d}=3.8 hz, Jₜ=13.9 Hz), 1.38-1.47 (2H, m), 1.49-1.97 (9H, m), 2.066 (3H, s), 2.164 (3H, s), 2.05-2.25 (2H, m), 2.37-2.45 (2H, m), 2.644 (1H, t, J=9.2 Hz), 4.67-4.77 (2H, m), 5.776 (1H, d, J=5.1 Hz).

### Example 27

### 3β-Pregna-5,7-dienol-20-one acetate

A solution of tetra-n-butylammonium fluoride in THF (1.0 M, 3 mL, 3.0 mmol), which had been predried over activated molecular sieves, was added to a solution of crude 3β,7α-bromopregn-5-enol-20-one acetate (442.5 mg, ˜1.0 mmol) in THF (5 mL), stirred under nitrogen at 0° C. After 1 hour, the reaction mixture was poured onto water (10 mL), and extracted with MTBE (2×10 mL). The combined organic extracts were washed with water (2×10 mL), saturated brine (10 mL) and dried (MgSO₄). The solvent was removed under reduced pressure and the residual light yellow solid (345.4 mg) was purified by flash chromatography on silica gel, eluting with 12% EtOAc/hexanes, to give pregna-5,7-dienol-20-one acetate (162.2 mg, 45.5%) as white plates. ¹H NMR (CDCl₃ 500 MHz) δ: 0.606 (3H, s), 0.972 (3H, s), 1.399 (1H, d of t, J_{d}=4.0 Hz, Jₜ=10 Hz), 1.50-1.65 (2H, m), 1.68-1.88 (5H, m), 1.92-1.98 (2H, m), 2.03-2.30 (5H, m), 2.074 (3H, s), 2.177 (3H, s), 2.390 (1H, brt, J=12.7 Hz), 2.537 (1H, brd, J=14.5 Hz), 2.658 (1H, t, J=9.1 Hz), 4.735 (1H, septet, J=5 Hz), 5.452 (1H, d, J=2.7 Hz), 5.603 (1H, d, J=3.3 Hz).

### Example 28

### 3β-Pregna-5,7-dienol-20-one

A solution of tetra-n-butylammonium fluoride in THF (1.0 M, 20 mL, 20 mmol), which had been predried over activated molecular sieves, was stirred at 0° C., under nitrogen and 3β,7α-bromopregn-5-enol-20-one acetate (2.914 g, 6.663 mmol) was added in one portion. After 3 hours, the ice-bath was removed, and the reaction mixture was stirred at 25° C. for 30 minutes, and then methanol (20 mL) and potassium carbonate (3.454 g, 25 mmol) were added, and the mixture was stirred vigourously at 25° C. Within an hour the mixture had become a thick beige slurry, and after 2.5 hours the reaction mixture was recooled on an ice-bath with stirring, and cold water (125 mL) was added over 5 minutes. After 30 minutes, the reaction mixture was Buchner filtered, and the residue was rinsed with cold water (2×25 mL). The residue was dried in a vacuum oven at 50° C. for 2 hours, and air dried for 48 hours to give 3β-pregna-5,7-dienol-20-one (1.976 g, 94.3%) as a pale yellowish-beige solid. Nmr analysis shows purity in the 95-6% range. ¹H NMR (CDCl₃ 500 MHz) δ: 0.608 (3H, s), 0.966 (3H, s), 1.344 (1H, brt (J=11.5 Hz), 1.47-1.63 (5H, m), 1.67-1.87 (3H, m), 1.90-1.96 (2H, m), 2.02-2.08 (2H, m), 2.13-2.36 (3H, m), 2.177 (3H, s), 2.509 (1H, brd, J=14.5 Hz), 2.654 (1H, t, J=9.2 Hz), 3.669 (H, septet, J=5.5 Hz), 5.453 (1H, narrow m), 5.611 (1H, narrow m).

### Example 29

### 3β-(t-Butyldimethylsiloxy)pregna-5,7-dien-20-one

t-Butyldimethylsilyl chloride (76.4 mg, 0.507 mmol), 4-(N,N-dimethylamino)pyridine (4.5 mg, 0.037 mmol) and pyridine (0.1 mL) were added to a light yellow slurry of 3β-pregna-5,7-dienol-20-one (127 mg, 0.404 mmol) in DMF (0.5 mL) stirred under nitrogen at 25° C. After 20 hours, the reaction mixture was cooled on an ice bath for 15 minutes, and the solids were collected by Buchner filtration, rinsed with cold DMF, (1.0, 0.5 mL) and dried in a vacuum oven at 50 oC, to give 3β-(t-butyldimethylsiloxy)pregna-5,7-dien-20-one (154.4 mg, 89.1%) as a white solid. ¹H NMR (CDCl₃ 500 MHz) δ: 0.095 (6H, s), 0.602 (3H, s), 0.921 (9H, s), 0.954 (3H, s), 1.306 (1H, d of t, J_{d}=3.5 Hz, Jₜ=13.5 Hz), 1.49-1.63 (3H, m), 1.67-1.92 (6H, m), 2.02-2.09 (2H, m), 2.12-2.29 (2H, m), 2.174 (3H, s), 2.33-2.41 (2H, m), 2.651 (1H, t, J=9.0 Hz), 3.619 (1H, approx septet, J=5 Hz), 5.446 (1H, narrow m), 5.583 (1H, d, J=5.5 Hz).

### Example 30

### 3-(t-Butyldimethylsiloxy)-22-homopregna-5,7-diene-20R,22-epoxide

Potassium hexamethyldisilazane (2.49 g, 12.48 mmol) in THF (15 mL) was added to a slurry of trimethylsulfonium iodide (2.55 g, 12.48 mmol) in THF (15 mL) stirred under nitrogen at 25° C. The slurry was stirred 10 minutes, then toluene (10 mL) was added and the mixture was cooled to −70° C. in a dry ice/isopropanol bath for 15 min. A solution of 3β-(t-butyldimethylsiloxy)pregna-5,7-dien-20-one in toluene (30 mL) was added dropwise over 20 min. The mixture was stirred 1 h at −70° C., then allowed to warm slowly to 0° C. over 2 h. The bath was removed and the mixture was allowed to warm to room temperature and stirred 30 min. The mixture was cooled in an ice bath and quenched by the rapid addition of acetic acid (1 mL). Water (30 mL) was added along with NaHSO₃ (100 mg) and the mixture was allowed to warm to room temperature and stirred for 15 min. The mixture was transferred to a separatory funnel and the layers were separated. The aqueous layer was extracted with MTBE (2×25 mL). The combined organic extracts were washed with saturated sodium bicarbonate solution, brine, dried over magnesium sulfate, and concentrated to give 3β-(t-butyldimethylsiloxy)-22-homopregn-5,7-diene-20R,22-epoxide (2.67 g, 99%) as white glistening plates, which was a greater than 40:1 mixture of diastereoisomers (est. by ¹H NMR). ¹H NMR (CDCl₃ 500 MHz) δ: 0.095 (6H, s), 0.773 (3H, s), ), 0.918 (9H, s), 0.970 (3H, s), 1.25-1.40 (2H, m), 1.403 (3H, s), 1.43-1.67 (5H, m), 1.72-2.03 (8H, m) 2.14 (1H, brd), 2.35-2.39 (2H, m), 2.555 (1H, d, J=4.8 Hz), 3.618 (1H, approx septet, J ˜4.5 Hz), 5.410 (1H, narrow m), 5.576 (1H, d, J=5.4 Hz).

### Example 31

### 20R,3β-(t-Butyldimethylsiloxy)-22-homopregna-5,7-dien-22-al

Magnesium bromide bis-diethyl etherate (40.0 mg, 0.154 mmol) was added in one portion to a solution of 3β-(t-butyldimethylsiloxy)-22-homopregna-5,7-dien-20R,22-epoxide (143.5 mg, 0.308 mmol) in toluene (3.0 mL), stirred under nitrogen at −10° C. After 2 hours, the reaction mixture was stirred at 0° C. for 3 hours, and then quenched by addition of dilute hydrochloric acid (0.1 M, 5 mL). The mixture was extracted with MTBE (10 mL), and the organic phase was washed with water (2×10 mL), saturated brine (10 mL), and dried (MgSO₄). The solvent was removed under reduced pressure to give 20R,3β-(t-butyldimethylsiloxy)-22-homopregn-5-en-22-al (136.4 mg, 95% yield) as a yellow waxy solid. ¹H NMR (CDCl₃ 500 MHz) δ: 0.082 (6H, s), 0.637 (3H, s), 0.909 (9H, s), 0.9321 (3H, s), 1.073 (3H, d, J=6.8 Hz), 1.13-1.32 (2H, m), 1.39-2.02 (14H, m), 2.31-2.37 (2H, m), 3.600 (1H, approx septet, J ˜4.5 Hz), 5.415 (1H, narrow m), 5.561 (1H, d, J=5.3 Hz) 9.589 (1H, d, J=4.1 Hz).

### Example 32

### 20S,3β-(t-Butyldimethylsiloxy)-22,23-bishomopregna-5,7,22-triene

Methyltriphenylphosphonium iodide (405.6 mg, 1.0 mmol) and potassium hexamethyldisilazane (190.1 mg. 0.95 mmol) were stirred together in THF (2.0 mL) under nitrogen at 25° C., and then the bright yellow slurry was cooled to 0° C., and 20R,3β-(t-butyldimethylsiloxy)-22-homopregn-5-en-22-al (133.0 mg, 0.30 mmol) in THF (3.0 mL) was added dropwise over 2 minutes. After 15 minutes the reaction mixture was diluted with hexanes (50 mL), and stirred with celite (1.0 g) at 0° C. for 20 minutes. The reaction mixture was eluted through a small silica gel plug, and the solvent was removed under reduced pressure to give 20S,3 β-(t-butyldimethylsiloxy)-22,23-bishomopregna-5,7,22-triene (103.5 mg, 78%) as a pale yellow waxy solid. ¹H NMR (CDCl₃ 400 MHz) δ: 0.049 (6H, s), 0.587 (3H, s), 0.878 (9H, s), 0.902 (3H, s), 0.929 (3H, d, J=6.6 Hz), 1.02-1.11 (1H, m), 1.20-1.98 (15H, m), 2.02-2.14 (2H, m), 2.28-2.33 (2H, m), 3.57 (1H, approx septet, J ˜4.5 Hz), 4.836 (1H, dd, J=10.1, 2.1 Hz), 4.963 (1H, dd, J=17.1, 2.1 Hz), 5.363 (1H, dt, J_{d}=5.3 Hz, Jₜ=2.8 Hz), 5.534 (1H, d, J=5.3 Hz) 5.717 (1H, ddd, J=9.5, 10.1, 17.1 Hz).

### Example 33

### One flask, 2 Step, Preparation of 20R,3 β-(t-butyldimethylsiloxy)-22-homopregna-5,7-dien-22-ol from 3β-(t-butyldimethylsiloxy)-22-homopregna-5,7-diene-20R,22-epoxide

A suspension of magnesium bromide bis diethyletherate (106 mg, 0.41 mmol) in toluene (4 mL) was cooled in an ice bath. 3β-(t-Butyldimethylsiloxy)-22-homopregna-5,7-dien-20R,22-epoxide (365 mg, 0.82 mmol) was added in one portion. The mixture was stirred for 2 h at 0° C., then MeOH (1 mL) was added followed by sodium borohydride (16 mg, 0.41 mmol). After 20 min., the reaction was quenched by the dropwise addition of 0.5 M HCl. After 10 min, the ice bath was removed and the mixture was transferred to a separatory funnel and extracted with MTBE (2×). The combined organic extracts were washed with saturated sodium bicarbonate solution, saturated brine, dried over magnesium sulfate, and concentrated to give 390 mg of a white solid, which was taken up in toluene (3 mL) with sonication. Flash chromatography (15% EtOAc/hexane) gave 20R,3β-(t-butyldimethylsiloxy)-22-homopregna-5,7-dien-22-ol (267 mg, 73%) as a white solid. ¹H NMR analysis was consistent with a single (R)-alcohol diastereomer of approximately 97% purity (approx. 3% of allylic alcohol byproduct). ¹H NMR (CDCl₃ 500 MHz) δ: 0.093 (6H, s), 0.665 (3H, s), 0.927 (9H, s), 0.961 (3H, s), 1.003 (3H, d, J=6.7 Hz), 1.232 (1H, brs), 1.293 (1H, d of t, J_{d}=3.9 Hz, Jₜ=13.5 Hz), 1.35-1.48 (2H, m), 1.51-1.83 (8H, m), 1.85-2.03 (5H, m), 2.33-2.37 (2H, m), 3.554 (1H, dd, J=10.3, 11.5 Hz), 3.614 (1H, approx septet, J ˜4.5 Hz), 3.771 (1H, sl brd, J=9.1 Hz), 5.418 (1H, narrow m), 5.578 (1H, d, J=5.3 Hz).

### Example 34

### 20R,3 β-(t-Butyldimethylsiloxy)-22-homopregna-5,7-dien-22-yl p-toluenesulfonate

To a solution of 20R,3β-(t-butyldimethylsiloxy)-22-homopregn-5,7-dien-22-ol (261 mg, 0.59 mmol) in dichloromethane (5 mL) was added triethylamine (0.25 mL, 1.76 mmol) and a crystal of 4-(N,N-dimethylamino)pyridine. p-Toluenesulfonyl chloride (134 mg, 0.70 mmol) was added and the solution was stirred for 18 h. The solution was partitioned between EtOAc and water and extracted with EtOAc (2×). The combined organic extracts were washed with saturated sodium bicarbonate solution, saturated brine, dried over magnesium sulfate, and concentrated to give 345 mg of a light yellow gum. Flash chromatography (15% EtOAc/hexane) gave 20R,3 β-(t-butyldimethylsiloxy)-22-homopregna-5,7-dien-22-yl p-toluenesulfonate (236 mg, 67%) as a white foam. ¹H NMR (CDCl₃ 500 MHz) δ: 0.094 (6H, s), 0.562 (3H, s), ), 0.920 (3H, d, J=6.3 Hz), 0.927 (9H, s), 0.935 (3H, s), 1.204 (1H, d of t, J_{d}=4.5 Hz, Jₜ=13.0 Hz), 1.23-1.45 (3H, m), 1.43-1.98 (12H, m), 2.33-2.37 (2H, m), 2.480, (3H, s), 3.610 (1H, approx septet, J ˜4.5 Hz), 3.888 (1H, dd, J=7.1, 9.3 Hz), 4.158 (1H, dd, J=3.5, 9.3 Hz), 5.390 (1H, narrow m), 5.561 (1H, d, J=5.4 Hz) 7.373 (2H, d, J=8.0 Hz), 7.818 (2H, d, J=8.) Hz).

### Example 35

### 20S,3 β-(t-Butyldimethylsiloxy)-22,23-bishomopregna-5,7-diene

A solution of 20R,3β-(t-butyldimethylsiloxy)-22-homopregna-5,7-dien-22-yl p-toluenesulfonate (230 mg, 0.38 mmol) in THF (3 mL) was cooled in an ice bath. Dilithium tetrachlorocuprate (0.1 M in THF, 0.84 mL, 0.084 mmol) was added followed by the dropwise addition of methylmagnesium bromide (3.0 M in ether, 0.64 mL, 1.92 mmol). The mixture was allowed to warm slowly to room temperature and stirred for 23 h. The mixture was cooled in an ice bath and quenched with 0.5 M HCl. The mixture was partitioned between EtOAc and water and extracted with EtOAc (2×). The combined organic extracts were washed with saturated sodium bicarbonate solution, saturated brine, dried over magnesium sulfate, and concentrated to give 20S,3β-(t-butyldimethylsiloxy)-22,23-bishomopregna-5,7-diene (169 mg, 99%) of the title compound as an off-white solid. ¹H NMR (CDCl₃ 500 MHz) δ: 0.093 (6H, s), 0.638 (3H, s), ), 0.859 (3H, d, J=6.7 Hz), 0.866 (3H, t, J=7.1 Hz), 0.927 (9H, s), 0.960 (3H, s), 1.17-1.47 (6H, m), 1.51-1.98 (1H, m), 2.07 (1H, brd), 2.33-2.39 (2H, m), 3.617 (1H, approx septet, J ˜4.5 Hz), 5.408 (1H, narrow m), 5.577 (1H, d, J=5.4 Hz).

### Example 36

### 20R,3β-(t-Butyldimethylsiloxy)-22-homopregna-5,7-dien-22-yl bromide

Triphenylphosphine (65.1, 65.4, 64.9 mg) was added in three batches at intervals of 10 minutes to a colourless solution of 20R,3β-(t-butyldimethylsiloxy)-22-homopregna-5,7-dien-22-ol (222.3 mg, 0.50 mmol), carbon tetrabromide (247.1 mg, 0.745 mmol) and collidine (103.2 mg, 0.852 mmol) in dichloromethane (5 mL), stirred under nitrogen at 25° C. The reaction mixture became a pale yellow solution, and 45 minutes after the first phosphine addition, celite (2 g) was added followed by hexanes (20 mL). The mixture was passed through a silica gel plug (3×3.4 cm), eluting with 5% EtOAc/hexanes (200 mL), collecting four fractions. The second fraction was concentrated under reduced pressure, and the volatiles removed on a vacuum pump to give 20R,3β-(t-butyldimethylsiloxy)-22-homopregna-5,7-dien-22-yl bromide (223.1 mg, 87.9%) as a white crystalline solid. ¹H NMR (CDCl₃ 500 MHz) δ: 0.064 (6H, s), 0.658 (3H, s), ), 0.915 (9H, s), 0.956 (3H, s), 1.055 (3H, d, J=6.5 Hz), 1.230 (1H, dt, J_{d}=4.1 Hz, Jₜ=13.8 Hz), 1.37-2.04 (XXH, m), 2.33-2.38 (2H, m), 3.346 (1H, dd, J=9.8, 6.3 Hz), 3.624 (1H, approx septet, J ˜4.5 Hz), 3.661 (1H, dd, J=9.9, 3.1 Hz), 5.413 (1H, narrow m), 5.571 (1H, d, J=4.5 Hz).

### Example 37

### Photolysis of 20S,3β-(t-Butyldimethylsiloxy)-22,23-bishomopregna-5,7-diene to 20S,6Z,3β-(t-Butyldimethylsiloxy)-22,23-bishomo-9,10-secopregna-5(10),6,8(9)-triene

A 500 mL “Ace Glass” photo-reaction vessel with a quartz immersion well, magnetic stirrer, thermocouple, nitrogen inlet tube, drying tube and cooling bath, was charged with a solution of 20S,3β-(t-butyldimethylsiloxy)-22,23-bishomopregna-5,7-diene (5.00 g, 11.3 mmol) and ethyl 4-dimethylaminobenzoate (0.116 g, 0.60 mmol) in MTBE (500 mL). The solution was thoroughly degassed with a gentle nitrogen sparge overnight, and cooled to between −10 and −20° C., and was then photolysed with a Hanovia medium pressure mercury lamp for 3 h. At this point nmr analysis shows about a 6:47:47 mixture of starting material (Compound 39), pre-Vitamin D isomer (compound 54), and Tachy-isomer (compound 55). NMR. Olefinic protons, ppm: starting diene: 5.55 (m), 5.38 (m); Pre-isomer: 5.94 (d, 12.3 Hz), 5.66 (d, 12.3 Hz) 5.49 (m); Tachy-isomer: 6.70 (d, 16.2 Hz), 6.00 (d, 16.2 Hz). 9-acetylanthracene (0.026 g, 0.118 mmol) was added to the solution and a uranium glass filter was placed in the lamp well, to cut off shorter wavelengths than 350 nm, and irradiation was continued at −10° C. for another 20 minutes, when nmr analysis showed almost complete disappearance of tachy isomer (55). The solution was then stripped to dryness to give crude 20S,6Z,3 β-(t-butyldimethylsiloxy)-22,23-bishomo-9,10-secopregna-5(10),6,8(9)-triene as a light yellow oil.

### Example 38

### Thermal rearrangement of 20S,6Z,3β-(t-butyldimethylsiloxy)-22,23-bishomo-9,10-secopregna-5(10),6,8(9)-triene to 20S,7Z7E,3β-(t-butyldimethylsiloxy)-22,23-bishomo-9,10-secopregna-5,7,10(19)-triene

Crude 20S,6Z,3β-(t-butyldimethylsiloxy)-22,23-bishomo-9,10-secopregna-5(10),6,8(9)-triene (˜5 g, ˜11.3 mmol) from the previous photolysis was dissolved in warm EtOH (120 mL), and refluxed under nitrogen in the dark for 12 hours. The reaction mixture was allowed to cool slowly to 25° C., and was stirred at that temperature for a further 72 hours, at which time the ratio of product to starting material (by mnr) had improved from 1:2.9 at the end of the reflux to 1:5.2. (Pre-isomer: 5.94 (d, 12.3 Hz), 5.66 (d, 12.3 Hz) 5.49 (m); Vitamin D type-isomer: 6.16 (d, 11.4 Hz), 6.00 (d, 11.4 Hz), 5.00 (m˜bs), 4.77 (m˜bs). The crude ethanolic solution was used directly in the next step.

### Example 39

### (1R,2S,6R,7R)-6-Methyl-7-([1S]methylprop-1-yl)bicycle[4.3.0]nonan-2-ol

A stirred solution of crude 20S,7Z,7E,3β-(t-butyldimethylsiloxy)-22,23-bishomo-9,10-secopregna-5,7,10(19)-triene (˜5 g, ˜11.3 mmol) in EtOH (120 mL) with a fritted gas inlet was cooled to −70° C. under nitrogen. Once the reaction vessel temperature had been stabilized, an oxygen sparge was initiated, and after 10 minutes, the ozonizer was turned on, and ozone was passed through the solution, producing a noticeable exotherm, but cooling was adjusted to keep the temperature at or below −65° C. The solution became greenish as the reaction ceased to be exothermic, and after a few minutes further, ozone addition was stopped, and the reaction vessel was purged with nitrogen for 10 minutes. Then NaBH₄ (4.5 g, 119 mmol) was added in portions at −70° C. with stirring, and the reaction mixture was allowed to warm up slowly to 25° C. over several hours. After 12 hours, the volatiles were removed under reduced pressure, and the residue was added to water (60 mL), and extracted with MTBE (2×60 mL). The combined organic extracts were washed with saturated brine (40 mL), dried, (MgSO₄), and the solvent was removed under reduced pressure to give a thick oily residue which was purified by silica gel column chromatography eluting with 2-5% EtOAc/heptanes, to give (1R,2S,6R,7R)-6-methyl-7-([1S]methylprop-1-yl)bicycle[4.3.0]nonan-2-ol (0.86 g, 36.18%) as a very pale yellow oil.

### Example 40

### (1R,6R,7R)-6-Methyl-7-([1S]methylprop-1-yl)bicycle[4.3.0]nonan-2-one

Pyridinium dichromate (2.26 g, 6.00 mmol) was added to a solution of (1R,2S,6R,7R)-6-methyl-7-([1S]methylprop-1-yl)bicycle[4.3.0]nonan-2-ol (0.84 g, 3.99 mmol) in dichloromethane (20 mL), stirred under nitrogen at 25° C. for 7 hours. The reaction mixture was then passed through a short silica gel column, eluting with MTBE. The solvent was removed under reduced pressure at 25° C. to give (1R,6R,7R)-6-methyl-7-([1S]methylprop-1-yl)bicycle[4.3.0]nonan-2-one (0.82 g, 98.6%) as a pale yellow liquid.

### Example 41

### (20S)-1 α-(t-Butyldimethylsiloxy)-3O-(t-butyldimethylsilyl)-2-methylene-19-nor-22,23-bishomopregnacalciferol

n-Butyl lithium (1.6 M in hexanes, 3.3 mL, 5.28 mmol) was added dropwise over 5 min to a solution of P-(2-{[3S,5R]-3,5-bis(t-butyldimethylsiloxy)-4-methylidenecyclohexylidene}ethyl)diphenylphosphine oxide (3.45 g, 5.92 mmol) in THF (30 mL). stirred under nitrogen at −70° C. After 15 minutes a solution of (1R,6R,7R)-6-methyl-7-([1S]methylprop-1-yl)bicycle[4.3.0]nonan-2-one (0.82 g, 3.94 mmol) in THF (5 mL), was added over 5 minutes to the deep red solution. After 3 hours, the reaction mixture was allowed to warm up slowly to 25° C., and the tan slurry was stirred at that temperature for a further 10 hours. The reaction mixture was cooled on an ice bath and water (0.80 mL) was added dropwise. The volatiles were removed under reduced pressure, and the residue was diluted with water (35 mL), and extracted with heptanes (35, 15 mL). The combined extracts were washed with saturated brine (15 mL), dried (MgSO₄) and concentrated to 5 mL under reduced pressure. The solution was purified by silica gel chromatography eluting with heptanes, and the solvent was removed under reduced pressure to give (20S)-1α-(t-butyldimethylsiloxy)-3O-(t-butyldimethylsilyl)-2-methylene-19-nor-22,23-bishomopregnacalciferol (1.80 g, 79.7%) as a pale yellow oil.

### Example 42

### (20S)-1-Hydroxy-2-methylene-19-nor-22,23-bishomopregnacalciferol

Tetra-n-butylammonium fluoride hydrate (7.80 g, 30 mmol) was added to a solution of (20S)-1α-(t-butyldimethylsiloxy)-3O-(t-butyldimethylsilyl)-2-methylene-19-nor-22,23-bishomopregnacalciferol (1.72 g, 3.00 mmol) in THF (25 mL), stirred under nitrogen at 20° C. A modest endotherm was noted, and the pale gray solution was stirred at 20° C. for a further 20 hours. The solution was concentrated under reduced pressure with slight heating, and the residual solution was added to water (50 mL) and was extracted with MTBE (50 mL). The organic phase was washed with water (3×15 mL), saturated brine (15 mL) and dried rapidly over MgSO₄. The solvent was removed under reduced pressure, and the residue was triturated with acetonitrile (10 mL), and kept overnight at 25° C. The solids were collected by Buchner filtration, rinsed with cold acetonitrile (2×2 mL), and dried in vacuo to give (20S)-1α-Hydroxy-2-methylene-19-nor-22,23-bishomopregnacalciferol (0.88 g, 85.1%) as a white crystalline solid.

The steroids and Vitamin D derivatives disclosed herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound which may be made via this process and a pharmaceutically acceptable carrier. One or more compounds which may be made via this process may be present in association with one or more non-toxic pharmaceutically acceptable carriers and/or diluents and/or adjuvants, and if desired other active ingredients. The pharmaceutical compositions containing compounds which may be made via this process may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds which may be made via this process may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds which may be made via this process disclosed herein may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.0001 to 0.25% w/w, preferably, 0.0005-0.1% w/w and most preferably 0.0025-0.05% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.000001 mg to about 0.01 mg per kilogram of body weight per day are useful in the treatment of the above-indicated conditions (about 0.5 μg to about 0.5 mg per patient per day). The amount of active ingredient that may be combined with the carrier materials to produce a single dosage form will vary depending upon the host treated and the particular mode of administration. Dosage unit forms will generally contain between from about 1 μg to about 5 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A method of preparing the compound of the formula: 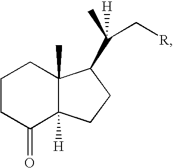
where R is alkyl, alkenyl, alkynyl, —O-alkanoyl, alkoxy, alkoxyalkoxy, —O-silyl, OH, cycloalkyl, aryl, heteroaryl, or heterocycloalkyl, wherein each is optionally substituted with one or more groups that are independently alkyl, halogen, alkoxy, amino, monoalkylamino, dialkylamino, cyano, —O-trityl, or —O-pivaloyl,
the method comprising
a) reacting the 3-hydroxy group of pregn-5-en-3β-ol-20-one with a protecting group to form a compound of the formula: 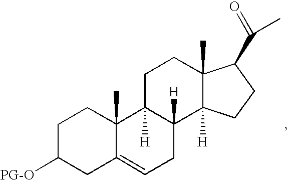
where PG is a protecting group;
b) converting the product from step a) into a compound of the formula: 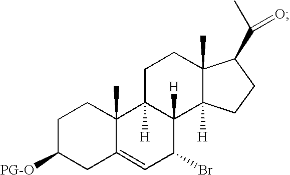
c) converting the product from step b) into a compound of the formula: 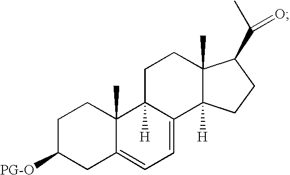
d) if necessary for removal or exchange of the protecting group, converting the product from step c) into a compound of the formula: 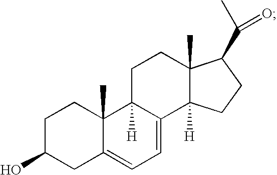
e) if necessary for exchange of the protecting group converting the product from step
d) into a compound of the formula: 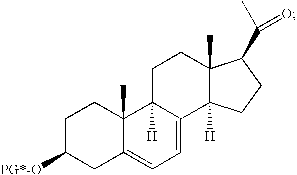
f) converting the product from step e) into a compound of the formula, where PG and PG* may be the same or different: 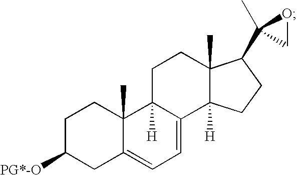
g) converting the product from step f) into a compound of the formula: 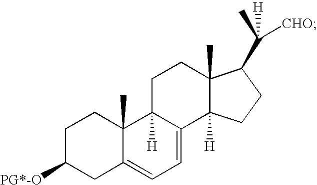
h) converting the product from step g) into a compound of the formula: 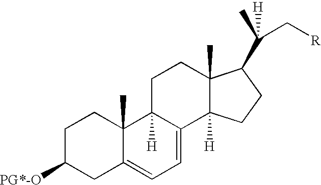
where R represents a desired Vitamin D side chain, which may be a carbon radical singly, doubly or triply bonded to C22, or a carbon radical substituted heteroatom;
i) converting the product from step g) into a compound of the formula: 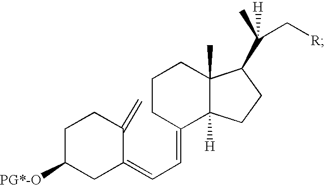
and,
converting the product from step h) into the desired product.

2. The method of claim 1 where R is methyl.

3. The method of claim 1, where PG is an alkanoyl group and PG* is a silyl protecting group.

4. The method of claim 1, where PG is acetate and PG* is the t-butyldimethylsilyl group.

5. The method of claim 1, where PG is acetate and PG* is the t-butyldimethylsilyl group and R is methyl.

6. The method according to claim 1, where the product of step e) is converted to the product of step f) by treatment with CH₂═S(CH₃)₂, in a solvent, at low temperature.

7. The method of claim 6, wherein the solvent is THF with toluene as cosolvent, if required, and PG* is a TBDMS or TIPS group.

8. The method according to claim 1, wherein PG is acetate and PG* is TIPS.

9. The method according to claim 1, wherein the silyl group is TMS, TBDMS, TPS, TIPS, or TBDPS.

10. Intermediates of the formulas:

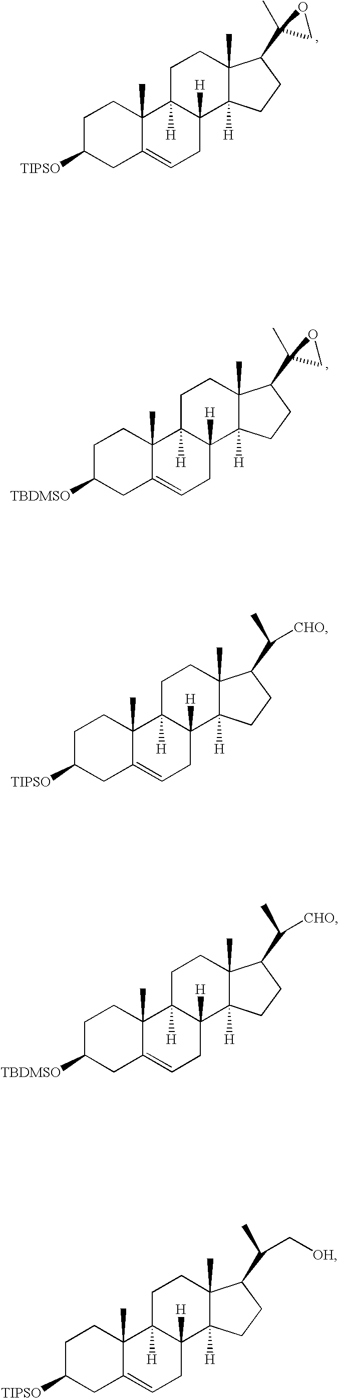

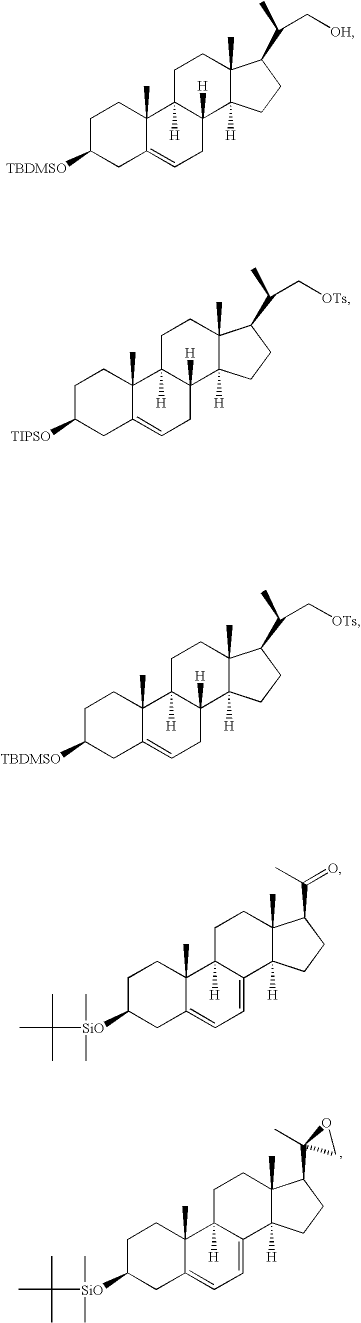

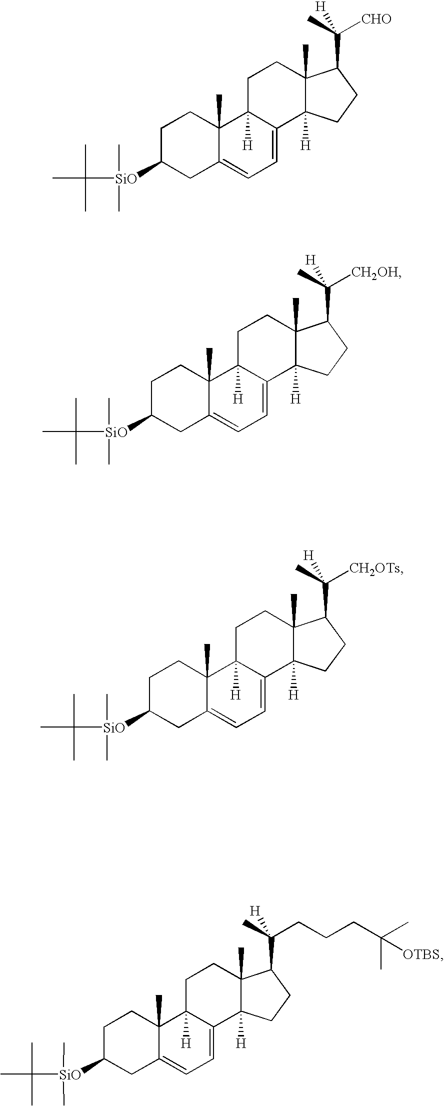

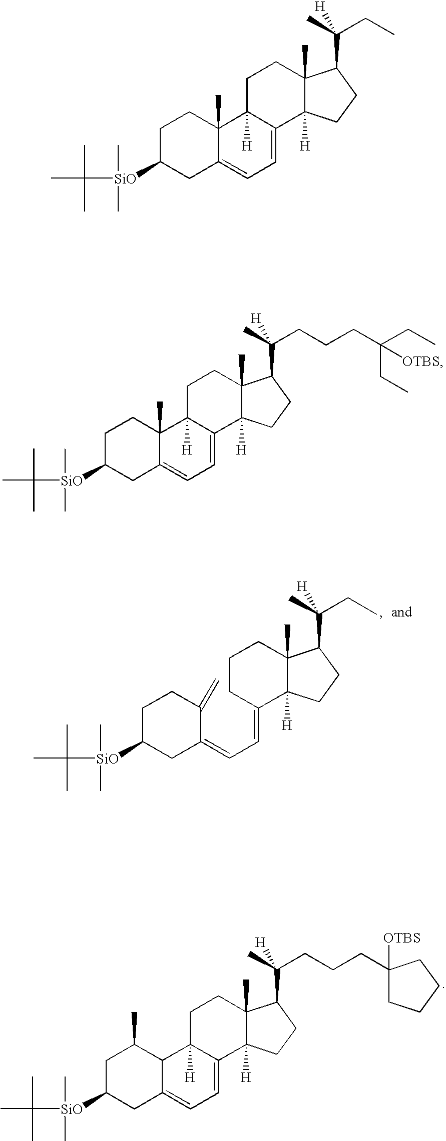

11. Compounds of the formulas:

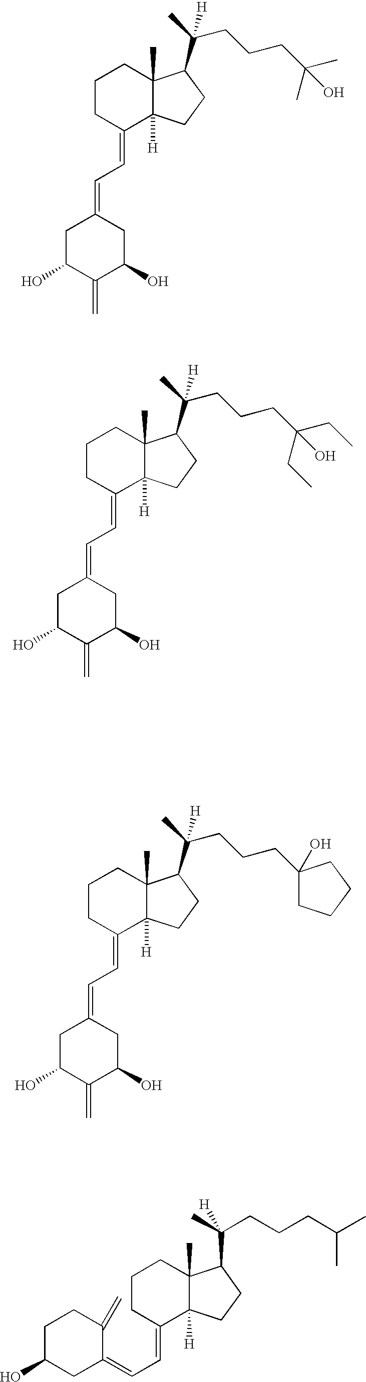

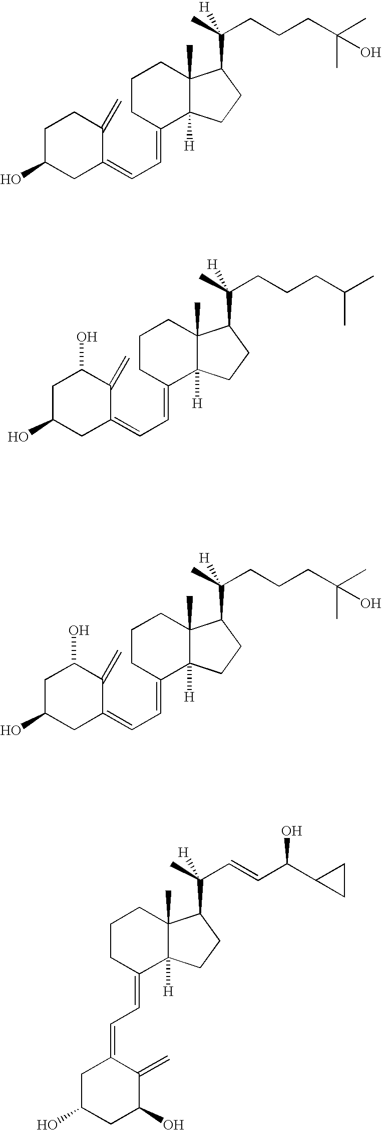

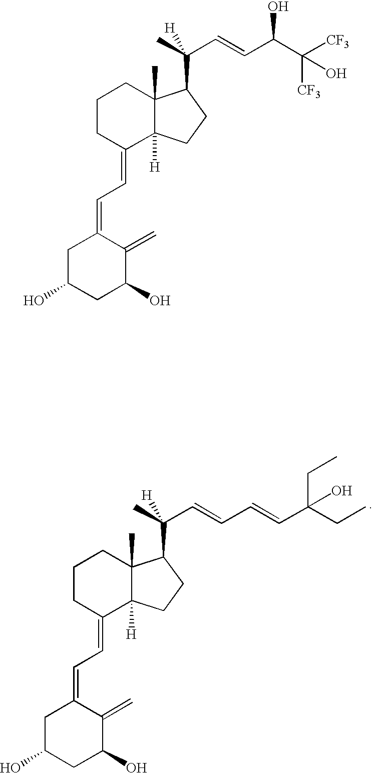

12. The use of one or more of the compounds of claim 10 in the preparation of the compounds of claim 11.

13. A method of producing a compound of the formula: 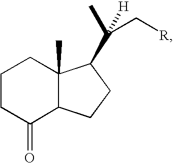
where R is alkyl, alkenyl, alkynyl, —O-alkanoyl, alkoxy, alkoxyalkoxy, —O-silyl OH, cycloalkyl, aryl, heteroaryl, or heterocycloalkyl, wherein each is optionally substituted with one or more groups that are independently alkyl, halogen, alkoxy, amino, monoalkylamino, dialkylamino, cyano, —O-trityl, or —O-pivaloyl,
the method comprising
a) reacting the 3-hydroxy group of pregnenolone with a protecting group to form a compound of the formula: 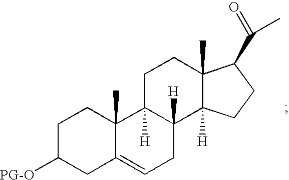
b) converting the product from step a) into a compound of the formula: 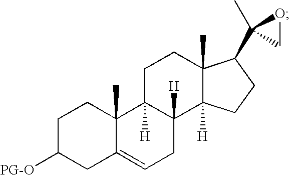
c) converting the product from step b) into a compound of the formula: 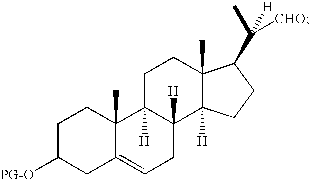
d) converting the product from step c) into a compound of the formula: 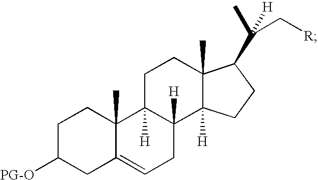
e) converting the product from step d) into a compound of the formula: 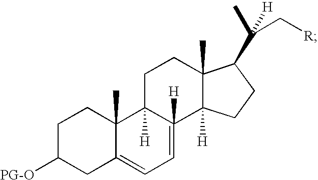
f) converting the product from step e) into a compound of the formula: 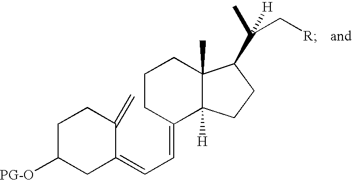
g) converting the product from step f) into the desired product.

14. The method of claim 13, where R is methyl.

15. The method of claim 13, where PG is a C₁-C₄ alkyl, benzyl or silyl group.

16. The method of claim 15, where PG is a silyl group that is TBS, TES, or TIPS.

17. The method according to claim 13, where the product of step e) is converted to the product of step f) by treatment with CH₂═S(CH₃)₂, in a solvent, at low temperature.

18. The method of claim 17, wherein the solvent is THF with toluene as cosolvent, if required, and PG* is a TBDMS or TIPS group.

19. The method according to claim 13, wherein the silyl group is TMS, TBDMS, TPS, TIPS, or TBDPS.

20. A pharmaceutical composition comprising steroids and Vitamin D derivates made using the method of claim 1 or 13 and at least one pharmaceutically acceptable carrier, excipient, adjuvant or glidant.

21. A pharmaceutical composition comprising the compounds of claim 11 and at least one pharmaceutically acceptable carrier, excipient, adjuvant or glidant.

22. The use of the methods of claim 1 or claim 13 to prepare stereospecifically at C20 compounds of the formula 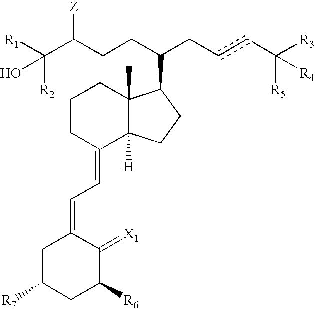
wherein:
the C23-C24 bond may be a single, double or triple bond;
R₁, R₂, R₃ and R₄ are each independently C₁-C₄ alkyl, C₁-C₄ deuteroalkyl, hydroxyalkyl or haloalkyl;
R₅, 6 and R₇ are each independently OH, OC(O)C₁-C₄ alkyl, OC(O)hydroxyalkyl or OC(O)haloalkyl;
X₁ is CH₂;
Z is H, OH, ═O, SH or NH₂.

23. Compounds according to claim 11 of the formulas:

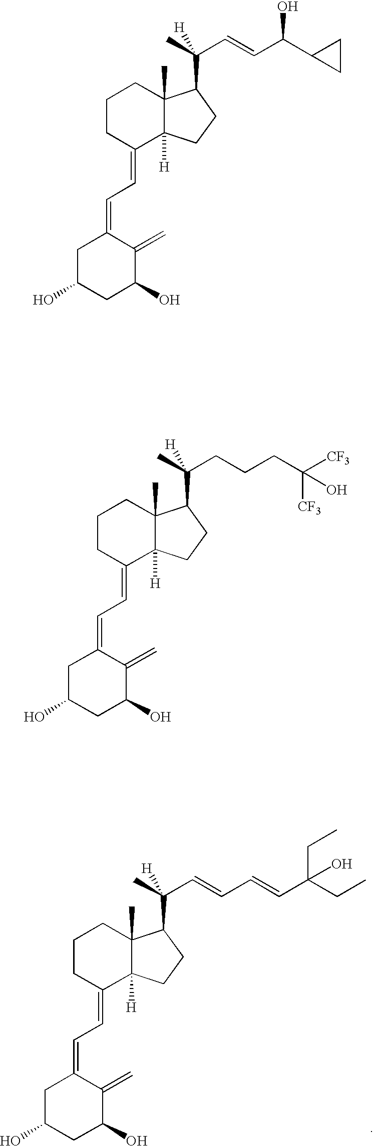

24. The use of the methods of claim 1 or claim 13 to prepare stereospecifically at C20 the compounds of claim 23.
